(19) United States
(12) Patent Application Publication
(10) Pub. No.: US 2026/0000300 A1
(43) Pub. Date: Jan. 1, 2026

(54) INTRAORAL SCAN-BASED GINGIVAL RECESSION MEASUREMENT AND CATEGORIZATION AND ASSESSMENT OF TEMPOROMANDIBULAR DISORDER

(71) Applicant: Align Technology, Inc., San Jose, CA (US)

(72) Inventors: Christopher E. Cramer, Durham, NC (US); Michael Austin Brown, Orlando, FL (US); Michael Chang, Zürich (CH); Matthew David Smith, Raleigh, NC (US)

(21) Appl. No.: 19/320,955

(22) Filed: Sep. 5, 2025

Related U.S. Application Data
(63) Continuation of application No. 19/239,199, filed on Jun. 16, 2025.
(60) Provisional application No. 63/660,913, filed on Jun. 17, 2024.
(60) Provisional application No. 63/661,368, filed on Jun. 18, 2024.

(51) Int. Cl. A61B 5/00; A61B 6/03; A61B 6/51; G06T 7/00; G06T 7/11; G06T 17/00

(52) U.S. Cl. CPC A61B 5/0088; A61B 5/7264; G06T 7/0012; G06T 7/11; G06T 17/00; A61B 6/032; A61B 6/512; G06T 2207/20081; G06T 2207/30036; G06T 2210/41

(57) ABSTRACT

A method is provided for assessing and categorizing temporomandibular disorder (TMD). In some cases, the method can include receiving data representing a potential for TMD of a patient. The method can include processing the data to identify an indicator of the TMD. The method can include identifying a treatment recommendation based on the indicator of the TMD. The method can include providing the treatment recommendation for display on a user device.

### RELATED APPLICATIONS

This application is the continuation of Non-Provisional application Ser. No. 19/239,199, filed Jun. 16, 2025, which claims the benefit of U.S. Provisional Patent Application No. 63/660,913, filed on Jun. 17, 2024, and U.S. Provisional Patent Application No. 63/661,368, filed on Jun. 18, 2024, which are herein incorporated by reference in their entirety.

### TECHNICAL FIELD

The instant specification generally relates to systems and methods for intraoral scan-based gingival recession measurement and categorization, and for assessing Temporomandibular Disorder (TMD).

### BACKGROUND

Gingival recession is condition characterized by the withdrawal of gum tissue from around the teeth, leading to the exposure of tooth roots. This condition, and root exposure, can be problematic in a number of ways. Exposed roots of the teeth lack the protective enamel found on crowns, making them more susceptible to decay and erosion. Gingival recession can also increase the vulnerability of the teeth to other issues such as over-sensitivity around the exposed area, as well as the potential for tooth loss.

Gingival recession is frequently observed as a result of periodontal disease, which causes the supporting gum tissue to deteriorate and withdraw. However, the condition can arise through other factors, such as aggressive brushing, usage of hard-bristled toothbrushes, excessive force, etc. Gingival recession and associated factors affect the structural integrity of the teeth, aesthetics of a smile, and dental sensitivity, thus negatively impacting the quality of life of the affected individuals.

Temporomandibular Disorder (TMD) is a collective term used to describe a group of conditions affecting the temporomandibular joint (TMJ), the masticatory muscles, and the associated structures. The TMJ, located in front of each ear, connects the lower jaw (mandible) to the temporal bone of the skull. This joint plays a crucial role in various functions such as chewing, speaking, and swallowing. Dysfunction in the TMJ or associated muscles can lead to significant discomfort and impairment in these everyday activities.

The symptoms of TMD can vary widely among individuals, but common manifestations include jaw pain or tenderness; headaches; deficiency in maximum opening, lateral and protrusive movements; difficulty in chewing; pain in or around the ear; and clicking, popping, snapping, crepitus, or grating sounds during opening/lateral/protrusive movements of the jaw. In some cases, individuals may experience locking of the jaw joint, making it difficult to open or close the mouth. Additionally, the size, shape and/or appearance of the joint bones may be abnormal (e.g., condylar head, fossa/articular eminence), or the relation/position of the condyle to the articular fossa may be incorrect. The exact etiology of TMD is often multifactorial, involving a combination of genetic, hormonal, environmental, and behavioral factors.

The assessment of TMD typically involves a comprehensive clinical evaluation, which includes a detailed patient history and a physical examination of the jaw and TMJ. Assessment of TMD remains challenging due to the complexity of the disorder and the variability of symptoms. Accurate diagnosis is critical for the effective management of TMD, which may include a combination of treatments such as orthodontia, physical therapy, medication, occlusal splints, and, in severe cases, surgical intervention. Improved methods for assessing TMD are essential to enhance diagnostic accuracy and optimize patient outcomes.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In a first implementation, a system comprises a memory and a processing device operatively connected to the memory, wherein the processing device is to execute instructions from the memory to perform a method to: receive intraoral scan data of a dentition of a patient; segment the intraoral scan data into a plurality of oral structures, wherein the plurality of oral structures comprises at least a tooth in the dentition of the patient, a gingiva, and a representation of an intersection between a first portion of the tooth and a second portion of the tooth; determine a gingival recession measurement indicative of a distance between the gingiva and the intersection; and provide, to a user device, the gingival recession measurement.

A second implementation may further extend the first implementation. In the second implementation, the method further comprises: identifying a shape of a line separating the gingiva from the first portion of the tooth along a facial surface of the tooth, wherein the first portion of the tooth represents a cementum of the tooth.

A third implementation may further extend the first and/or second implementation. In the third implementation, the method further comprises: determining a treatment recommendation based at least in part of the shape of the line; and providing, to the user device, the treatment recommendation.

A fourth implementation may further extend the first through third implementations. In the fourth implementation, the method further comprises: identifying, based on the shape of the line, a cause of gingival recession for the patient, wherein the treatment recommendation is further based at least in part on the cause of the gingival recession.

A fifth implementation may further extend the first through fourth implementations. In the fifth implementation, identifying the shape of the line further comprises: providing, as input to a trained machine learning model, the intraoral scan data; and receiving, as output from the trained machine learning model, the shape of the line separating the gingiva from the first portion of the tooth along the facial surface of the tooth.

A sixth implementation may further extend the first through fifth implementations. In the sixth implementation, identifying the shape of the line further comprises: measuring a second distance between the gingiva and the intersection at a plurality of points along the intersection; and responsive to determining that a difference between the second distance at two consecutive points of the plurality of points satisfies a criterion, identifying the shape of the line as a first shape corresponding to the criterion.

A seventh implementation may further extend the first through sixth implementations. In the seventh implementation, the method further comprises: receiving an occlusion data associated with the patient, wherein the treatment recommendation is further based at least in part the occlusion data associated with the patient.

An eighth implementation may further extend the first through seventh implementations. In the eighth implementation, segmenting the intraoral scan data into a plurality of oral structures comprises: providing, as input to a trained machine learning model, the intraoral scan data; and receiving, as output from the trained machine learning model, segmented scan data indicating the plurality of oral structures.

A ninth implementation may further extend the first through eighth implementations. In the ninth implementation, the gingival recession measurement represents an apical measurement between the gingiva and the intersection between the first portion of the tooth and the second portion of the tooth.

A tenth implementation may further extend the first through ninth implementations. In the tenth implementation, the method further comprises: maintaining a datastore comprising a plurality of gingival recession measurements for the patient, wherein the plurality of gingival recession measurements for the patient are generated over a period of time, and wherein the plurality of gingival recession measurements comprises the gingival recession measurement indicative the distance between the gingiva and the first portion of the tooth; and determining, based on the plurality of gingival recession measurements for the patient, a gingival recession progression over the period of time, wherein the treatment recommendation is further based on at least the gingival recession progression over the period of time.

An eleventh implementation may further extend the first through tenth implementations. In the eleventh implementation, the intraoral scan data comprises one or more intraoral scans generated by an intraoral scanner.

A twelfth implementation may further extend the first through eleventh implementations. In the twelfth implementation, the intraoral scan data comprises a three-dimensional model of the dentition of the patient generated from a plurality of intraoral scans.

A thirteenth implementation may further extend the first through twelfth implementations. In the thirteenth implementation, the intraoral scan data comprises three-dimensional scan data, two-dimensional near infrared scan data, and two-dimensional color scan data, and wherein at least two of the three-dimensional scan data, the two-dimensional near infrared scan data and the two-dimensional color scan data are processed together to determine the gingival recession measurement.

A fourteenth implementation may further extend the first through thirteenth implementations. In the fourteenth implementation, the method further comprises: generating a three-dimensional (3D) model of the dentition of the patient based on the three-dimensional scan data, the two-dimensional near infrared scan data, or the two-dimensional color scan data; and providing, to the user device, the 3D model of the dentition of the patient together with at least one of the gingival recession measurement or the treatment recommendation.

A fifteenth implementation may further extend the first through fourteenth implementations. In the fifteenth implementation, the representation of the intersection between the first portion of the tooth and the second portion of the tooth comprises a cementoenamel junction (CEJ) of the tooth.

A sixteenth implementation may further extend the first through fifteenth implementations. In the sixteenth implementation, the first portion of the tooth comprises enamel of the tooth, wherein the second portion of the tooth comprises cementum of the tooth, and wherein the intersection of the first portion of the tooth and the second portion of the tooth comprises a cementoenamel junction (CEJ) of the tooth.

A seventeenth implementation may further extend the first through sixteenth implementations. In the seventeenth implementation, determining the gingival recession measurement comprises: providing, as input to a trained machine learning model, the segmented intraoral scan data; and receiving, as output from the trained machine learning model, the measurement indicative of the distance between the gingiva and the intersection.

An eighteenth implementation may further extend the first through seventeenth implementations. In an eighteenth implementation, determining the gingival recession measurement comprises: comparing the distance between the gingiva and the intersection at a plurality of points along the intersection, wherein the gingival recession measurement comprises a highest distance.

In nineteenth through thirty-sixth implementations, a method comprises any of the first through eighteenth implementations.

In thirty-seventh through fifty-fourth implementations, a non-transitory computer-readable storage medium includes instructions that, when executed by a processing device, cause the processing device to perform any of the first through eighteenth implementations.

In a fifty-fifth implementation, a method comprises: receiving data representing a potential for temporomandibular disorder (TMD) of a patient; processing the data to identify an indicator of the TMD; identifying a treatment recommendation based on the indicator of the TMD; and providing the treatment recommendation for display on a user device.

A fifty-sixth implementation may further extend the fifty-fifth implementation. In the fifty-sixth implementation, the data comprises at least one of audio data representing a sound of the potential for TMD of the patient, video data representing a video recording of the patient, or a cone-beam computed tomography (CBCT) scan of the patient.

A fifty-seventh implementation may further extend the fifty-fifth first and/or fifty-sixth implementation. In the fifty-seventh implementation, the video recording is captured as the patient performs at least one of opening, closing, lateral, or protrusive jaw movements.

A fifty-eighth implementation may further extend the fifty-fifth through fifty-seventh implementations. In the fifty-eighth implementation, the audio data is captured while the patient performs at least one of opening, closing, lateral, or protrusive jaw movements.

A fifty-ninth implementation may further extend the fifty-fifth through fifty-eighth implementations. In the fifty-ninth implementation, the CBCT scan is of a jaw of the patient, and the CBCT scan represents the jaw of the patient in one of an open-jaw position or a closed-jaw position.

A sixtieth implementation may further extend the fifty-fifth through fifty-ninth implementations. In the sixtieth implementation, the treatment recommendation comprises an aligner treatment that accommodates the TMD, wherein the aligner treatment comprises an aligner that is designed to reduce one or more symptoms of the TMD.

A sixty-first implementation may further extend the fifty-fifth through sixtieth implementations. In the sixty-first implementation, the treatment recommendation comprises at least one of: not implementing an aligner treatment, stopping an aligner treatment, or slowing down an aligner treatment.

An sixty-second implementation may further extend the fifty-fifth through sixty-first implementations. In the sixty-second implementation, the method further comprises fabricating an appliance based on the indicator of the TMD.

A sixty-third implementation may further extend the fifty-fifth through sixty-second implementations. In the sixty-third implementation, the appliance comprises a 3D-printed appliance to correct the TMD.

A sixty-fourth implementation may further extend the fifty-fifth through sixty-third implementations. In the sixty-fourth implementation, the appliance comprises a 3D-printed appliance to concurrently treat the TMD and orthodontically move teeth.

An sixty-fifth implementation may further extend the fifty-fifth through sixty-fourth implementations. In the sixty-fifth implementation, the treatment recommendation comprises an appliance to correct the TMD.

A sixty-sixth implementation may further extend the fifty-fifth through sixty-fifth implementations. In the sixty-sixth implementation, processing the data to identify the indicator of the TMD comprises: providing the data as input to a machine learning model that is trained to output a value representing a likelihood of the TMD.

In a sixty-seventh implementation, a method comprises: receiving audio data representing a sound of a potential for temporomandibular disorder (TMD) of a patient; processing the audio data to identify an indicator of the TMD; identifying a treatment recommendation based on the indicator of the TMD; and providing the treatment recommendation for display on a user device.

A sixty-eighth implementation may further extend the sixty-seventh implementation. In the sixty-eighth implementation, the audio data is captured while the patient performs at least one of opening, closing, lateral, or protrusive jaw movements.

A sixty-ninth implementation may further extend the sixty-seventh and/or sixty-eighth implementations. In the sixty-ninth implementation, processing the audio data to identify the indicator of the TMD comprises providing the audio data as input to a machine learning model that is trained to output a value representing a likelihood of the TMD.

A seventieth implementation may further extend the sixty-seventh through sixty-ninth implementations. In the thirty-sixth implementation, processing the audio data to identify the indicator of the TMD comprises classifying the audio data using one or more digital signal processing techniques.

A seventy-first implementation may further extend the sixty-seventh through seventieth implementations. In the seventy-first implementation, the method further comprises: receiving a recording of the sound of the potential for the TMD, wherein the recording comprises analog audio signals; and converting the recording of the sound of the potential for the TMD to a digital signal, wherein the audio data comprises the digital signal.

A seventy-second implementation may further extend the sixty-seventh through seventy-first implementations. In the seventy-second implementation, the method further comprises: performing a preprocessing of the audio data, wherein the preprocessing comprises at least one of: filtering the audio data to remove background noise; extracting frequency data from the audio data, wherein the frequency data comprises a first frequency range corresponding to the sound of the TMD and a second frequency range not corresponding to the sound of the TMD; amplifying the first frequency range; reducing the second frequency range; or converting the audio data to a spectrogram representing the frequency data over time.

A seventy-third implementation may further extend the sixty-seventh through seventy-second implementations. In the seventy-third implementation, the method further comprises: filtering the audio data to remove background noise prior to identifying the indicator of the TMD.

A seventy-fourth implementation may further extend the sixty-seventh through seventy-third implementations. In the seventy-fourth implementation, the method further comprises: extracting frequency data from the audio data, wherein the frequency data comprises a first frequency range corresponding to the sound of the TMD and a second frequency range not corresponding to the sound of the TMD; and amplifying the first frequency range prior to identifying the indicator of the TMD.

A seventy-fifth implementation may further extend the sixty-seventh through seventy-fourth implementations. In the seventy-fifth implementation, the method further comprises: extracting frequency data from the audio data, wherein the frequency data comprises a first frequency range corresponding to the sound of the TMD and a second frequency range not corresponding to the sound of the TMD; and reducing or removing the second frequency range prior to identifying the indicator of the TMD.

A seventy-sixth implementation may further extend the sixty-seventh through seventy-fifth implementations. In the seventy-sixth implementation, the method further comprises: converting the audio data to a spectrogram representing frequency data over time; and processing the spectrogram using a trained machine learning model, wherein the trained machine learning model outputs the indicator of the TMD.

A seventy-seventh implementation may further extend the sixty-seventh through seventy-sixth implementations. In the seventy-seventh implementation, identifying the treatment recommendation corresponding to the indicator of the TMD comprises: receiving one or more responses to a patient questionnaire; analyzing the one or more responses to identify an additional indicator of the TMD; and determining that the patient has the TMD based on a combination of the indicator of the TMD and the additional indicator of the TMD.

A seventy-eighth implementation may further extend the sixty-seventh through seventy-seventh implementations. In the seventy-eighth implementation, the method further comprises: receiving video data representing a video recording of the patient, wherein the video recording is captured as the patient performs at least one opening, closing, lateral, or protrusive jaw movements; processing the video data to identify a second indicator of the TMD; and identifying the treatment recommendation further based on the second indicator.

A seventy-ninth implementation may further extend the sixty-seventh through seventy-eighth implementations. In the seventy-ninth implementation, the method further comprises: receiving a cone-beam computed tomography (CBCT) scan of the patient; analyzing the CBCT scan to identify a third indicator of the TMD; and identifying the treatment recommendation further based on the third indicator.

A eightieth implementation may further extend the seventy-third through seventy-ninth implementations. In the eightieth implementation, the method further comprises: receiving video data representing a video recording of the patient, wherein the video recording is captured as the patient performs at least one opening, closing, lateral, or protrusive jaw movements; processing the video data to identify a second indicator of the TMD; receiving a cone-beam computed tomography (CBCT) scan of the patient; analyzing the CBCT scan to identify a third indicator of the TMD; and identifying the treatment recommendation further based on the second indicator and the third indicator.

In an eighty-first implementation, a system comprises a memory and a processing device operatively connected to the memory, wherein the processing device is to execute instructions from the memory to perform a method comprising: receiving video data representing a video recording of a patient with a potential for temporomandibular disorder (TMD); processing the video data to identify an indicator of the TMD; identifying a treatment recommendation based on the indicator of the TMD; and providing the treatment recommendation for display on a user device.

A eighty-second implementation may further extend the eighty-first implementation. In the eighty-first implementation, the video data is captured while the patient performs at least one of opening, closing, lateral, or protrusive jaw movements.

An eighty-third implementation may further extend the eighty-first and/or eighty-second implementations. In the eighty-third implementation, processing the video data to identify the indicator of the TMD comprises: segmenting each frame of the video data into a plurality of features; identifying, in each frame, a first feature of a head of the patient and a second feature of the head of the patient; measuring, for each frame, a distance between the first feature of the head and the second feature of the head; determining a difference between a first distance for a first frame to a second distance for a second frame; and responsive to determining that the difference satisfies a criterion, setting the indicator to indicate presence of the TMD.

An eighty-fourth implementation may further extend the eighty-first through the eighty-third implementations. In the eighty-fourth implementation, the first frame and the second frame are consecutive frames.

An eighty-fifth implementation may further extend the eighty-first through the eighty-fourth implementations. In the eighty-fifth implementation, the method further comprises: stabilizing the video data to one or more fixed points of a head of the patient.

An eighty-sixth implementation may further extend the eighty-first through the eighty-fifth implementations. In the eighty-sixth implementation, processing the video data to identify the indicator of the TMD comprises: providing the video data as input to a machine learning model that is trained to output a value representing a likelihood of the TMD.

An eighty-seventh implementation may further extend the eighty-first through the eighty-sixth implementations. In the eighty-seventh implementation, identifying the treatment recommendation corresponding to the indicator of the TMD comprises: receiving one or more responses to a patient questionnaire; analyzing the one or more responses to identify an additional indicator of the TMD; and determining that the patient has the TMD based on a combination of the indicator of the TMD and the additional indicator of the TMD.

An eighty-eighth implementation may further extend the eighty-first through the eighty-seventh implementations. In the eighty-eighth implementation, the method further comprises: receiving audio data representing an audio recording of the patient, wherein the audio recording is captured as the patient performs at least one of opening, closing, lateral, or protrusive jaw movements; processing the audio data to identify a second indicator of the TMD; and identifying the treatment recommendation further based on the second indicator.

An eighty-ninth implementation may further extend the eighty-first through the eighty-eighth implementations. In the eighty-ninth implementation, the method further comprises: receiving a cone-beam computed tomography (CBCT) scan of the patient; analyzing the CBCT scan to identify a third indicator of the TMD; and identifying the treatment recommendation further based on the third indicator.

A ninetieth implementation may further extend the eighty-first through the sixty-ninth implementations. In the ninetieth implementation, the method further comprises: receiving audio data representing an audio recording of the patient, wherein the audio recording is captured as the patient performs at least one of opening, closing, lateral, or protrusive jaw movements; processing the audio data to identify a second indicator of the TMD; receiving a cone-beam computed tomography (CBCT) scan of the patient; analyzing the CBCT scan to identify a third indicator of the TMD; and identifying the treatment recommendation further based on the second indicator and the third indicator.

In a ninety-first implementation, a non-transitory computer-readable storage medium includes instructions that, when executed by a processing device, cause the processing device to perform operations comprises: receiving a cone-beam computed tomography (CBCT) scan of a jaw of a patient; processing the CBCT scan to identify an indicator of temporomandibular disorder (TMD) for the patient; identifying a treatment recommendation based on the indicator of the TMD; and providing the treatment recommendation for display on a user device.

A ninety-second implementation may further extend the ninety-first implementation. In the ninety-second implementation, the CBCT scan represents the jaw of the patient in one of an open-jaw position or a closed-jaw position.

A ninety-third implementation may further extend the ninety-first and/or the ninety-second implementations. In the ninety-third implementation, processing the CBCT scan to identify the indicator of the TMD for the patient comprises: segmenting the CBCT scan to identify a first region of the jaw of the patient and a second region of the jaw of the patient; identifying a first bone density represented in the first region and a second bone density represented in the second region; determining a difference between the first bone density and the second bone density; and responsive to determining that the difference satisfies a criterion, identifying a presence of the TMD in the patient.

A ninety-fourth implementation may further extend the ninety-first through the ninety-third implementations. In the ninety-fourth implementation, processing the CBCT scan to identify the indicator of the TMD for the patient comprises: providing the CBCT scan as input to a machine learning model that is trained to output a value representing a likelihood of the TMD.

A ninety-fifth implementation may further extend the ninety-first through the ninety-fourth implementations. In the ninety-fifth implementation, the operations further comprise: identifying a third region of the jaw of the patient; comparing a position of a first portion of the third region to a second portion of the third region; determining, based on the comparison, that the position of the first portion is abnormal; and responsive to determining that the position of the first portion is abnormal, identifying a presence of the TMD in the patient.

A ninety-sixth implementation may further extend the ninety-first through the ninety-fifth implementations. In the ninety-sixth implementation, identifying the treatment recommendation corresponding to the indicator of the TMD comprises: receiving one or more responses to a patient questionnaire; analyzing the one or more responses to identify an additional indicator of the TMD; and determining that the patient has the TMD based on a combination of the indicator of the TMD and the additional indicator of the TMD.

A ninety-seventh implementation may further extend the ninety-first through the ninety-sixth implementations. In the ninety-seventh implementation, the operations further comprise: receiving video data representing a video recording of the patient, wherein the video recording is captured as the patient performs at least one opening, closing, lateral, or protrusive jaw movements; processing the video data to identify a second indicator of the TMD; and identifying the treatment recommendation further based on the second indicator.

A ninety-eighth implementation may further extend the ninety-first through the ninety-seventh implementations. In the ninety-eighth implementation, the operations further comprise: receiving audio data representing an audio recording of the patient, wherein the audio recording is captured as the patient performs at least one of opening, closing, lateral, or protrusive jaw movements; processing the audio data to identify a third indicator of the TMD; and identifying the treatment recommendation further based on the third indicator.

A ninety-ninth implementation may further extend the ninety-first through the ninety-eighth implementations. In the ninety-ninth implementation, the operations further comprise: receiving video data representing a video recording of the patient, wherein the video recording is captured as the patient performs at least one opening, closing, lateral, or protrusive jaw movements; processing the video data to identify a second indicator of the TMD; receiving audio data representing an audio recording of the patient, wherein the audio recording is captured as the patient performs at least one of opening, closing, lateral, or protrusive jaw movements; processing the audio data to identify a third indicator of the TMD; and identifying the treatment recommendation further based on the second indicator and the third indicator.

### BRIEF DESCRIPTION OF THE DRAWINGS

Aspects and embodiments of the present disclosure will be understood more fully from the detailed description given below and from the accompanying drawings of various aspects and embodiments of the disclosure, which, however, should not be taken to limit the disclosure to the specific aspects or embodiments, but are for explanation and understanding only.

FIG. 1 shows a block diagram of an example system for dental diagnoses, in accordance with some embodiments of the present disclosure.

FIG. 2 shows a block diagram of an example system for 3D scan based gingival recession measurement and categorization, in accordance with some embodiments of the present disclosure.

FIG. 3 shows a block diagram of an example system for detecting and/or assessing TMD, in accordance with some embodiments of the present disclosure.

FIG. 4 illustrates a flow diagram of an example method for measuring and categorizing gingival recession of a patient, in accordance with some embodiments of the present disclosure.

FIG. 5A illustrates a flow diagram of an example method for measuring gingival recession, in accordance with some embodiments of the present disclosure.

FIG. 5B illustrates a flow diagram of an example method for categorizing gingival recession, in accordance with some embodiments of the present disclosure.

FIG. 6 illustrates workflows for training one or more machine learning models to perform gingival recession measurement and categorization, in accordance with some embodiments of the present disclosure.

FIG. 7 illustrates U-shaped and V-shaped gingival recession, in accordance with some embodiments of the present disclosure.

FIG. 8 shows a block diagram of an example TMD diagnostics system, in accordance with some embodiments of the present disclosure.

FIG. 9 illustrates a flow diagram of an example method for detecting and/or assessing TMD in a patient, in accordance with some embodiments of the present disclosure.

FIG. 10 illustrates a flow diagram of an example method for detecting and/or assessing TMD in a patient using audio data, in accordance with some embodiments of the present disclosure.

FIG. 11 illustrates a flow diagram of an example method for detecting and/or assessing TMD in a patient using video data, in accordance with some embodiments of the present disclosure.

FIG. 12 illustrates a flow diagram of an example method for detecting and/or assessing TMD in a patient using scan data, in accordance with some embodiments of the present disclosure.

FIG. 13 illustrates workflows for training one or more machine learning models to perform TMD detection, assessment, and/or diagnosis, in accordance with some embodiments of the present disclosure.

FIG. 14 illustrates example sagittal views of CT images of condyles representing examples of non-osteoarthritic or indeterminate osseous changes, in accordance with some embodiments of the present disclosure.

FIG. 15 illustrates examples of sagittal views of CT images of condyles representing osseous changes, in accordance with some embodiments of the present disclosure.

FIG. 16 illustrates example axially corrected coronal view of CT images of condyles representing examples of osseous changes, in accordance with some embodiments of the present disclosure.

FIG. 17 illustrates a block diagram of an example computing device, in accordance with some embodiments of the present disclosure.

FIG. 18 illustrates a flow diagram of an example data flow for detecting, predicting, diagnosing, and reporting on oral conditions and/or oral health problems, in accordance with some embodiments of the present disclosure.

### DETAILED DESCRIPTION

Described herein are embodiments of performing dental diagnoses, including intraoral scan-based gingival recession measurement and categorization, and assessment of temporomandibular disorder (TMD). Gingival recession can be described as the displacement of the gingival margin apical to the cementoenamel junction (CEJ). The gingival margin is the most coronal edge of the gingiva. The cementoenamel junction is where the enamel joins the cementum of teeth. Early detection and intervention may be used in preventing the progression of gingival recession and averting more severe dental problems. Regular dental check-ups and consistent monitoring of the condition, including appropriate measurements and characterization of patient conditions, may be used to effectively manage and ameliorate this condition.

Assessing and characterizing gingival recession typically involves manually measuring the distance between the CEJ and the gingival margin. This manual measurement is conventionally performed using a periodontal probe marked with distance measures. The measurement reflects the exposure of the root cementum. This traditional method of manually assessing gingival recession presents several challenges and limitations. For example, the markings on the periodontal probe may be difficult to read, leading to inaccurate measurements. In addition, the process of manually recording the gingival recession measurement for each tooth can be time-consuming, both for the patient and the dental professional. This can lead to longer appointment times and reduced efficiency within the dental practice. Furthermore, the accuracy of the measurements taken with a metal probe can vary significantly between dental practitioners. There is variability within each dental practitioners' measurement approach, and measurements can depend on the technique and pressure applied by different dentists, dental hygienists, or technicians. Once gingival recession has been identified in a patient, a dentist may monitor the progression of the recession over time. The variability within each dental professional's approach can lead to inconsistent and unreliable data, which may affect the diagnosis and treatment plan for the patient.

Additionally, the diagnosis of TMD can involve a multifaceted approach that includes patient history, clinical examination, and/or diagnostic imaging. The patient history can include gathering pain characteristics, functional limitations, headache history, jaw locking, and/or auditory symptoms from the patient. For example, a dental professional can ask the patient to describe the location and severity of pain surrounding the TMJ; the frequency, type, and/or intensity of headaches associated with TMD; difficulties in jaw movements (including opening, closing, lateral, and/or protrusive movements); and/or auditory sounds, such as clicking, popping, snapping, crepitus (grating sounds), etc., during jaw movements. During the clinical examination, a dental professional can perform a physical examination that includes palpation, measuring the range of motion of the jaw movements, listening for joint sounds during jaw movements, and/or evaluation the alignment of the teeth. Diagnostic imaging can include, for example, panoramic x-rays, MRIs, CBCT, and/or computed tomography (CT) scans. The dental professional can use a combination of these factors to diagnose the TMD, and to identify a probable cause.

Diagnosing TMD using these techniques presents several challenges due to the complexity of the disorder, the variability of its symptoms, and the variation in clinical expertise of the dental professionals making the diagnosis. For example, the symptoms of TMD can overlap with other conditions, which can make diagnosing TMD subjective to the dental professional making the diagnosis.

Accordingly, aspects and implementations of the present disclosure provide an integrated system for the automated analysis of dental clinical conditions, encompassing both gingival recession measurement and categorization, as well as TMD assessment. The systems and methods described herein can share a technological framework that leverages advanced data acquisition, image processing, and/or machine learning to deliver consistent, objective, and repeatable diagnostic outputs. Aspects of the present disclosure provide a generalized platform for dental condition analysis that can be extended to a wide range of oral health assessments.

In some embodiments, aspects and implementations of the present disclosure address the above challenges of dental treatment plans by providing systems and methods for consistently and accurately performing automatic measurement and categorization of gingival recession from 3D scanning of a patient. In some embodiments, a system is provided that may use and/or take an intraoral scan or a 3D model generated from intraoral scanning of a patient's dentition to measure and categorize gingival recession. The gingival recession may be automatically assessed at a point in time, such as during a patient visit. The systems and methods can be used in a repeatable process that can be used to track changes in gingival recession over time. For example, gingival recession may be automatically measured and assessed at multiple different patient visits at different times, and the various gingival recession measurements at the different times may be compared to determine or track gingival recession of the patient over time. Gingival recession changes may be positive or negative over time, meaning that the recession may be improved. For some patients, alignment and/or movement of the teeth could result in an improvement or worsening of gingival recession. As an example, inflammation of the gums could cause a temporary decrease in the measurement. However, the gingival recession may remain. If left untreated, one or more oral conditions (e.g., malocclusion, gum disease, etc.) could lead to continued progression of gingival recession over time. Thus, accurately tracking the gingival progression over time using implementations described herein may result in improved detection and treatment of gingival recession.

In some embodiments, in order to automatically measure and/or assess gingival recession for a patient, processing logic performs segmentation of an intraoral scan, of a 2D image associated with an intraoral scan and/or from a 3D model generated from a plurality of intraoral scans. In some embodiments, the segmentation can be performed in 3D (e.g., from an intraoral scan or from a 3D model). Alternatively, the 3D scan (or 3D model) can be projected into 2D, and segmentation can be performed in 2D. The resulting segmentation performed in 2D can then be back-projected onto the 3D scan or 3D model in embodiments. The intraoral scan (or other image data) can be segmented to identify tooth, gingiva, cementoenamel junction (CEJ), cementum, and/or enamel, for example. Once oral structures such as teeth, gingiva, CEJ, cementum and/or enamel are segmented, measurements may be made with respect to one or more of these oral structures. For example, the distance between the CEJ and the gingiva may be automatically measured to determine a gingival recession measurement.

In one embodiment, once the intraoral scan, 3D model or 2D image is segmented, the system can identify, for each tooth, the gingival line dividing the tooth from the gingiva. For teeth with gingival recession, the system can also identify the CEJ. The CEJ can be a segmented line or region, or can be identified as the boundary between the cementum and enamel. A visible CEJ indicates the presence of gingival recession.

For teeth with gingival recession, a measurement algorithm can identify the maximum distance between the gingiva and the CEJ along the facial surface of the tooth. In some embodiments, the measurement can be determined using a trained machine learning model that receives segmented scan data as input, and outputs a gingival recession measurement. In some embodiments, the measurement can be determined as the average of the difference between the gingiva and CEJ at various points along the surface of the tooth. In some embodiments, the measurement is recorded at number of locations along the tooth surface. For example, the measurement may be recorded at one edge of the tooth, at the middle of the tooth, and at an opposite edge of the tooth. The measurements may be automatically recorded in the patient's dental chart in order to track the patient's gingival recession over time. Due to the repeatable nature of the measurement process, aspects of the present disclosure enable for longitudinal data (i.e., measurements taken over time) that is a more accurate measure of gingival recession over time than gingival recession measurements manually performed by dentists, hygienists, and/or technicians.

In some embodiments, the gingival margin and the CEJ can be used to categorize the type of a detected gingival recession. Gingival recession can be categorized as “U” shaped or “V” shaped. The system can analyze the geometric shape formed by the gingival line to categorize the recession as “U” or “V” shaped in embodiments. In some embodiments, the categorization can be determined using geometric assessment of the various distances between the CEJ and the gingival margin along the tooth. In some embodiments, the categorization can be determine using a trained machine learning model that receives as input the segmented scan data, and outputs a classification of the gingival recession for each tooth (e.g., either as “U” shaped or “V” shaped). In some embodiments, a machine learning model may output one or more gingival recession measurements as well as a gingival recession classification and/or severity level. In embodiments, the severity level may be determined based on the one or more gingival recession measurements and/or on the gingival recession classification.

The system can use the categorization of the recession, optionally along with the patient's chart and/or occlusogram, to identify a potential root cause of a detected gingival recession in some embodiments. The system can optionally provide a treatment recommendation. The treatment recommendation may take into account the potential root cause in embodiments. In an example, a “U” shaped gingival recession is most often linked to an oral hygiene issue, and the system can provide a dental care recommendation to slow or stop progression of the recession if a U shaped gingival recession is detected. The dental care recommendation can include, for example, oral hygiene instructions and/or a periodontal treatment. In an example, a “V” shaped gingival recession can be caused by occlusal trauma within the mouth. The system can analyze the 3D geometric surface of the tooth near the CEJ to identify an abfraction (e.g., tooth damage or wear along the cervical margin due to mechanical forces, and not caused by decay). The system can analyze the 3D geometric surface of the tooth near the occlusal and/or incisal tooth wear to detect any potential signs of occlusal trauma, including e.g. a chip, a crack, a fracture, and/or wear of the tooth or restoration (e.g., a flattened surface, exposed dentin, etc.). The system can also use the patient's occlusogram to identify areas of heavy collision (e.g., tooth grinding, bruxism, etc.) within the mouth. The abfraction and/or the occlusal trauma within the mouth is likely to have caused the “V” shaped gingival recession. Thus, the system can recommend an orthodontic treatment (e.g., the use of aligners) to correct the malocclusion and support the patient's dental health. In some embodiments, by tracking the progression of gingival recession over time during orthodontic care, the system can recommend to adjust an existing orthodontic treatment plan to stop further progression the gingival recession, and/or to attempt to improve the gingival recession.

In some embodiments, the gingival recession measurement, categorization, and/or treatment recommendation can be provided to a user device. This information can be presented to a patient using the intraoral scan, the 3D model of the patient's dental arch, 2D images of the patient's dental arch, a radiograph of the patient's teeth, etc., showing abfractions and/or the gingival recession, along with the patient's occlusogram showing the areas of heavy collision, to educate the patient on their recommended treatment.

Embodiments described herein provide for an improved method and apparatus for performing dental diagnoses (e.g., measuring gingival recession) in a manger that is that is patient-friendly, time-efficient, and capable of providing consistent and accurate measurements, thereby enhancing the overall quality of dental care and patient experience. Such improvements in measuring and categorization gingival recession are likely to result in increased patient satisfaction as well as improved diagnosis and treatment of gingival recession. Advantages of the present disclosure and embodiments discussed herein include a more accurate method of detecting, diagnosing, and treating gingival recession. The automatic measurement and categorization of gingival recession using intraoral scan data can result in a more accurate detection and long-term monitoring of gingiva recession, avoiding the human error that is currently inevitable with manually measuring and/or categorizing gingival recession.

In some embodiments, aspects and implementations of the present disclosure address challenges of diagnosing and treating TMD by providing systems and methods for a standardized digital assessing of temporomandibular disorder (TMD) using audio and/or video data of a patient's jaw movements, and/or CBCT scan data of a patient's jaw. The systems and methods described herein use acoustic processing, video-based motion assessment, and/or a CBCT scan, optionally combined with a patient questionnaire, to detect, assess, and/or diagnose TMD. In some embodiments, the systems and methods described herein can identify and/or provide a treatment plan to address or correct the detected TMD. The systems and methods described herein can be implemented on any computing device, such as a mobile device, personal computing device, or server device, or combination thereof. The systems and methods described herein enable laypeople and/or technicians to screen for TMD, and allow for non-radiological assessment of TMD by doctors (e.g., dentists, general practitioners, etc.).

In some embodiments, the patient questionnaire may be the entry point to assessing the presence of TMD. The questionnaire can allow a clinician to understand the type of pain the patient is feeling, including when and where the patient experiences pain and/or other TMD symptoms. In some embodiments, a patient can provide answers to the questionnaire independently, e.g., through an application running on their personal computer or mobile device. The answers to the questionnaire may lead to further TMD diagnostic measures, and/or may be combined with other patient data to diagnose TMD (patient history, prior diagnoses and treatments, etc.).

In some embodiments, TMD can be detected and/or assessed using acoustic technologies. A microphone can be placed on or near the TMJ as the patient opens and/or closes their jaw. The microphone can convert the sounds created during the opening and/or closing of the jaw into analog audio signals. These audio signals can then be converted to a digital signal (e.g., using an analog-to-digital converted (ADC)). The resulting digital signals can be captured and/or stored by a digital capture device. In some embodiments, the digital capture device can send the digital signals to an external processing device (e.g., for cloud-based processing). In some embodiments, the digital capture device can be a subsystem of the processing device performing the detection and assessment of the TMD. The processing device can optionally prepare the digital signals (e.g., by filtering the digital signals). The digital signals can be provided to an artificial intelligence (e.g., machine learning) model that is trained to output a likelihood of the recorded jaw having TMD and/or other information about a TMD. In some embodiments, the machine learning model can be a classifier machine learning model. The results of the assessment can be displayed on a display device, e.g., of the user device. In some embodiments, the display can be integrated with the processing device or the capture device. In some embodiments, the display can be a separate component. The display can optionally be formatted and returned to the individual (e.g., using the device), to their doctor (e.g., as an email or other document), and/or to another recipient (e.g., hygienist, technician).

In some embodiments, the microphone can be an external microphone that is connected to the ADC and the digital capture device. In some embodiments, the microphone can be a component integrated with additional components, including the ADC, the digital capture component, and/or the processing device. For example, the microphone can be part of a user's mobile phone, which can be held to the TMJ to capture the audio as the user opens and/or closes their jaw. In some embodiments, the microphone can be a custom device, such as a stethoscope microphone, that is optimized for listening and optionally recording sounds from the human body. In some embodiments, the microphone can be a component of an intraoral scanner. The microphone can be separately attached to the intraoral scanner, or can be built into the intraoral scanner (e.g., built into the base of a scan wand) in order to capture audio. For example, the audio can be captured while using the scanner for other diagnostic capabilities, such as during intraoral scanning.

In some embodiments, the processing device can be a standalone digital computer or mobile device (e.g., a laptop, a mobile phone, another mobile device). In some embodiments, the processing device can be custom hardware. In some embodiments, the processing device can be cloud-based computing resources, including compute instances, docker containers, serverless functions, etc. In some embodiments, the processing device can implement any number of digital preprocessing techniques, such as filtering to remove external noise, Fourier or wavelet processing to extract frequency information, and/or conversion to a spectrogram to assess frequencies over time.

In some embodiments, the artificial intelligence model that is used to assess the likelihood of TMD can be a classifier machine learning model. The ML model can receive, as input, either the raw or the optionally preprocessed audio signals. The ML model can be trained using, e.g., neural networks, deep learning, tree-based methods, linear/logistic classifiers, or any other method, to output a likelihood of the presence of TMD. In some embodiments, the processing device can implement classical digital signal processing techniques to assess TMD. Examples of classical digital signal processing techniques that may be used include matched filters, Wiener filters, Spectral methods, Bayesian methods, etc.

In some embodiments, TMD can be detected and/or assessed using a video. A video camera and/or video capture system can be used to collect video of the patient opening and/or closing their mouth. In some embodiments, video can be stabilized to a reference point. Frames of the video can be segmented and processed to identify (segment) the mandible and/or the open mouth. The degree to which the patient can open their mouth may be assessed in an absolute measure (e.g., millimeters of opening), in a relative measure of distance, and/or in a specific angle of opening. The presence and/or severity of TMD can be assessed by comparing the ability of the patient to open the mandible to one or more pre-determined thresholds. In addition to detecting and/or assessing the range of motion, sagittal video can identify when and/or where in the motion the patient's jaw “catches” or “pops.” The patient's jaw “catching” or “popping” can be seen in the video as a non-smooth or discontinuous motion in video frames. In some embodiments, the video can be captured simultaneously with the audio components to facilitate the acoustic assessment. In some embodiments, the video can be captured separately, in addition to or instead of the audio assessment.

In some embodiments, a processing device can implement a measurement system to measure the opening of the patient's jaw from video data (e.g., a video recording of the patient opening and/or closing their mouth). In some embodiments, the processing device can implement an artificial intelligence (e.g., machine learning) model to detect and/or assess TMD. The AI model can receive, as input, the segmented video data, and can provide, as output, a likelihood of the presence of TMD. The AI model can be trained to process a stream of images to detect motion indicative of TMD.

In some embodiments, TMD can be detected and/or assessed using a CBCT scan. The CBCT scan can be segmented into the mandible, the patient's teeth, and/or the TMJ's cartilage disc. In some embodiments, a processing device can identify the location of the disc. In some cases of TMD, a misplacement of the disc can be the cause of the patient's pain. In some cases, the patient may be experiencing degenerative joint disease, which manifests as deterioration of the bone. The deterioration can be reflected in either damaged bone or reduced bone density at the site of the TMJ, both of which can be detected from the CBCT.

In some embodiments, detection using CBCT can include a CBCT capture component, a CBCT segmentation component, and a CBCT assessment component. In some embodiments, the CBCT capture component can be a CBCT machine. In some embodiments, the CBCT capture component can be software that runs on a processing device to collect the raw data and reconstruct the 3D volumetric CBCT image. In some embodiments, the CBCT segmentation component can segment, at varying density levels, teeth, bone, and/or cartilage. In some embodiments, the CBCT assessment component can execute an AI model to identify misalignment of the jaw (e.g., dislocation of the disc), and/or relative bone density, and/or to otherwise identify TMD in CBCT scan data. The misalignment of the jaw and/or relative bone density can be indicators of TMD.

In some embodiments, the audio, video, and/or CBCT assessments can be performed individually or in combination. In some embodiments, the output of the assessment(s) can be combined with the patient questionnaire to detect, assess, and/or diagnose TMD, and/or to identify the cause of the TMD. For example, CBCT-scan data can be used to identify disc disorders (e.g., abnormal positioning of the disc in the TMJ) and/or bone destruction (e.g., due to a degenerative bone disease), and the patient questionnaire can be used to identify joint pain (e.g., arthralgia). The cause of the TMD can be identified as a disorder of the joint, which is identified based on combination of joint pain, disc disorder, and/or bone destruction can. As another example, the cause of the TMD can be identified as disorder of the masticatory muscles (e.g., muscles used for chewing), which be determined based on the location of the pain (e.g., pain located in one area that gets worse when pressure is applied (myalgia), pain that spreads beyond the point where it starts, or pain that is felt in an area of the body that is far away from where it started (myofascial pain without/with referral). The assessment, diagnosis, and/or identified cause of the TMD can be provided for display on a user device.

In some embodiments, systems and methods described herein can identify a treatment recommendation for the detected TMD. The treatment recommendation can be based on the severity of the detected TMD, the cause of the TMD, and/or the patient's medical history. For example, if a patient is undergoing orthodontic treatment when TMD symptoms first occur, the treatment recommendation may be to slow the progress of the orthodontic treatment, or to stop the orthodontic treatment if the severity of the symptoms of the TMD exceed a threshold. As another example, if a patient is a candidate for orthodontic treatment but presents with TMD symptoms, and the treatment recommendation may include not starting orthodontic treatment until the TMD has been addressed. As another example, the treatment recommendation may include fabricating an application based on the indication of TMD, e.g., to correct the TMD. In some embodiments, the appliance can be a 3D-printed appliance to correct TMD, or a 3D-printed appliance to concurrently treat the TMD and orthodontically move the teeth. The treatment recommendation can be based on a set of rules that take into account the patient's history (e.g., how long the patient has had symptoms, the severity of the symptoms, treatment history, etc.), the severity of the detected TMD, and/or the cause of the TMD. The treatment recommendation can be provided for display on a user device, e.g., along with the assessment, diagnosis, and/or identified cause of the TMD.

Embodiments described herein provide for an improved method and apparatus for performing dental diagnoses (e.g., detecting, assessing, and/or diagnosing TMD) in a manner that is patient-friendly, time-efficient, and capable of providing consistent and accurate indicators of TMD, thereby enhancing the overall quality of dental care and patient experience. Such improvements in detecting, assessing, and/or diagnosing TMD are likely to result in increased patient satisfaction as well as improved diagnosis and treatment of TMD. Advantages of the present disclosure and embodiments discussed herein include a more accurate method of detecting, assessing, and diagnosing TMD. The automatic TMD detection and assessment using audio, video, and/or scan data can result in a more accurate detection by doctors and laypersons, avoiding the human error that is currently inevitable with manually detection and assessment of TMD.

Both gingival recession and TMD represent complex, multifactorial conditions that can benefit from precise, longitudinal monitoring and clinical interpretation. Traditional diagnostic approaches for these conditions often rely on manual measurements, subjective clinical judgment, and/or disparate data sources, which can lead to variability in diagnosis and treatment planning. Aspects of the present disclosure address these challenges by employing digital data capture modalities (e.g., intraoral scans, 2D and/or 3D imaging, audio and/or video recordings, and/or CBCT scans, for example) combined with robust segmentation and analysis, which enable the automated identification, measurement, and/or categorization of oral structures and function indicators, supporting both point-in-time assessments and longitudinal tracking of disease progression. For example, aspects of the present discourse support integration with patient records and dental practice management systems, thus enabling the aggregation and analysis of longitudinal data across multiple visits. This can facilitate trend analysis, early detection of disease progression, and/or the ability to tailor treatment recommendations on a comprehensive view of the patient's oral health. The methods and systems described herein provide a unified approach to dental clinical condition analysis that improves diagnostic accuracy, consistency, and patient outcomes across a spectrum of dental health challenges.

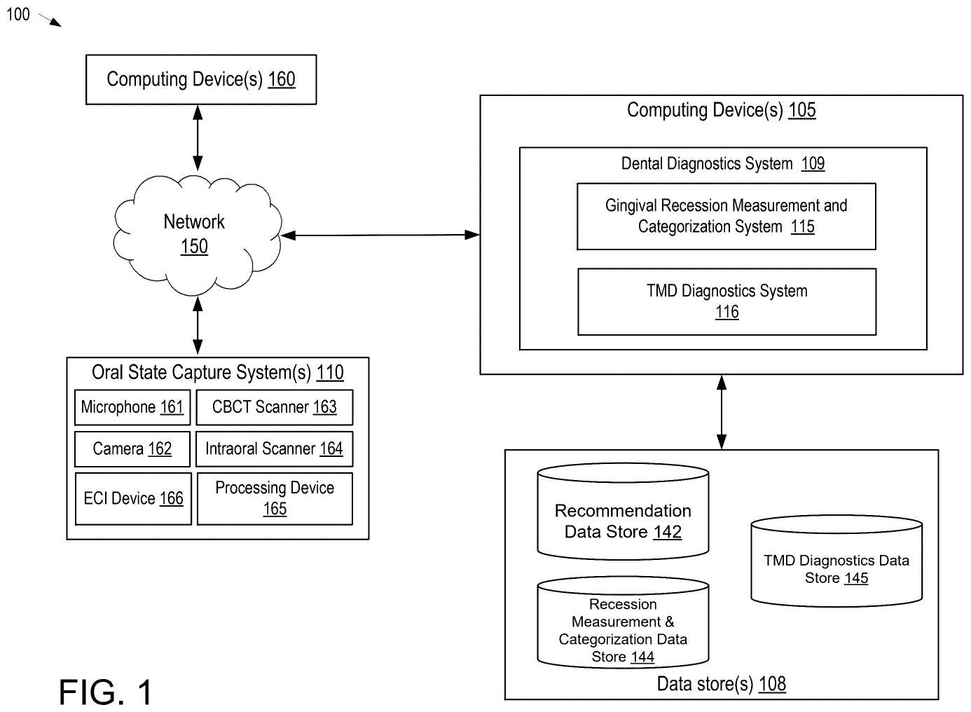

FIG. 1 illustrates a block diagram of an example system 100 for dental diagnoses, in accordance with some embodiments of the present disclosure. System 100 includes a computing device 105 that may be coupled to one or more computing devices 160, oral state capture system(s) 110, and/or a data store 108.

Computing devices 105 and/or 160 may each include a processing device, memory, secondary storage, one or more input devices (e.g., such as a keyboard, mouse, tablet, and so on), one or more output devices (e.g., a display, a printer, etc.), and/or other hardware components. Computing device 105 may be connected to a data store 108 either directly or via a network (e.g., network 150). The network 150 may be a local area network (LAN), a public wide area network (WAN) (e.g., the Internet), a private WAN (e.g., an intranet), or a combination thereof. The computing device 105 may additionally or alternatively be connected to computing device(s) 160 and/or oral state capture systems 110 via a network 150, which may be a local area network (LAN), a public wide area network (WAN) (e.g., the Internet), a private WAN (e.g., an intranet), or a combination thereof. In some embodiments, oral state capture system(s) 110 connect to computing device(s) 105 directly via a wired or wireless connection.

Data store 108 may be an internal data store, or an external data store that is connected to computing device 105 directly or via a network. Examples of network data stores include a storage area network (SAN), a network attached storage (NAS), and a storage service provided by a cloud computing service provider. Data store 108 may include a file system, a database, or other data storage arrangement. In some embodiments, data store 108 can include a recession measurement and categorization data store 144, TMD diagnostics data 145, and/or a recommendation data store 142.

In some embodiments, computing device 105 is a desktop computer, a laptop computer, a server computer, etc. located at a doctor office. In some embodiments, computing device 105 is a server computing device (e.g., of a data center) that may be accessed from client devices (e.g., client devices of doctors, patients, etc.). In some embodiments, computing device 105 is a virtual machine. For example, computing device 105 may be a virtual machine that runs in a cloud computing environment.

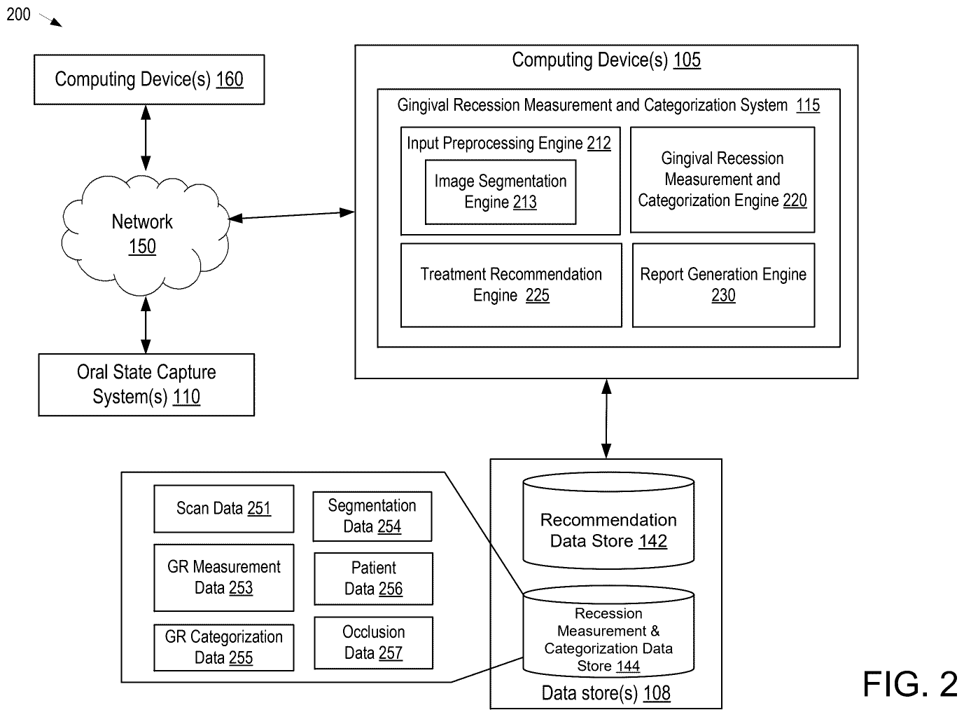

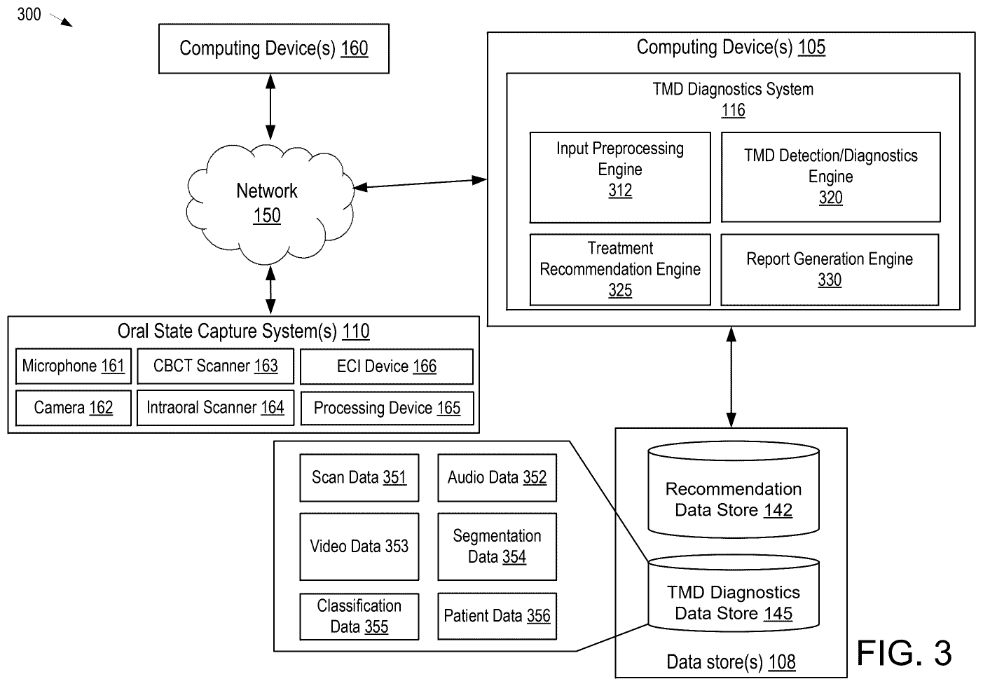

In some embodiments, computing device 105 includes a dental diagnostics system 109. The dental diagnostics system 109 can be a software program hosted by a device (e.g., computing device 105) to perform dental diagnoses for a patient. The diagnoses can include, for example, gingival recession and measurement, and/or TMD diagnostics. The dental diagnostics system 109 can include a gingival recession measurement and categorization system 115 and/or a TMD diagnostics system 116. The includes a gingival recession measurement and categorization system 115 is further described with respect to FIG. 2. The TMD diagnostics system 116 is further described with respect to FIG. 3.

In some embodiments, oral state capture system(s) 110 can include a microphone 161; a camera 162 (e.g., a video camera); a CBCT scanner 163 (and/or another imaging device, such as a CT scanner); an electronic compliance indicator (ECI) device 166 or other dental appliance to be worn by a patient that includes a microphone; an intraoral scanner 164; and/or optionally a computing device 165. The oral state capture system 110 can obtain audio, video, and/or image-based scans of a patient's dentition, jaw, and/or jaw movements. In some embodiments, the microphone 161, camera 162, CBCT scanner 163, intraoral scanner 164, the ECI device 166, and/or processing device 165 can be combined. For example, in some embodiments, the microphone can be built into the base of a scan wand of the intraoral scanner 164, which can be used to capture audio while a technician is using the intraoral scanner 164 for other diagnostic capabilities (e.g., to perform intraoral scanning). In some embodiments, oral state capture system 110 includes a dental appliance such as an aligner, palatal expander, etc. that includes a microphone. As another example, in some embodiments, the processing device 165 can be part of the intraoral scanner 164, CBCT scanner 163, and/or ECI device 166. In some embodiments, processing device 165 can be part of computing device 160, computing device 105, and/or a separate device (not shown), and the oral state capture system 110 can send captured data (e.g., scan data, audio data, and/or video data) for processing on a separate device. In one embodiment, oral state capture system 110 includes a patient or client device that can take 2D or 3D images, videos, and/or audio recordings of the patient's oral cavity in a non-clinical setting (e.g., at a patient's home).

In some embodiments, oral state capture system 110 may include a scanning system (e.g., CBCT scanner 163, intraoral scanner 164, and/or ECI device 166) that can perform scanning of the patient's mouth, jaw, head, oral cavity, and/or other area of the patient where the patient may be experiencing TMD-related symptoms. The scanning may be performed to generate a plurality of scans of the patient's jaw movements, which may be combined to generate a three dimensional (3D) model of a dentition and/or jaw of a patient. In some embodiments, oral state capture system 110 may include an imaging device, which may be a 2D or 3D imaging device, such as a digital camera, mobile phone, tablet computer, and so on. In some embodiments, the oral state capture system 110 may include a CBCT scanner 163, to capture CBCT scans of the patient's jaw. A CBCT scanner 163 is a type of x-ray machine that uses a cone-shaped x-ray beam to capture data about the patient's anatomy. The CBCT scanner 173 can generate multiple (e.g., 150-200) images from a variety of angles.

FIG. 2 illustrates a block diagram of an example system 200 for intraoral scan-based gingival recession measurement and categorization, in accordance with some embodiments of the present disclosure. System 200 includes a computing device 105 that may be coupled to one or more computing devices 160, oral state capture system(s) 110, and/or a data store 108. In some embodiments, computing device 160, oral state capture system 110, computing device 105, and/or data store 108 of FIG. 2 can perform the same function as computing device 160, oral state capture system 110, computing device 105, and/or data store 108 of FIG. 1.

Computing devices 105 and/or 160 may each include a processing device, memory, secondary storage, one or more input devices (e.g., such as a keyboard, mouse, tablet, and so on), one or more output devices (e.g., a display, a printer, etc.), and/or other hardware components. Computing device 105 may be connected to a data store 108 either directly or via a network (e.g., network 150). The network 150 may be a local area network (LAN), a public wide area network (WAN) (e.g., the Internet), a private WAN (e.g., an intranet), or a combination thereof. The computing device 105 may additionally or alternatively be connected to computing device(s) 160 and/or oral state capture systems 110 via a network 150, which may be a local area network (LAN), a public wide area network (WAN) (e.g., the Internet), a private WAN (e.g., an intranet), or a combination thereof. In some embodiments, oral state capture system(s) 110 connect to computing device(s) 105 directly via a wired or wireless connection.

Data store 108 may be an internal data store, or an external data store that is connected to computing device 105 directly or via a network. Examples of network data stores include a storage area network (SAN), a network attached storage (NAS), and a storage service provided by a cloud computing service provider. Data store 108 may include a file system, a database, or other data storage arrangement.

In some embodiments, data store 108 can include a recession measurement and categorization data store 144 and/or a recommendation data store 142. The recommendation data store 142 can include treatment recommendation rules, e.g., used by treatment recommendation engine 225 to identify treatment options. In some embodiments, the recommendation data store 142 can include the treatment recommendations and/or reports generated by treatment recommendation engine 225 and/or report generation engine 230. The recession measurement and categorization data store 144 can include scan data 251, gingival recession measurement data 253, gingival recession categorization data 255, segmentation data 254, patient data 256, and/or occlusion data 257. Patient data 256 can include a patient chart (e.g., patient dental chart), which can include longitudinal information about the patient's gingival recession history. The patient's gingival recession history may include, for example, intraoral scans, 2D images and/or 3D models of the patient's dental arch(es) generated at various points in time. The patient's gingival recession history may further or alternatively include gingival recession measurements, analyses, etc. generated from intraoral scans, 2D images and/or 3D models of the patient's dental arch(es) generated at various points in time. The patient's gingival recession history may additionally include doctor notes and/or observations input into the patient's record. Gingival recession changes may be positive or negative overtime, meaning that the recession may be improved. For some patients, alignment and/or movement of the teeth could result in an improvement or worsening of gingival recession. Inflammation of the gums could cause a temporary decrease in the measurement, but if left untreated, could lead to continued progression over time. Thus, an accurate log of the patient's gingival recession history can be used for the detection and treatment gingival recession.

In some embodiments, intraoral scan data 251 can include scan data generated by oral state capture system 110. In some embodiments, gingival recession measurement data 253 can include rules for measuring gingival recession of a patient's dentition. In some embodiments, gingival recession measurement data 253 can include measurements of gingival recession of a patient's dentition, as generated by gingival recession measurement and categorization engine 220. In some embodiments, gingival recession categorization data 255 can include rules for categorizing gingival recession. In some embodiments, gingival recession categorization data 255 can include categorization of the gingival recession of a patient's dentition, as generated by gingival recession measurement and categorization engine 220. In some embodiments, segmentation data 254 can include the segmented scan data, as generated by image segmentation engine 213. In some embodiments, occlusion data 257 can include occlusion data indicating occlusions of one or more teeth of a patient (e.g., as generated by oral state capture system 110). In some embodiments, the scan data 251, gingival recession measurement data 253, gingival recession categorization data 255, segmentation data 254, patient data 256, and/or occlusion data 257 can reference a patient identifier.

In some embodiments, oral state capture system 110 includes an intraoral scanning system that can perform intraoral scanning of the patient's oral cavity. The intraoral scanning may be performed to generate a plurality of intraoral scans of the patient's oral cavity, which may be combined to generate a three dimensional (3D) model of a dentition of a patient. Alternatively, oral state capture system 110 may include an imaging device, which may be a 2D or 3D imaging device, such as a digital camera, mobile phone, tablet computer, and so on. In one embodiment, oral state capture system 110 includes a patient or client device that can take 2D or 3D images of the patient's oral cavity in a non-clinical setting (e.g., at a patient's home).

In some embodiments, oral state capture system 110 is connect to data store(s) 108 either directly or via network 150. In some embodiments, oral state capture system 110 transmits image data (e.g., intraoral scan data, 2D images, 3D images, 3D models, etc.) to data store 108 for storage therein.

In one embodiment, oral state capture system 110 is an intraoral scanning system comprising a scanner (e.g., scanner 164 of FIG. 1) for obtaining intraoral scans (e.g., 3D data) of a patient's dentition and optionally a computing device. Alternatively, oral state capture system 110 may include an intraoral scanner, and computing device 105 may connect to the intraoral scanner to effectuate intraoral scanning.

In embodiments, computing device 105 or another computing device of oral state capture system 110 includes an intraoral scan application that processes intraoral scans generated by the intraoral scanner to generate 3D models of the patient's upper and/or lower dental arches.

Intraoral scanner may include a probe (e.g., a hand held probe) for optically capturing three-dimensional structures. The intraoral scanner may be used to perform an intraoral scan of a patient's oral cavity. An intraoral scan application running on computing device 105 (or on another computing device of oral state capture system 110) may communicate with the scanner to effectuate the intraoral scan. A result of the intraoral scan may be intraoral scan data 251 that may include one or more sets of intraoral scans, which may include intraoral images. Each intraoral scan may include a two-dimensional (2D) or 3D image that may include depth information (e.g., a height map) of a portion of a dental site. In embodiments, intraoral scans include x, y and z information. In one embodiment, the intraoral scanner generates numerous discrete (i.e., individual) intraoral scans.

In some embodiments, sets of discrete intraoral scans are merged into a smaller set of blended intraoral scans, where each blended scan is a combination of multiple discrete scans. The intraoral scan data 251 may include raw scans and/or blended scans, each of which may be referred to as intraoral scans (and in some instances as intraoral images). While scanning, the intraoral scanner may generate multiple (e.g., tens) of scans (e.g., height maps) per second (referred to as raw scans). In order to improve the quality of the data captured, a blending process may be used to combine a sequence of raw scans into a blended scan by some averaging process. Additionally, intraoral scanner may generate many scans per second. This may be too much data to process using a machine learning model in real time. Accordingly, groups of similar scans may be combined into the blended scans, and the blended scans may be input into one or more trained machine learning model. This may vastly reduce the computation resources used to process the intraoral scans without degrading quality. In one embodiment, each blended scan includes data from up to 20 raw scans, and further includes scans that differ by less than a threshold angular difference from one another and/or by less than a threshold positional difference from one another. Accordingly, some blended scans may include data from 20 scans, while other blended scans may include data from fewer than 20 scans. In one embodiment, the intraoral scan (which may be a blended scan) includes height values and intensity values for each pixel in the image.

Intraoral scan data 251 may also include color 2D images and/or images of particular wavelengths (e.g., near-infrared (NIRI) images, infrared images, ultraviolet images, etc.) of a dental site in embodiments. In embodiments, intraoral scanner alternates between generation of 3D intraoral scans and one or more types of 2D intraoral images (e.g., color images, NIRI images, etc.) during scanning. For example, one or more 2D color images may be generated between generation of a fourth and fifth intraoral scan. For example, some scanners may include multiple image sensors that generate different 2D color images of different regions of a patient's dental arch concurrently. These 2D color images may be stitched together to form a single color representation of a larger field of view that includes a combination of the fields of view of the multiple image sensors.

The scanner may transmit the intraoral scan data 251 to the computing device 105. Computing device 105 may store the intraoral scan data 251 in data store 108.

According to an example, a user (e.g., a practitioner) may subject a patient to intraoral scanning. In doing so, the user may apply an intraoral scanner to one or more patient intraoral locations. The scanning may be divided into one or more segments (also referred to as roles). As an example, the segments may include a lower dental arch of the patient, an upper dental arch of the patient, one or more preparation teeth of the patient (e.g., teeth of the patient to which a dental device such as a crown or other dental prosthetic will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with the scan being directed towards an interface area of the patient's upper and lower teeth). Via such scanner application, the intraoral scanner may provide intraoral scan data 251 to computing device 105 (or to another computing device of oral state capture system 110). The intraoral scan data 251 may be provided in the form of intraoral scan data sets, each of which may include 2D intraoral images (e.g., color 2D images) and/or 3D intraoral scans of particular teeth and/or regions of an intraoral site. In one embodiment, separate intraoral scan data sets are created for the maxillary arch, for the mandibular arch, for a patient bite, and/or for each preparation tooth. Alternatively, a single large intraoral scan data set is generated (e.g., for a mandibular and/or maxillary arch). Intraoral scans may be provided from the intraoral scanner to the computing device 105 (or other computing device) in the form of one or more points (e.g., one or more pixels and/or groups of pixels). For instance, the intraoral scanner may provide an intraoral scan as one or more point clouds. The intraoral scans may each comprise height information (e.g., a height map that indicates a depth for each pixel).

The manner in which the oral cavity of a patient is to be scanned may depend on the procedure to be applied thereto. For example, if an upper or lower denture is to be created, then a full scan of the mandibular or maxillary edentulous arches may be performed. In contrast, if a bridge is to be created, then just a portion of a total arch may be scanned which includes an edentulous region, the neighboring preparation teeth (e.g., abutment teeth) and the opposing arch and dentition. Alternatively, full scans of upper and/or lower dental arches may be performed if a bridge is to be created.

By way of non-limiting example, dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions such as malocclusions, temporomandibular joint disorder (TMD), gingival recession, tooth grinding, and so on. The term prosthodontic procedure refers, inter alia, to any procedure involving the oral cavity and directed to the design, manufacture or installation of a dental prosthesis at a dental site within the oral cavity (intraoral site), or a real or virtual model thereof, or directed to the design and preparation of the intraoral site to receive such a prosthesis. A prosthesis may include any restoration such as crowns, veneers, inlays, onlays, implants and bridges, for example, and any other artificial partial or complete denture. The term orthodontic procedure refers, inter alia, to any procedure involving the oral cavity and directed to the design, manufacture or installation of orthodontic elements at an intraoral site within the oral cavity, or a real or virtual model thereof, or directed to the design and preparation of the intraoral site to receive such orthodontic elements. These elements may be appliances including but not limited to brackets and wires, retainers, clear aligners, or functional appliances.

In embodiments, intraoral scanning may be performed on a patient's oral cavity during a visitation of a dental office. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental diagnostics, to generate a treatment plan, to determine progress of a treatment plan, and/or for other purposes. The intraoral scan data 251 generated from the intraoral scanning may include 3D scan data, 2D color images, NIR (near infrared) and/or infrared images, and/or ultraviolet images, of all or a portion of the upper jaw and/or lower jaw. The intraoral scan data 251 may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Intraoral scanners may work by moving the intraoral scanner inside a patient's mouth to capture all viewpoints of one or more tooth. During scanning, the intraoral scanner is calculating distances to solid surfaces in some embodiments. Each intraoral scan is overlapped algorithmically, or ‘stitched’, with the previous set of scans to generate a growing 3D surface. As such, each scan is associated with a rotation in space, or a projection, to how it fits into the 3D surface.

During intraoral scanning, an intraoral scan application (e.g., executing on computing device 105 or a computing device of oral state capture system 110) may register and stitch together two or more intraoral scans generated thus far from the intraoral scan session. In one embodiment, performing registration includes capturing 3D data of various points of a surface in multiple scans, and registering the scans by computing transformations between the scans. One or more 3D surfaces may be generated based on the registered and stitched together intraoral scans during the intraoral scanning. The one or more 3D surfaces may be output to a display so that a doctor or technician can view their scan progress thus far. As each new intraoral scan is captured and registered to previous intraoral scans and/or a 3D surface, the one or more 3D surfaces may be updated, and the updated 3D surface(s) may be output to the display. In embodiments, separate 3D surfaces are generated for the upper jaw and the lower jaw. This process may be performed in real time or near-real time to provide an updated view of the captured 3D surfaces during the intraoral scanning process.

When a scan session or a portion of a scan session associated with a particular scanning role (e.g., upper jaw role, lower jaw role, bite role, etc.) is complete (e.g., all scans for an intraoral site or dental site have been captured), the intraoral scan application may automatically generate a virtual 3D model of one or more scanned dental sites (e.g., of an upper jaw and a lower jaw). The final 3D model(s) may each be a set of 3D points and their connections with each other (i.e. a mesh). To generate a virtual 3D model, the intraoral scan application may register and stitch together the intraoral scans generated from the intraoral scan session that are associated with a particular scanning role. The registration performed at this stage may be more accurate than the registration performed during the capturing of the intraoral scans, and may take more time to complete than the registration performed during the capturing of the intraoral scans. In one embodiment, performing scan registration includes capturing 3D data of various points of a surface in multiple scans, and registering the scans by computing transformations between the scans. The 3D data may be projected into a 3D space of a 3D model to form a portion of the 3D model. The intraoral scans may be integrated into a common reference frame by applying appropriate transformations to points of each registered scan and projecting each scan into the 3D space.

In one embodiment, registration is performed for adjacent or overlapping intraoral scans (e.g., each successive frame of an intraoral video. Registration algorithms are carried out to register two adjacent or overlapping intraoral scans (e.g., two adjacent blended intraoral scans) and/or to register an intraoral scan with a 3D model, which essentially involves determination of the transformations which align one scan with the other scan and/or with the 3D model. Registration may involve identifying multiple points in each scan (e.g., point clouds) of a scan pair (or of a scan and the 3D model), surface fitting to the points, and using local searches around points to match points of the two scans (or of the scan and the 3D model). For example, the intraoral scan application may match points of one scan with the closest points interpolated on the surface of another scan, and iteratively minimize the distance between matched points. Other registration techniques may also be used.

The Intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D model). The intraoral scan application may integrate intraoral scans into a single virtual 3D model (or two virtual 3D models, one for each dental arch) by applying the appropriate determined transformations to each of the intraoral scans. Each transformation may include rotations about one to three axes and translations within one to three planes.

The generated virtual 3D model can include color information. In some embodiments, the scan data 251 can include color information, e.g., from 2D color images captured during the scanning process. The oral state capture system 110 can use the color information to add color texture to the 3D model(s).

Once virtual 3D model(s) of the patient's dental arches are generated, they may be stored in data store 108 as a portion of scan data 251 in embodiments.

In some embodiments, the oral state capture system 110 can use the scan data 251 to generate an occlosugram for the patient, which can represent the occlusions in the patient's dentition. An occlusion is the contact between teeth. An occlusogram can illustrate the occlusal clearance of one or more teeth of the patient. For example, the occlusogram can include an occlusal clearance color map that shows the contact relationship between the teeth on the patient's dental arches. The occlusogram can indicate portions of the teeth that have excessive force in the patient's occlusions, portions that have mild force in the patient's occlusions, and/or portions that have no occlusions. The occlusogram can be stored in occlusion data 257 in embodiments.

In some embodiments, computing device 105 is a desktop computer, a laptop computer, a server computer, etc. located at a doctor office. In some embodiments, computing device 105 is a server computing device (e.g., of a data center) that may be accessed from client devices (e.g., client devices of doctors, patients, etc.). In some embodiments, computing device 105 is a virtual machine. For example, computing device 105 may be a virtual machine that runs in a cloud computing environment.

In some embodiments, computing device 105 includes a gingival recession measurement and categorization system 115, which may include an input preprocessing engine 212, a gingival recession measurement and categorization engine 220, a treatment recommendation engine 225, and/or a report generation engine 230. Gingival recession measurement and categorization system 215 may include software, hardware and/or firmware configured to perform one or more operations with respect to measurement, analysis, prognosis and/or treatment of gingival recession and other dental conditions related to gingival recession.

In some embodiments, input preprocessing engine 212 can be a software program hosted by a device (e.g., computing device 105) to process intraoral scan data (e.g., intraoral scan data 251). Input preprocessing engine 212 can include an image segmentation engine 213. Input preprocessing engine 212 may perform one or more operations on scan data 251 to prepare the scan data 251 for analysis of gingival recession. Input preprocessing engine 212 may perform operations such as cropping, image enhancement (e.g., to sharpen an image), segmentation, and/or other operations. In some embodiments, if scan data 251 does not include a 3D model of a dental arch (e.g., includes 2D images or intraoral scans but no 3D models of dental arches), input preprocessing engine 212 may process the 2D images and/or intraoral scans to generate one or more 3D models (e.g., as discussed above). In some embodiments, 3D models may be generated from 2D images (e.g., such as those taken by a patient device such as a patient's mobile phone).

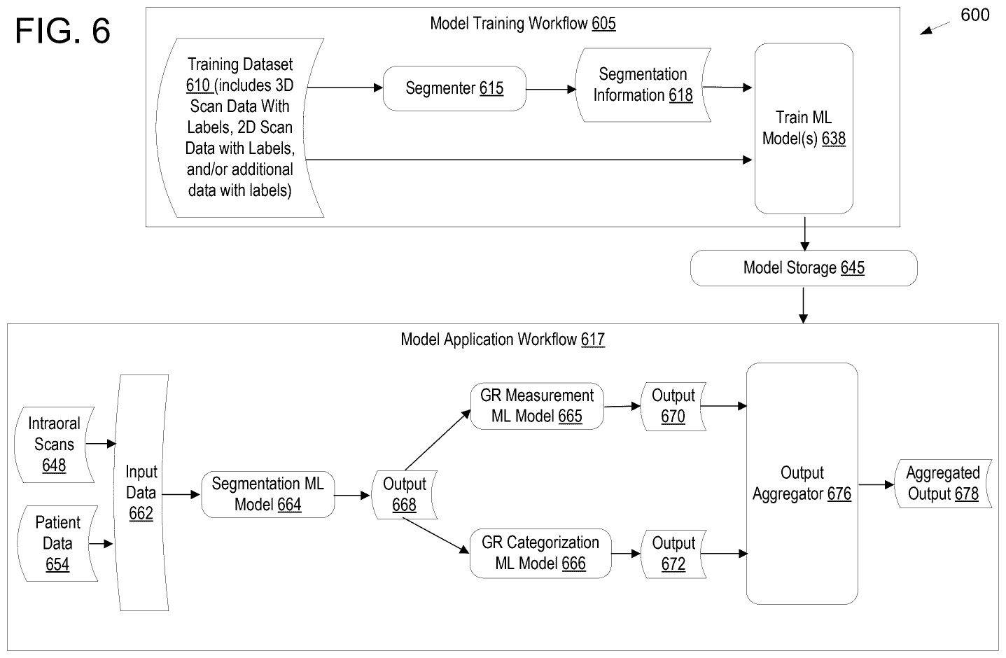

In some embodiments, image segmentation engine 213 can segment scan data (e.g., 2D images, intraoral scans, 3D models, etc.) into features, such as individual teeth (including tooth number), gingiva, cementum, enamel, and/or CEJ. In some embodiments, the image segmentation engine 213 can receive scan data 251 of a patient's dentition. In some embodiments, the input preprocessing engine 212 can convert the image scan data 251 into a 3D model, e.g., using sparse voxel segmentation, mesh segmentation, or point-based segmentation. The image segmentation engine 213 can include a trained machine learning model that takes scan data (e.g., 2D images, intraoral scans, 3D models, etc.) as input, and outputs segmentation data indicating the dental features (tooth number, gingiva, cementum, enamel, and/or CEJ). In some embodiments, image segmentation engine 213 can correspond to segmenter 415 and/or segmentation ML model 664 of FIG. 6, and is further described with respect to, FIG. 6. Generated segmentation information may be stored as segmentation data in segmentation data 254 in embodiments.

In some embodiments, image segmentation engine 213 is or includes a trained machine learning model that has been trained to perform semantic segmentation and/or instance segmentation of oral structures (e.g., to determine sizes, shapes, locations, etc. of individual teeth, gingiva, cementum, CEJ, etc.). Applicant hereby incorporates by reference the following application as if set forth fully here, as an example of a machine learning dental segmentation system and method, and training of such a machine learning segmentation system: U.S. patent application Ser. No. 17/138,824.

In some embodiments, the input preprocessing engine 212 can project 3D scan data 251 (e.g., a 3D model of a dental arch or intraoral scan) into 2D, e.g., using a mesh projection algorithm. The image segmentation engine 213 can then segment the 2D scan data using 2D segmentation techniques. The resulting segmentation can then be back-projected onto the 3D model or intraoral scan, and stored in segmentation data 254. Applicant hereby incorporates by reference the following application as if set forth fully here, as an example 2D tooth segmentation: U.S. patent application Ser. No. 18/446,445.

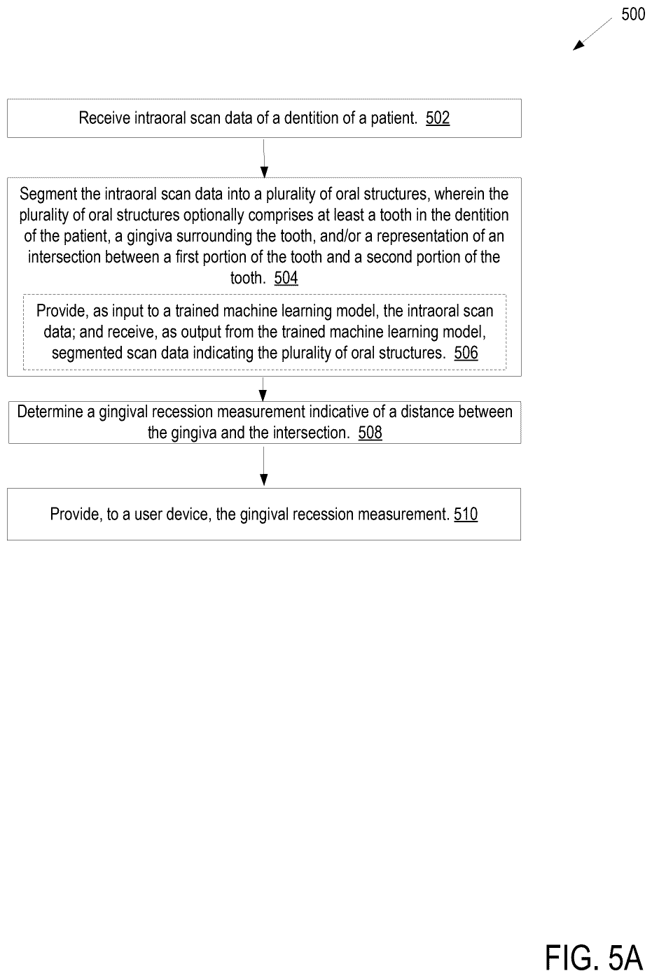

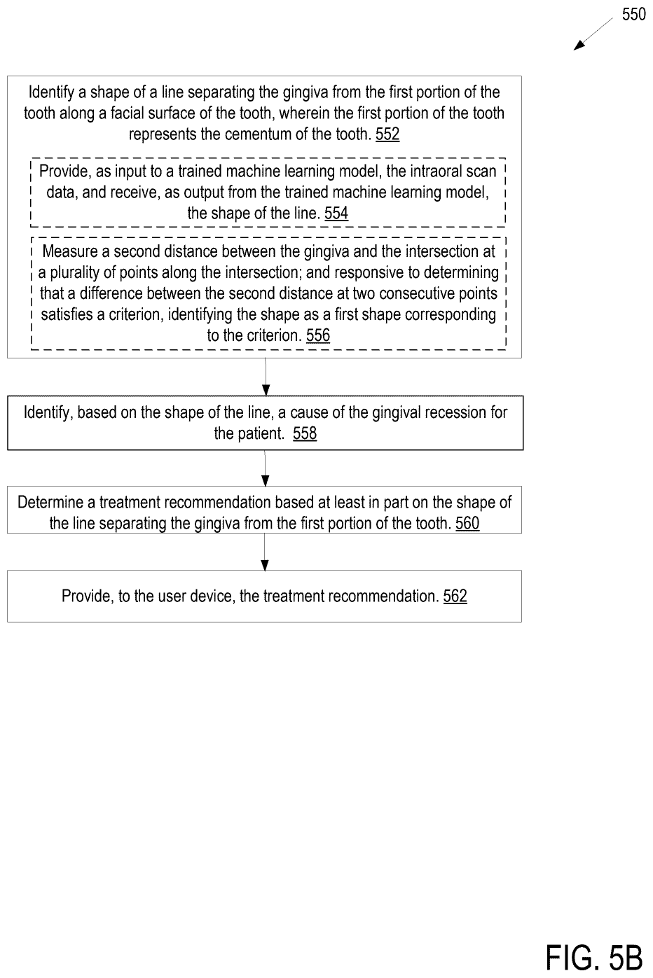

In some embodiments, the gingival recession measurement and categorization engine 220 can be a software program hosted by a device (e.g., computing device 105) to determine gingival recession measurement and/or categorization of intraoral scan data. In some embodiments, the gingival recession measurement and categorization engine 220 can measure and/or categorize the gingival recession of a patient's dentition represented by segmentation data 254. The gingival recession measurement and categorization engine 220 can include a measurement module and a categorization module. In some embodiments, the measurement module can implement a trained machine learning model that takes as input the segmentation data 254 of a patient, and provides as output the measurement of the gingival recession for each tooth in the patient's dentition. The segmentation data 254 may include instance segmentation data, and may indicate a tooth number for each tooth on the patient's dental arches which has been identified and segmented. In some embodiments, the measurement module can implement a measurement algorithm that determines the gingival recession measurements from segmentation data 254, without using a trained machine learning model. The gingival recession measurement techniques are further described with respect to FIG. 5A and FIG. 6. The measured gingival recession (GR) can be included in GR measurement data 253 and stored in recession measurement and categorization data store 144 in embodiments.

In some embodiments, the gingival recession measurement and categorization engine 220 can implement a trained machine learning model that receives, as input, the segmentation data 254 of a patient and provides as output the categorization of the gingival recession for each tooth in the patient's dentition. In some embodiments, the gingival recession measurement and categorization engine 220 can implement a recession-type identification algorithm to categorize the recession. In some embodiments, the gingival recession measurement and categorization engine 220 can implement a categorization algorithm that determines the gingival recession category from segmentation data 254, without using a trained machine learning model. In some embodiments, the gingival recession can be categorized as a “U” shape or a “V” shape for each tooth for which gingival recession is detected. That is, the shape of the gingival line along the facial surface of a tooth can resemble a “U” or a “V.” The gingival recession measurement and categorization engine 220 can classify the gingival recession as either “U” or “V” shaped. The gingival recession measurement techniques are further described with respect to FIG. 5B and FIG. 6. The categorizations of the gingival recessions can be included in GR categorization data 255.

In some embodiments, the treatment recommendation engine 225 can be a software program hosted by a device (e.g., computing device 105) to determine a treatment recommendation for gingival recession of a patient. In some embodiments, the treatment recommendation engine 225 can access the GR measurement data 253, the GR categorization data 255, patient data 256, and/or occlusion data 257 to determine a treatment recommendation for a patient. In some embodiments, treatment recommendation engine 225 is a rules-based engine that includes rules that relate various combinations of different GR measurement data 253, the GR categorization data 255, patient data 256, and/or occlusion data 257 to different treatment recommendations. In some embodiments, treatment recommendation engine 225 includes one or more trained machine learning models that have been trained to receive as an input GR measurement data 253, the GR categorization data 255, patient data 256, and/or occlusion data 257, and to output treatment recommendations.

A “U” shaped gingival recession line can be indicative of poor dental hygiene. The treatment recommendation engine 225 can identify a treatment recommendation from treatment recommendation rules of recommendation data store 242 that corresponds to “U” shaped gingival recession. A “V” shaped gingival recession line may indicate malocclusion of the teeth. The treatment recommendation engine 225 can analyze the 3D geometric surface of scan data 251 to identify an abfraction of the tooth that has a “V” shaped gingival recession line. An abfraction includes tooth damage or wear along the cervical margin due to mechanical forces, and not caused by decay. The treatment recommendation engine 225 can analyze the 3D geometric surface of the tooth near the occlusal and/or incisal tooth wear (e.g., of scan data 251) to detect any potential signs of occlusal trauma (e.g., a chip, a crack, a fracture, and/or wear of the tooth, presenting as a flattened surface or exposed dentin). The treatment recommendation engine 225 can analyze the 3D geometric surface of the tooth (e.g., of scan data 251) to identify potential restoration, and may identify wear of the restoration in some embodiments. The combination of the “V” shaped gingival recession line, along with an identification of an abfraction, occlusal trauma, tooth wear, and/or another detected abnormality, can indicate an area of excessive tooth collision. In some embodiments, the treatment recommendation engine 225 can use the occlusion data 257 of the patient's dentition to identify area(s) of heavy collision. The treatment recommendation engine 225 can identify a treatment recommendation from recommendation data store 242 corresponding to a “V” shaped gingival recession line. In some embodiments, the treatment recommendation engine 225 can recommend orthodontic treatment (e.g., aligners) to treat the malocclusions causing the gingival recession. In some embodiments, the treatment recommendation engine 225 can recommend modifications to an existing orthodontic treatment plan to prevent further progression of the gingival recession. In some embodiments, the treatment recommendation engine 225 can use a trained machine learning model to generate a treatment recommendation plan (e.g., an orthodontic treatment plan) for the patient, and can store the generated treatment recommendation plan in recommendation data store 242.

In some embodiments, the report generation engine 230 can be a software program hosted by a device (e.g., computing device 105) to generate a gingival recession and/or treatment report for one or more patients. In some embodiments, the report generation engine 230 can automatically generate a report, which can be shared with and/or presented to a patient, e.g., on computing device 160. In some embodiments, the report generation engine 230 can generate a report that summarizes the gingival recession measurements and/or categorizations, and the treatment recommendation(s) for a particular patient. The report can include the recession measurements and/or categorizations over time, including the longitudinal progression of the recession(s). The report generation engine 230 can access the treatment recommendation(s) from recommendation data store 242, and/or the GR measurement data 253, GR categorization data 255, and/or the patient data 256 from recession measurement and categorization data store 144. The report generation engine 230 can provide the generated report, which can include the GR measurement data 253 and/or the treatment recommendation(s), to a user device (e.g., computing device 160). In some embodiments, the report can be presented to the user, and can include the intraoral scan, the 3D model of the patient's dental arch, 2D images of the patient's dental arch, etc., showing abfractions and the gingival recession, along with the patient's occlusogram showing the areas of heavy collision. In some embodiments, the report can include radiographs with the AI-detected areas of vertical bone loss for teeth with heavy occlusion.

In some embodiments, computing device(s) 160 can be a user device. In some embodiments, the user device 160 can be used by a dental professional (e.g., a doctor, a dentist, a hygienist, and/or a technician) to educate a patient regarding the patient's dental health. In some embodiments, the user device 160 can be used by a patient to review their dental health. The user device 160 can include a user interface (UI) to display the generated report, the GR measurement data, the treatment recommendation(s) stored in recommendation data store 142, patient data 256, occlusion data 257, and/or the scan images of scan data 251 optionally overlaid with the segmentation data 254, occlusion data 257, and/or the gingival recession measurements and/or categorizations.

FIG. 3 illustrates a block diagram of an example system 300 for detecting and/or assessing TMD, in accordance with some embodiments of the present disclosure. System 300 includes a computing device 105 that may be coupled to one or more computing devices 160, oral state capture system(s) 110, and/or a data store 108. In some embodiments, computing device 160, oral state capture system 110, computing device 105, and/or data store 108 of FIG. 3 can perform the same function as computing device 160, oral state capture system 110, computing device 105, and/or data store 108 of FIG. 1.

Computing devices 105 and/or 160 may each include a processing device, memory, secondary storage, one or more input devices (e.g., such as a keyboard, mouse, tablet, and so on), one or more output devices (e.g., a display, a printer, etc.), and/or other hardware components. Computing device 105 may be connected to a data store 108 either directly or via a network (e.g., network 150). The network 150 may be a local area network (LAN), a public wide area network (WAN) (e.g., the Internet), a private WAN (e.g., an intranet), or a combination thereof. The computing device 105 may additionally or alternatively be connected to computing device(s) 160 and/or oral state capture systems 110 via a network 150, which may be a local area network (LAN), a public wide area network (WAN) (e.g., the Internet), a private WAN (e.g., an intranet), or a combination thereof. In some embodiments, oral state capture system(s) 110 connect to computing device(s) 105 directly via a wired or wireless connection.

Data store 108 may be an internal data store, or an external data store that is connected to computing device 105 directly or via a network. Examples of network data stores include a storage area network (SAN), a network attached storage (NAS), and a storage service provided by a cloud computing service provider. Data store 108 may include a file system, a database, or other data storage arrangement.

In some embodiments, data store 108 can include a recommendation data store 142 and/or a TMD diagnostics data store 144. In some embodiments, the recommendation data store 142 can include treatment recommendation rules, e.g., used by treatment recommendation engine 325 to identify treatment options. In some embodiments, the recommendation data store 142 can include the treatment recommendations and/or reports generated by treatment recommendation engine 325 and/or report generation engine 330. The TMD diagnostics data store 144 can include scan data 351 (e.g., CBCT scan data), audio data 352, video data 353, segmentation data 354, classification data 355, and/or patient data 356. In some embodiments, scan data 351, audio data 352, video data 353, segmentation data 354, classification data 355, and/or patient data 356 can reference a patient identifier.

Patient data 356 can include a patient chart (e.g., patient dental chart), which can include answers that the patient provided to a questionnaire. The questionnaire may be presented to the user in a clinical setting, e.g., by a clinician, technician, or medical professional. In some embodiments, the questionnaire may have been presented to the patient on a user device of the patient (e.g., computing device 160). In some embodiments, patient data 356 can include a history of the patient's TMD diagnostics data. The patient's history may include, for example, intraoral scans, 2D images and/or 3D models of the patient's dental arch(es) generated at various points in time, audio and/or video recordings of the patient jaw movements, prior diagnoses of TMD, and/or prior assessments of other medical ailments.

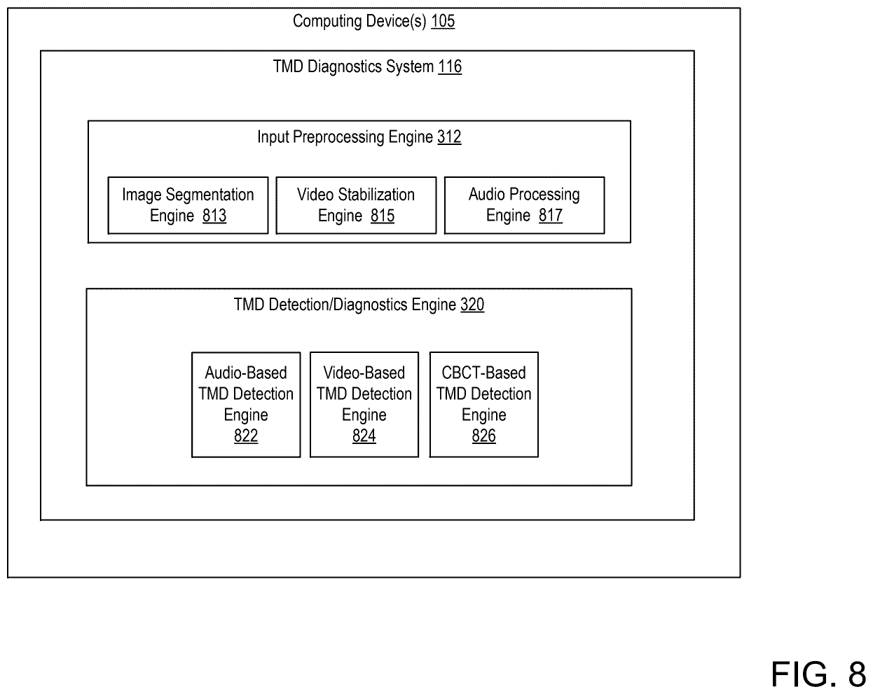

In some embodiments, scan data 351 can include scan data generated by a CBCT machine (e.g., a CBCT scanner 163). A CBCT machine is a type of x-ray machine that uses a cone-shaped x-ray beam to capture data about the patient's anatomy. The CBCT scan can generate multiple (e.g., 150-200) images from a variety of angles. In some embodiments, the data captured can be used to reconstruct a 3D image of the patient's teeth, mouth, jaw, neck, ear, nose, and/or throat. The scan data 351 can be captured with the patient's jaw in an open-position (e.g., using rubber blocks to keep the jaw in position during the scanning process), and/or in a closed-position. The scan data 351 can include indicators of TMD, such as abnormal size, shape, location of the joint bones of the TMJ (e.g., condylar head, fossa/articular eminence, position of the condyle to the articular fossa, etc.). Examples of TMJ irregularities that may be indicative of TMD are further described with respect to FIGS. 8-10.

In some embodiments, audio data 352 can include an audio recording of a patient's jaw movements. In some embodiments, the audio data 352 can be captured by a microphone 161, camera 162, intraoral scanner 164, and/or ECI device 166. The audio data 352 can be captured as the patient opens, closes, laterally moves, and/or protrusively moves of the jaw. The audio data 352 can include one or more sound indicators of TMD, such as a clicking sound, a popping sound, a snapping sound, crepitus, and/or any other sound indicative of TMD.

In some embodiments, video data 353 can include a video recording of a patient's jaw movements. In some embodiments, the video data 353 can be captured by a camera 162. The video data 353 can be captured as the patient opens, closes, laterally moves, and/or protrusively moves of the jaw. The video data 353 can include one or more visual motion-based indicators of TMD, such as catching and/or popping during jaw movement.

In some embodiments, segmentation data 354 can include the segmented image data, as generated by input preprocessing engine 312. The segmentation data 354 can include segmented scan data 351 and/or segmented video data 353 (e.g., segmentation of frames of video data 353). The segmentation process is further described with respect to FIG. 8.

In some embodiments, classification data 355 can include rules for classifying TMD. In some embodiments, classification data 355 can include classification(s) of a patient's TMD, as generated by TMD detection/diagnostics engine 320.

In some embodiments, oral state capture system(s) 110 can include a microphone 161; a camera 162 (e.g., a video camera); a CBCT scanner 163 (and/or another imaging device, such as a CT scanner); an electronic compliance indicator (ECI) device 166 or other dental appliance to be worn by a patient that includes a microphone; an intraoral scanner 164; and/or optionally a computing device 165. The oral state capture system 110 can obtain audio, video, and/or image-based scans of a patient's dentition, jaw, and/or jaw movements. In some embodiments, the microphone 161, camera 162, CBCT scanner 163, intraoral scanner 164, the ECI device 166, and/or processing device 165 can be combined. For example, in some embodiments, the microphone can be built into the base of a scan wand of the intraoral scanner 164, which can be used to capture audio while a technician is using the intraoral scanner 164 for other diagnostic capabilities (e.g., to perform intraoral scanning). In some embodiments, oral state capture system 110 includes a dental appliance such as an aligner, palatal expander, etc. that includes a microphone. As another example, in some embodiments, the processing device 165 can be part of the intraoral scanner 164, CBCT scanner 163, and/or ECI device 166. In some embodiments, processing device 165 can be part of computing device 160, computing device 105, and/or a separate device (not shown), and the oral state capture system 110 can send captured data (e.g., scan data, audio data, and/or video data) for processing on a separate device. In one embodiment, oral state capture system 110 includes a patient or client device that can take 2D or 3D images, videos, and/or audio recordings of the patient's oral cavity in a non-clinical setting (e.g., at a patient's home).

In some embodiments, oral state capture system 110 may include a scanning system (e.g., CBCT scanner 163, intraoral scanner 164, and/or ECI device 166) that can perform scanning of the patient's mouth, jaw, head, oral cavity, and/or other area of the patient where the patient may be experiencing TMD-related symptoms. The scanning may be performed to generate a plurality of scans of the patient's jaw movements, which may be combined to generate a three dimensional (3D) model of a dentition and/or jaw of a patient. In some embodiments, oral state capture system 110 may include an imaging device, which may be a 2D or 3D imaging device, such as a digital camera, mobile phone, tablet computer, and so on. In some embodiments, the oral state capture system 110 may include a CBCT scanner 163, to capture CBCT scans of the patient's jaw. A CBCT scanner 163 is a type of x-ray machine that uses a cone-shaped x-ray beam to capture data about the patient's anatomy. The CBCT scanner 173 can generate multiple (e.g., 150-200) images from a variety of angles.

In some embodiments, microphone 161 capture sounds of a patient's jaw movements. The microphone 161 can convert sound into audio signals. In some embodiments, microphone 161 can be an external microphone that is connected to an analog to digital converter (ADC) and a digital capture component. In some embodiments, the microphone 161 can be integrated into a device with additional components, such as the ADC, digital capture, and/or the processing device 165. For example, microphone 161 can be part of a patient's mobile device (e.g., smart phone). In some embodiments, the microphone 161 can be a custom device, such as stethoscope microphone that may be optimized for listening and optionally recording sounds from the human body. In some embodiments, the microphone 161 can be attached to or built into the intraoral scanner 164.

In some embodiments, the microphone 161 of oral state capture system 110 (e.g., either a standalone microphone or a microphone that is part of another device) may be a bone conduction microphone that picks up sound vibrations from the user's jawbone, rather than from the air. The bone conduction microphone can be placed against the patient's jawbone, and can record the sound vibrations as the patient opens and/or closes their mouth. The bone conduction microphone can detect and convert the vibrations into electrical signals, and a processing device and convert the signals to digital signals. In some embodiments, the microphone 161 can be connected to a processing device 165 via Bluetooth®.

In some embodiments, the camera 162 can include a video camera and optionally, a video capture system. In some embodiments, the camera 162 can be a standalone external camera. In some embodiments, the camera 162 can be integrated into a device, such as a patient's mobile device (e.g., smart phone), or attached to an intraoral scanner 164. In some embodiments, processing device 165 is integrated in the camera 162 device. The video capture system may include an application that can extract video and/or audio captured by the camera 162. The video capture system may store the extracted video data in video data 353, and the extracted audio data in audio data 352. The video capture system may capture 2D or 3D videos in embodiments.

In one embodiment, oral state capture system 110 includes an intraoral scanning system comprising a scanner 164 for obtaining intraoral scans (e.g., 3D data) of a patient's dentition and optionally a computing device 165. Alternatively, oral state capture system 110 may include an intraoral scanner 164, and computing device 105 may connect to the intraoral scanner 164 to effectuate intraoral scanning. In embodiments, computing device 105 or another computing device of oral state capture system 110 includes an intraoral scan application that processes intraoral scans generated by the intraoral scanner to generate 3D models of the patient's upper and/or lower dental arches.

Intraoral scanner 164 may include a probe (e.g., a hand held probe) for optically capturing three-dimensional structures, and a microphone (e.g., microphone 161). The intraoral scanner may be used to perform an intraoral scan of a patient's oral cavity. An intraoral scan application running on computing device 105 (or on another computing device of oral state capture system 110) may communicate with the scanner to effectuate the intraoral scan. A result of the intraoral scan may be scan data 351 that may include one or more sets of intraoral scans, which may include intraoral images. Each intraoral scan may include a two-dimensional (2D) or 3D image that may include depth information (e.g., a height map) of a portion of a dental site. In embodiments, intraoral scans include x, y and z information. In one embodiment, the intraoral scanner generates numerous discrete (i.e., individual) intraoral scans. In some embodiments, the intraoral scan application may extract the audio recorded by the microphone attached to, or built in to, the intraoral scanner 164. The extracted audio recording can be stored in audio data 352. In some embodiments, the audio recorded using an intraoral scanner 164 may sounds indicative of TMD that vary (e.g., are of a different frequency) from audio recorded from outside of the oral cavity. Thus, audio data 352 may include an indication of whether the stored audio data is recorded intraorally or outside of the oral cavity.

In some embodiments, the oral state capture system 110 can include an ECI device 166. In some embodiments, the ECI device 166 can be used to accurately monitor of a patient's compliance to a prescribed aligner schedule. For instance, an aligner that is ECI-capable can have one or more sensors designed to detect temperature and/or proximity to a patient's tooth. The sensors can pair to a mobile phone, e.g., via a Bluetooth-enabled “smart” aligner case, and can receive and/or transmit data between the mobile phone and the ECI. In some embodiments, the ECI device 166 can capture sound, and the processing device 165 can store the captured sound in audio data 352. In some embodiments, data generated from the ECI device 166 can be used to infer movement(s) of the jaw, which can be used to identify an indicator of the TMD. In some embodiments, the ECI device 166 can include a pressure sensor that can measure pressure and can convert the measured physical pressure exerted on it into an electrical signal. The pressure sensor on the occlusal surface of the teeth can detect the occlusal force or biting pressure, which can be used to detect bruxism (grinding and/or clenching of the teeth). The pressure sensor can include a sensing element that directly responds to pressure, a transducer that converts the physical change in the sensing element into an electrical signal, a signal conditioning component that can amplify, filter, and/or convert the signal into a digital signal, and/or an output component that can transmit the conditioned signal to a processing device. For example, the pressure sensor can be used to measure and analyze the forces exerted during various dental procedures and treatments, such as occlusal analysis, implantology, orthodontics, prosthodontics, and/or periodontology. In some embodiments, the pressure sensor can measure electrical activity recorded during execution of a sequence of actions (e.g., bruxism-related events such as teeth clenching and teeth grinding, etc., and/or bruxism-unrelated events such as swallowing, lightly nodding the head, lightly shaking the head, speaking, etc.). In some embodiments, the pressure sensor can record a time-averaged value during execution of a particular sequence of actions. The pressure sensor can detect, record, and/or transmit signals to the processing device 165. The pressure data (e.g., the detected signals) can indicate clenching or grinding of a patient. In some embodiments, the pressure sensor can be attached to a processing device in oral state capture system 110, or can be otherwise connected to a processing device in oral state capture system 110.

In some embodiments, oral state capture system 110 is connect to data store(s) 108 either directly or via network 150. In some embodiments, oral state capture system 110 transmits image data (e.g., CBCT scan data), audio recording data, and/or video recording data to data store 108 for storage therein.

In some embodiments, computing device 105 is a desktop computer, a laptop computer, a server computer, etc., located at a doctor office. In some embodiments, computing device 105 is a server computing device (e.g., of a data center) that may be accessed from client devices (e.g., client devices of doctors, patients, etc.). In some embodiments, computing device 105 is a virtual machine. For example, computing device 105 may be a virtual machine that runs in a cloud computing environment.

In some embodiments, computing device 105 includes a TMD diagnostics system 116, which may include an input preprocessing engine 312, a TMD detection/diagnostics engine 320, a treatment recommendation engine 325, and/or a report generation engine 330. TMD diagnostics system 116 may include software, hardware and/or firmware configured to perform one or more operations with respect to detecting, assessing, diagnosing, and/or treating TMD and, optionally, other dental conditions related to TMD.

In some embodiments, input preprocessing engine 312 can be a software program hosted by a device (e.g., computing device 105) to process scan data 351, audio data 352, and/or video data 353. Input preprocessing engine 312 may perform one or more operations on scan data 351, audio data 352, and/or video data 353 (e.g., on one or more frames of a video in video data 353) to prepare the scan data 351, audio data 352, and/or video data 353 for analysis of TMD. Input preprocessing engine 312 may perform operations such as filtering, stabilizing, cropping, image enhancement (e.g., to sharpen an image), segmentation, and/or other operations. In some embodiments, if scan data 351 does not include a 3D model of a dental arch (e.g., includes 2D images or intraoral scans but no 3D models of dental arches), input preprocessing engine 312 may process the 2D images and/or intraoral scans to generate one or more 3D models (e.g., as discussed above). In some embodiments, 3D models may be generated from 2D images (e.g., such as those taken by a patient device such as a patient's mobile phone). In some embodiments, the input preprocessing engine 312 can project 3D scan data 351 (e.g., a 3D model of a dental arch or intraoral scan) into 2D, e.g., using a mesh projection algorithm. The image segmentation engine 313 can then segment the 2D scan data using 2D segmentation techniques. The resulting segmentation can then be back-projected onto the 3D model or intraoral scan, and stored in segmentation data 354. Applicant hereby incorporates by reference the following application as if set forth fully here, as an example 2D tooth segmentation: U.S. patent application Ser. No. 18/446,445.

The input preprocessing engine 312 is further described with respect to FIG. 8.

In some embodiments, the TMD detection/diagnostics engine 320 can be a software program hosted by a device (e.g., computing device 105) to detect, assess, and or diagnose TMD in a patient. In some embodiments, the TMD detection/diagnostics engine 320 can detect, assess, and/or diagnose TMD of a patient represented by scan data 351, audio data 352, video data 353, and/or segmentation data 354. The TMD detection/diagnostics engine 320 can analyze the scan data 351, audio data 352, video data 353, and/or segmentation data 354 to identify an indicator of the TMD. The TMD detection/diagnostics engine 320 can classify scan data 351, audio data 352, video data 353, and/or segmentation data 354 to indicate the a likelihood of the presence of TMD, and can store the classification(s) in classification data 355. The TMD detection/diagnostics engine 320 is further described with respect to FIG. 8.

In some embodiments, the treatment recommendation engine 325 can be a software program hosted by a device (e.g., computing device 105) to determine a treatment recommendation for TMD. In some embodiments, the treatment recommendation engine 325 can access the scan data 351, audio data 352, video data 353, segmentation data 354, classification data 355, and/or patient data 356 to determine a treatment recommendation for a patient. In some embodiments, treatment recommendation engine 325 is a rules-based engine that includes rules that relate various combinations of different scan data 351, audio data 352, video data 353, segmentation data 354, classification data 355, and/or patient data 356 to different treatment recommendations. In some embodiments, treatment recommendation engine 325 includes one or more trained machine learning models that have been trained to receive as an scan data 351, audio data 352, video data 353, segmentation data 354, classification data 355, and/or patient data 356, and to output treatment recommendations.

Applicant hereby incorporates by reference the following application as if set forth fully herein, as an example of an ortho-restorative treatment planning system and method for treating or preventing TMD: US. Pat. Pub. No. 20230414323A1.

Applicant hereby incorporates by reference the following application as if set forth fully herein, as an example of a method and system for the treatment of temporomandibular joint dysfunction with aligner therapy: US. Pat. Pub. No. 20240099816A1.

In some embodiments, the report generation engine 330 can be a software program hosted by a device (e.g., computing device 105) to generate a TMD detection and/or treatment report for one or more patients. In some embodiments, the report generation engine 330 can automatically generate a report, which can be shared with and/or presented to a patient, e.g., on computing device 160. In some embodiments, the report generation engine 330 can generate a report that summarizes the TMD detection, assessment, and/or diagnosis, and the treatment recommendation(s) for a particular patient (e.g., as determined by treatment recommendation engine 325). The report can include the TMD symptoms and detected indicators over time. The report generation engine 330 can access the treatment recommendation(s) from recommendation data store 342, and/or scan data 351, audio data 352, video data 353, segmentation data 354, classification data 355, and/or patient data 356 from recession measurement and categorization data store 344. The report generation engine 330 can provide the generated report to a user device (e.g., computing device 160).

In some embodiments, computing device(s) 160 can be or include a user device. In some embodiments, the user device 160 can be used by a dental professional (e.g., a doctor, a dentist, a hygienist, and/or a technician) to educate a patient regarding the patient's dental health. In some embodiments, the user device 160 can be used by a patient to review their dental health. The user device 160 can include a user interface (UI) to display the generated report, the treatment recommendation(s) stored in recommendation data store 142, patient data 356, and/or the images of scan data 351 optionally overlaid with the segmentation data 354, classification data 355, and/or the detected TMD indicators.

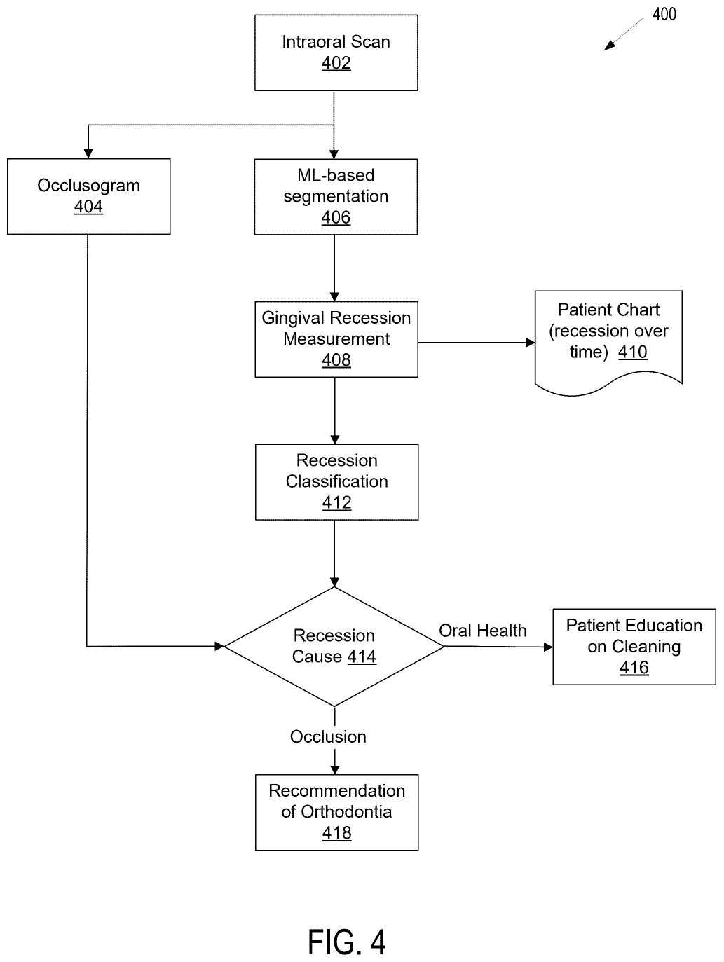

FIG. 4 illustrates a flow diagram of a method 400 for measuring and categorizing gingival recession of a patient, in accordance with some embodiments of the present disclosure. Method 400 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, method 400 may be performed by the processing devices and the associated algorithms, e.g., as described in conjunction with FIGS. 1, 2. In embodiments, method 400 is performed by processing logic comprising hardware, software, firmware, or a combination thereof. In certain embodiments, method 400 may be performed by a single processing thread. Alternatively, method 400 may be performed by two or more processing threads, each thread executing one or more individual functions, routines, subroutines, or operations of the method. In an illustrative example, the processing threads implementing method 400 may be synchronized (e.g., using semaphores, critical sections, and/or other thread synchronization mechanisms). Alternatively, the processing threads implementing method 400 may be executed asynchronously with respect to each other. Therefore, while FIG. 4 and the associated descriptions list the operations of method 400 in a certain order, in some embodiments, at least some of the described operations may be performed in parallel and/or in a different order. In some embodiments one or more operations of method 400 is not performed.

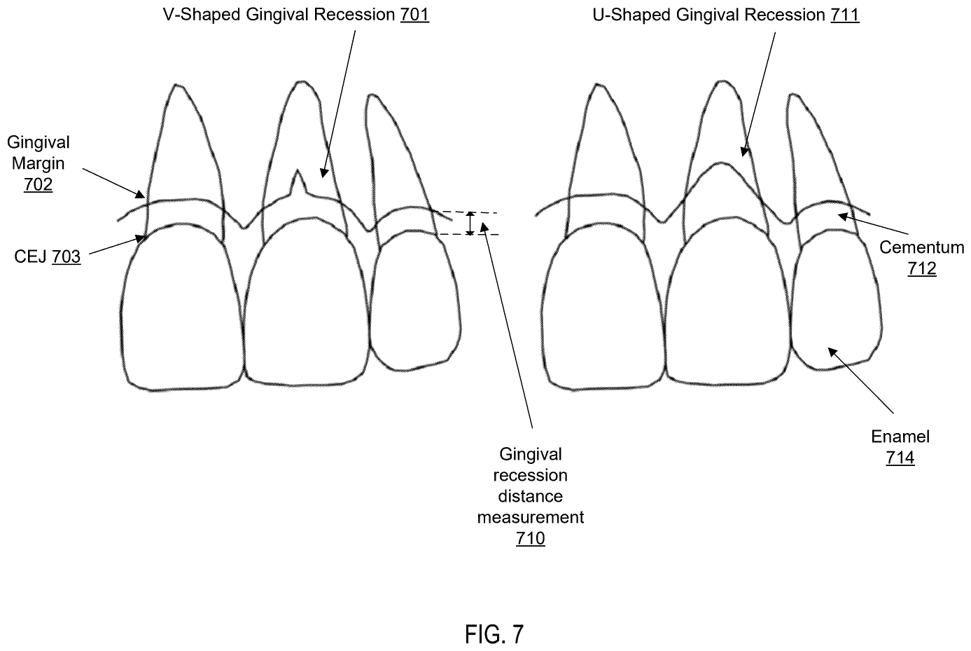

At block 402, processing logic can receive intraoral scan data of a patient's dentition. The intraoral scan data may include one or more intraoral scans of a dental site, one or more color 2D images of the dental site, one or more 3D models of the dental site, etc. At block 404, an occlusogram can be generated from the intraoral scan data. At block 406, the intraoral scan data is segmented into individual numbered teeth, gingiva, and the CEJ (for one or more teeth). In some embodiments, the scan data is segmented into individual numbered teeth, the gingiva, the cementum of one or more teeth, and optionally the enamel of one or more teeth. Processing logic can then identify the CEJ for a tooth as the intersection between the cementum and the enamel of the tooth. An example CEJ is illustrated in FIG. 7.

The ML-based segmentation at block 406 can be performed from an intraoral scan and/or 3D model of a dental site in 3D, e.g., using sparse voxel segmentation, mesh segmentation, or point-based segmentation. In some embodiments, the intraoral scan and/or 3D model can be projected into 2D using one of a variety of mesh projection algorithms. The 2D projection (e.g., 2D image) can then be segmented, at block 406, using 2D segmentation techniques, such as a neural network for 2D segmentation examples of which include U-Net, MANet, nn-Unet, etc. The resulting segmentation information can then be back-projected from 2D onto the 3D model or intraoral scan.

At block 408, processing logic can identify, on the segmented intraoral scan data (e.g., segmented intraoral scan, segmented 3D dental arch model, etc.), the gingival line (dividing the tooth from the gingiva) for each tooth. For teeth with gingival recession, processing logic can also identify the CEJ (either as a segmented line/region, or as the boundary between the cementum and enamel). The gingival line and/or CEJ may be identified using traditional image processing techniques and/or machine learning techniques in embodiments.

For teeth with a visible CEJ (e.g., for teeth with gingival recession), the measurement algorithm of block 408 can identify the maximum distance between the gingiva and the CEJ along the facial surface of the tooth. In one embodiment, the maximum distance between the gingiva and CEJ for a tooth may be determined by determining, for one or more points on the CEJ, a distance to a closest point on the gingival line. The point on the CEJ of the tooth having the largest distance may then be selected as the maximum distance. In one embodiment, a tooth long axis (TLA) is determined for a tooth. The maximum distance between the gingiva and CEJ for a tooth may be determined by determining, for one or more points on the CEJ, a distance along the TLA for the tooth to the gingival line. The point on the CEJ of the tooth having the largest distance may then be selected as the maximum distance. This maximum distance between the gingiva and the CEJ for a tooth indicates the amount of recession on that particular tooth in embodiments. At block 410, processing logic records the measurement in the patient's chart. The measurement can be recorded automatically, or manually by a technician. The patient's chart has a record of the gingiva recession measurements over time. The techniques described herein result in the longitudinal data stored in the patient's chart to be a more accurate measurement of recession over time than recession measurements performed manually (e.g., by different dentists, hygienists, and/or technicians).

At block 412, process logic classifies (or categorizes) the type of the recession. Processing logic can use the gingival margin and the CEJ to categorize the type of the recession in embodiments. The recession can be categorized as a “U” shape or a “V” shape. The shape of the recession is further described with respect to FIG. 7. At block 412, recession classification can be performed by a machine learning classification algorithm (e.g., a trained machine learning model such as a convolutional neural network) or a geometric assessment of the various distances between the CEJ and the gingival margin along the tooth surface. Using either ML or geometric assessment, processing logic can analyze the geometric shape formed by the gingival line, and categorize the recession as either “U” or “V” shaped.

Processing logic can identify a potential cause of the measured gingival recession, and optionally, can recommend a treatment in embodiments. At block 414, processing logic can identify a potential cause of the gingival recession. A “U” shaped recession is most often associated with an oral hygiene issue. A “V” shaped recession can be associated with misalignment of the teeth. In embodiments, processing logic receives and processes both the recession classification information generated at block 412 and the occlusogram 404 generated at block 404 to assess recession cause. An abfraction includes tooth damage or wear along the cervical margin due to mechanical forces, and may not be caused by decay. If the recession is “V” shaped and the tooth has an abfraction, the occlusogram is likely to show heavy collisions. These collisions can be the cause of the recession, and an orthodontic treatment (e.g., dental aligners) may correct the malocclusion and support the patient's dental health. In some embodiments, processing logic can analyze the 3D geometric surface of the tooth near the CEJ, and can identify one or more abfractions. In some embodiments, processing logic can analyze the 3D geometric surface of the tooth near the occlusal/incisal to detect any potential signs of occlusal trauma (e.g., a chip, a crack, a fracture, and/or wear of the tooth, e.g., displayed as a flattened surface or exposed dentin). In one embodiment, such 3D geometric analysis is performed using one or more geometric assessment techniques. In one embodiment, such 3D geometric analysis is performed using a trained machine learning model trained to identify abfractions.

If at block 414 a recession cause is determined to be related to oral health then the method proceeds to block 416. If at block 414 a recession cause is determined to be related to patient occlusion, then the method proceeds to block 418. At block 416, processing logic can provide patient education on cleaning (e.g., how to better care for their teeth). At block 418, processing logic can provide a recommendation of orthodontia, including, e.g., a recommendation to modify a current orthodontic treatment plan to prevent further progression of the recession.

In some embodiments, the information generated from method 400 can be presented to a patient. In some embodiments, the information generated from method 400 is presented as an overlay displayed over the intraoral scan data (e.g., over a 3D model of the patient's dental arches). The overlays may include visualizations showing abfractions and/or gingival recession, visualizations showing tooth collusions (e.g., from the occlosugram) and/or other information in order to educate the patient on the need for orthodontia for their dental health. In some embodiments, the information generated from method 400 is presented as an overlay displayed over a radiograph of the patient's teeth, showing the detected areas of vertical bone loss (often associated with V-shaped gingival recession due to occlusal trauma) and the visualizations showing abfractions and/or other information to educate the patient.

FIG. 5A illustrates a flow diagram of a method 500 for measuring gingival recession, in accordance with some embodiments of the present disclosure. FIG. 5B illustrates a flow diagram of a method 550 for categorizing gingival recession, in accordance with some embodiments of the present disclosure. Methods 500, 550 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, methods 500, 550 may be performed by the processing devices and the associated algorithms, e.g., as described in conjunction with FIGS. 1, 2. In certain embodiments, methods 500, 550 may be performed by a single processing thread. Alternatively, methods 500, 550 may be performed by two or more processing threads, each thread executing one or more individual functions, routines, subroutines, or operations of the method. In an illustrative example, the processing threads implementing methods 500, 550 may be synchronized (e.g., using semaphores, critical sections, and/or other thread synchronization mechanisms). Alternatively, the processing threads implementing methods 500, 550 may be executed asynchronously with respect to each other. Therefore, while FIGS. 5A-5B and the associated descriptions list the operations of methods 500, 550 in a certain order, in some embodiments, at least some of the described operations may be performed in parallel and/or in a different order. In some embodiments one or more operations of methods 500, 550 is not performed.

At block 502, processing logic receives intraoral scan data of a dentition of a patient. For example, processing logic can receive scan data 251 generated by oral state capture system 110 of FIGS. 1, 2. For example, the intraoral scan data can include one or more intraoral scans generated by an intraoral scanner. The intraoral scan data can include a 3D model of the dentition of the patient generated from multiple intraoral scans. In some embodiments, the scan data of the dentition of the patient can be 3D scan data that includes color information.

In some embodiments, the intraoral scan data includes three-dimensional scan data, two-dimensional near infrared scan data (e.g., 2D NIR images), and/or two-dimensional color scan data (e.g., 2D color images). Processing logic can process the three-dimensional scan data, the two-dimensional near infrared scan data, and/or the two-dimensional color scan data to determine the gingival recession measurement. In some embodiments, processing logic can use at least two of the three-dimensional scan data, the two-dimensional near infrared scan data, or the two-dimensional color scan data together generate the 3D color model used to determine the gingival recession measurement and/or categorization.

In some embodiments, processing logic can generate a 3D model of the dentition of the patient based on data plurality of intraoral scans and/or 2D images included in the intraoral scan data.

At block 504, processing logic segments the intraoral scan data into a plurality of oral structures. The oral structures can include a tooth in the dentition of the patient, a gingiva surrounding the tooth, and/or a representation of the intersection between a first portion of the tooth (e.g., cementum) and a second portion of the tooth (e.g., enamel). In some embodiments, the representation on the intersection can be the CEJ of the tooth. In some embodiments, the plurality of oral structures can include the enamel and the cementum, and processing logic can identify the CEJ based on the intersection of the cementum and the enamel. In some embodiments, the plurality of oral structures can include the tooth and the gingiva, and processing logic can identify the gingival line for each tooth based on the tooth and gingiva segmentation information. Alternatively, the machine learning model that performs segmentation may specifically generate segmentation information on the margin line (e.g., may output segmentation information on the margin line in addition to or instead of the segmentation information on the CEJ). In some embodiments, processing logic can perform the functions of input segmentation engine 213 of FIG. 2.

In some implementations, to segment the intraoral scan data, at block 506 processing logic provides, as input to a trained machine learning model, the intraoral scan data, and receives, as output from the trained machine learning mode, segmented scan data (e.g., segmentation information) indicating the plurality of oral structures. In some embodiments, the segmented scan data includes instance segmentation for the various oral structures in the intraoral scan data. The segmented scan data may include, for example, a pixel-level mask for each instance of an identified object. For example, pixel-level masks may be generated for each tooth, CEJ, gingiva, gingival line, cementum, enamel, etc. In some embodiments, processing logic can implement a segmenter 615 and/or a segmentation ML model 664 as described with respect to FIG. 6 to segment the intraoral scan data input the plurality of structures.

At block 508, processing logic determines a gingival recession measurement indicative of a distance between the gingiva and the intersection of the enamel and cementum (e.g., the CEJ) for each tooth in the intraoral scan data. In some embodiments, the gingival recession measurement represents an apical measurement between the gingiva the intersection. In some embodiments, to determine the gingival recession measurement, processing logic can compare the distance between the gingiva and the intersection measured at a plurality of points along the intersection. The gingival recession measurement for a tooth can be the greatest measured distance for that tooth. In some embodiments, processing logic can perform the functions of gingival recession measurement and categorization engine 220 of FIG. 2 to determine the gingival recession measurement.

In some implementations, to determine the gingival recession measurement, processing logic provides, as input to a trained machine learning model, the segmented intraoral scan data, and receives, as output from the trained machine learning model, the measurement indicative of the distance between the gingiva and the intersection (e.g., CEJ). In some embodiments, processing logic can implement GR measurement ML model 665 as described with respect to FIG. 6 to determine the gingival recession measurement.

At block 510, processing logic provides, to a user device (e.g., device 160 of FIG. 1), the gingival recession measurement. In some embodiments, processing logic can provide the user device (e.g., user device 160 of FIG. 1), the 3D model of the dentition of the patient, together with at least one of the gingival recession measurement or the treatment recommendation. In some embodiments, processing logic can overlay the occlusion data, the gingival recession measurement, the gingival recession categorization, and/or the treatment recommendation on the 3D model for presentation on the user device. In some embodiments, processing logic can display the gingival recession measurements on a 2D image of the patient's dental arch (e.g., a radiograph) to show areas of vertical bone loss (often associated with V-shaped recession caused by occlusal trauma).

Referring to FIG. 5B, at block 552, processing logic identifies a shape of a line separating the gingiva from the first portion of the tooth along the facial surface of the tooth, wherein the first portion of the tooth represents the cementum of the tooth. That is, processing logic identifies a shape of the gingival line (e.g., as illustrated by gingival line 702 of FIG. 7).

In some implementations, to identify the shape of the gingival line, at block 554 processing logic provides the segmented intraoral scan data as input to a trained machine learning model. Process logic receives, as output from the trained machine learning model, the shape of the line separating the gingiva from the first portion of tooth along the facial surface of the tooth.

In some implementations, to identify the shape, at block 556, processing logic measures a distances between the gingiva and the CEJ at a plurality of points along the CEJ. Processing logic may then compare the distances for the different points on the CEJ. In some embodiments, processing logic determines differences between distances of the CEJ to the margin line for multiple points on the CEJ and uses the differences to assess gingival recession shape. In response to determining that a difference between the distance between the CEJ and the margin line at two consecutive points satisfies a criterion, processing logic identifies the shape as a first shape corresponding to the criterion. For example, processing logic can measure the distance between the gingiva and the intersection at each millimeter along the CEJ. If the difference between two consecutive distance measurements is more than a predetermined value (e.g., more than 2 millimeters), processing logic can determine that the line is a “V” shape. If none of the differences between consecutive distance measurements is more than a threshold amount (e.g., more than 2 millimeters), processing logic can determine that the line is a “U” shape. Note that this is an illustrative example, and other numerical values and/or criteria can be used to identify the shape of “V” or “U” shaped.

At block 558, processing logic identifies, based at least in part on the shape of the line, a cause of the gingival recession for the patient. For example, the cause of a “U” shaped gingival recession can be linked to poor dental hygiene, while a “V” shaped gingival recession can be linked to malocclusion.

At block 560, processing logic determines a treatment recommendation based at least in part on the shape of the line separating the gingiva from the first portion of the tooth (e.g., the cementum). For example, for a “U” shaped gingival recession, processing logic can provide a treatment recommendation that includes proper dental hygiene habits. As another example, for “V” shaped gingival recession, processing logic can receive occlusion data associated with the patient, and processing logic can analyze the occlusion data to determine a treatment recommendation. For example, processing logic can analyze the occlusion data to identify an area of high collision in the mouth, which may be identified as the cause for the “V” shaped recession. The treatment recommendation can include orthodontic treatment (e.g., aligners) to treat the malocclusion.

In some embodiments, processing logic maintains a data store that includes a plurality of gingival recession measurements for the patient (e.g., in data store 108). The plurality of gingival recession measurements can be generated over a period of time, thus recording the longitudinal progression of the gingival recession. The plurality of the gingival recession measurements include the gingival recession measurement indicative of the distance between the gingiva and the first portion of the tooth (e.g., the CEJ). Processing logic can determine, based on the plurality of gingival recession measurements for the patient, a gingival recession progression over one or more periods of time. The treatment recommendation can be further based on the gingival recession progression over the period(s) of time. For example, for gingival recession that progresses quickly over a period of time (e.g., the difference in corresponding recession measurements taken at two points in time exceeds a threshold), processing logic can identify a more aggressive treatment recommendation (e.g., orthodontia to correct malocclusion, or a modification to a current orthodontic treatment plan to prevent further progression of the recession). For gingival recession that progresses more slowly over the period of time (e.g., the difference in corresponding recession measurements taken at two points in time is less than a threshold), processing logic can identify a less aggressive treatment recommendation (e.g., a night guard to prevent further teeth movement).

At block 562, processing logic provides, to the user device(e.g., device 160 of FIG. 1), the treatment recommendation. In one embodiment, processing logic outputs treatment recommendations to a display via a GUI of gingival recession measurement and categorization system 115 of FIGS. 1, 2.

FIG. 6 illustrates workflows for training and using one or more machine learning models to perform gingival recession measurement and categorization, in accordance with some embodiments of the present disclosure. The illustrated workflows include a model training workflow 605 and a model application workflow 617. The model training workflow 605 is to train one or more machine learning models (e.g., deep learning models, generative models, etc.) to perform one or more image segmentation tasks and/or provide measurements and/or categorization of gingival recession. The model application workflow 617 is to apply the one or more trained machine learning models to segment input images and/or provide measurements and/or categorization of gingival recession.

One type of machine learning model that may be used is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, gingiva, enamel, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

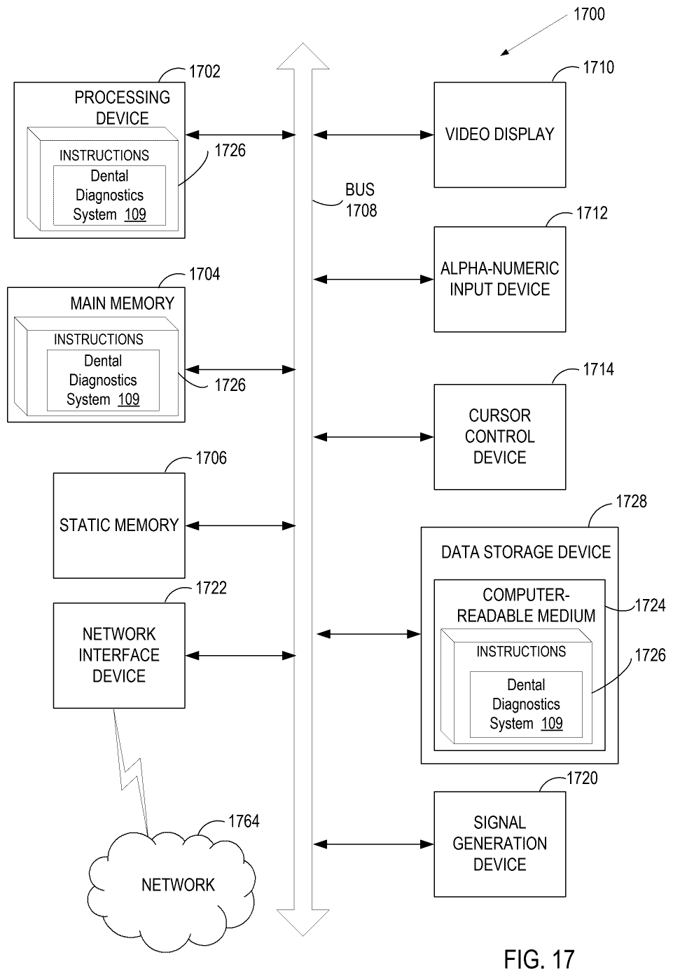

The model training workflow 605 and the model application workflow 617 may be performed by processing logic executed by a processor of a computing device (e.g., computing device 105 of FIG. 1 or a separate computing device). These workflows 605, 617 may be implemented, for example, by one or more modules executed on a processing device 1702 of computing device 1700 shown in FIG. 17.

For the model training workflow 605, training dataset 610 containing hundreds, thousands, tens of thousands, hundreds of thousands, or more images (e.g., scan data) may be provided. Training dataset 610 can include 3D intraoral scan data with labels, 3D virtual models with labels, 2D intraoral scan data with labels, 2D images with labels, and/or additional data with labels. The additional data with labels can include, for example, occlusion data, color data, patient data, and/or other relevant data. In some embodiments, training dataset 610 can include labeled 3D color models generated from intraoral scan data of the dentition of a patient and/or color 2D images.

In some embodiments, some or all of the scan data may be labeled with segmentation information, gingival recession information (e.g., gingival recession measurements, gingival line shape, etc.), and/or other information. The segmentation information may identify features such as individual teeth (optionally including tooth number), gingiva, cementum, enamel, and/or CEJ.

In some embodiments, scan data in training dataset 610 is processed by a segmenter 615 that segments the scan data into multiple different features (e.g., oral structures such as teeth, gingiva, etc.), and that outputs segmentation information for the scan data. The segmenter 615 may be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, tooth number, gingiva, cementum, enamel, and/or CEJ for each tooth. The segmenter 615 may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects. In one embodiment, a single mask or map includes segmentation information for all identified classes of features and/or objects. Some types of features are location-specific features and are represented in one or more masks.

In some embodiments, the segmenter 615 performs one or more image processing and/or computer vision techniques or operations to extract segmentation information from images. Such image processing and/or computer vision techniques may or may not include the use trained machine learning models. Accordingly, in some embodiments, segmenter 615 does not include a machine learning model. Some examples of image processing and/or computer vision techniques that may be performed by segmenter 615 includes determining a color distribution of each tooth, which can be used to identify cementum and enamel, and thus the CEJ.

At block 638, scan data from the training dataset 610 and segmentation information 618 may be used to train one or more machine learning models to measure and/or categorize gingival recession. The training dataset containing hundreds, thousands, tens of thousands, hundreds of thousands, or more data points can be used to form the training dataset 610 and optionally including segmentation information 618. In embodiments, up to millions of scan data and segmentation information are included in a training dataset.

Training may be performed by inputting one or more scan data points and corresponding segmentation information into the machine learning model one at a time. The data that is input into the machine learning model may include a single layer or multiple layers. In some embodiments, a recurrent neural network (RNN) is used. In such an embodiment, a second layer may include a previous output of the machine learning model (which resulted from processing a previous input).

The machine learning model processes the input to generate an output. An artificial neural network includes an input layer that consists of values in a data point. The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce. For example, for an artificial neural network being trained to output gingival recession measurement and/or categorization for each tooth.

Processing logic may then compare the generated measurements and/or categorizations to the known condition and/or label that was included in the training data item. Processing logic determines an error based on the differences between the output probability map and/or label(s) and the provided probability map and/or label(s). Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons,” where each layer receives input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the model. After one or more rounds of training, processing logic may determine whether a stopping criterion has been met. A stopping criterion may be a target level of accuracy, a target number of processed data items from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset 610 may be used to test the model. Testing the model can include performing unit tests, regression tests, and/or integration tests.

Once one or more trained ML models are generated, they may be stored in model storage 645. Multiple ML models can be trained and used in combination. For example, model training workflow 605 can train a GR measurement ML model and a GR categorization ML model. GR measurement ML model can output one or more values of gingiva recession measurement for each tooth, and GR categorization ML model can output a categorization of the gingival recession for each tooth (e.g., “U” shaped or “V” shaped).

In some embodiments, model application workflow 617 includes one or more trained machine learning models that function as segmentation ML model 664, gingival recession (GR) measurement ML model 665, and/or GR categorization model 666. These logics may be implemented as separate machine learning models or as a single combined machine learning model in embodiments. For example, segmentation ML model 664, GR measurement ML model 665, and/or GR categorization ML model 666 may share one or more layers of a deep neural network. However, each of these logics may include distinct higher level layers of the deep neural network that are trained to generate different types of outputs.

A dental professional (e.g., doctor, dentist, hygienist, or technician) may capture an intraoral scan of a patient, which may correspond to intraoral scan(s) 648. The dental professional may have previously captured an intraoral scan of the patient, and/or may have other patient data, such as the patient's chart, the patient's previous gingival recession measurements and/or categorizations, and/or a patient's occlusogram, which may correspond to patient data 654. The intraoral scan data 648 and/or patient data 648 may include 2D images, 3D images, frames of a 2D video, frames of a 3D video, etc. Intraoral scan data 648 and patient data 654 may be combined to form input data 662. Input data 662 may be processed by segmentation ML model 664. In some embodiments, segmentation ML model 664 may perform the same functions as segmenter 615. Segmentation ML model 664 may produce output 668, which can include segmentation information identifying gingiva, each tooth of the patient's dentition (including, e.g., a tooth number), the gingival line of each tooth, the cementum of each tooth, the CEJ of each tooth, and/or the enamel of each tooth.

Output 668 can be provided as input to GR measurement ML model 665 and/or GR categorization model 666. GR measurement ML model 665 may produce output 670, which may include measurements of the gingival recession for each tooth in the patient's dentition. If the CEJ is not visible on a particular tooth's facial surface, the GR measurement ML model 665 may output a value indicating no gingival recession (e.g., a positive number). If the CEJ is visible on a particular tooth's facial surface, the GR measurement ML model 665 may output a value indicating the apical distance between the gingival line and the CEJ for the particular tooth. In some embodiments, the GR measurement ML model 665 may output a series of measurements indicating the distance between the gingival line and the CEJ at various points along the facial surface of each tooth.

GR categorization ML model 666 may produce output 672, which may include a classification of the gingival recession for each tooth. The classification can be, for example, a “U” shape or a “V” shape. Output aggregator 676 may aggregate output 670 and output 672 to produce aggregated output 678. Thus, the model application workflow 617 may produce, as aggregated output, information indicating the gingival recession measurement and categorization for each tooth identified in the intraoral scan of the patient's dentition.

FIG. 7 illustrates U-shaped and V-shaped gingival recession, in accordance with some embodiments of the present disclosure. FIG. 7 illustrates two groups of three teeth. The gingival margin 702 indicates the most coronal edge of the gingiva that surrounds the teeth. Gingival recession is present when the gingival margin moves away from the tooth surface and exposes the cementum 712. The cementum 712 is calcified substance that covers the root of a tooth. Thus, if the cementum 712 is observed on a tooth, the gingiva has moved away from the tooth and root of the tooth is exposed. The cementum has a yellow color, while the enamel has more of a white color. The enamel 714 is the protective, outer covering of the tooth. The cementum 712 joins the enamel 714 to form the cementoenamel junction (CEJ) 703. The gingival recession distance measurement 710 is the distance between the gingival margin 702 and the CEJ 703. “V” shaped gingival recession 701 can be indicative of a misalignment of the teeth, which can require orthodontic treatment (e.g., aligners). “U” shaped gingival recession 711 can be indicative of an oral hygiene issue, which can be addressed through patient education on cleaning and overall oral hygiene.

Some embodiments are discussed herein with reference to dental treatment, such as orthodontic treatment. However, it should be understood that embodiments discussed with reference to dental treatment plans also apply to other medical treatment plans, such as other types of multi-stage medical treatment plans where there are multiple stages that require some active step and/or monitoring (e.g., by the patient, by an automated system) to advance to another (e.g., subsequent) stage.

Furthermore, some embodiments are discussed herein with reference to orthodontic treatment plans that may include the use of orthodontic aligners (also referred to simply as aligners). As used herein, an aligner is an orthodontic appliance that is used to reposition teeth. In some embodiments, orthodontic appliances, such as aligners, impart forces to the crown of a tooth and/or an attachment positioned on the tooth at one or more points of contact between a tooth receiving cavity of the appliance and received tooth and/or attachment. The magnitude of each of these forces and/or their distribution on the surface of the tooth can determine the type of orthodontic tooth movement which results.

Tooth movements may be in any direction in any plane of space, and may comprise one or more of rotation or translation along one or more axes. Types of tooth movements include extrusion, intrusion, rotation, tipping, translation, and root movement, and combinations thereof, as discussed further herein. Tooth movement of the crown greater than the movement of the root can be referred to as tipping. Equivalent movement of the crown and root can be referred to as translation. Movement of the root greater than the crown can be referred to as root movement.

It should be noted that embodiments also apply to other types of dental treatment that may incorporate use of one or more other dental and/or orthodontic appliances including but not limited to brackets and wires, retainers, palatal expanders, and/or other functional appliances. Accordingly, it should be understood that any discussion of aligners herein also applies to other types of orthodontic and/or dental appliances.

FIG. 8 illustrates a diagram of an example TMD diagnostics system 116, in accordance with some embodiments of the present disclosure.

In some embodiments, TMD diagnostics system 116 can include an input preprocessing engine 312 and/or a TMD detection/diagnostics engine 320. In some embodiments, input preprocessing engine 312 and/or a TMD detection/diagnostics engine 320 can perform the same functions as input preprocessing engine 312 and/or a TMD detection/diagnostics engine 320 described with respect to FIG. 3.

Input preprocessing engine 312 can be a software program hosted by a device (e.g., computing device 105) to preprocess received data (e.g., scan data 351, audio data 352, and/or video data 353 of FIG. 3). Input preprocessing engine 312 can include an image segmentation engine 813, a video stabilization engine 815, and/or an audio processing engine 817.

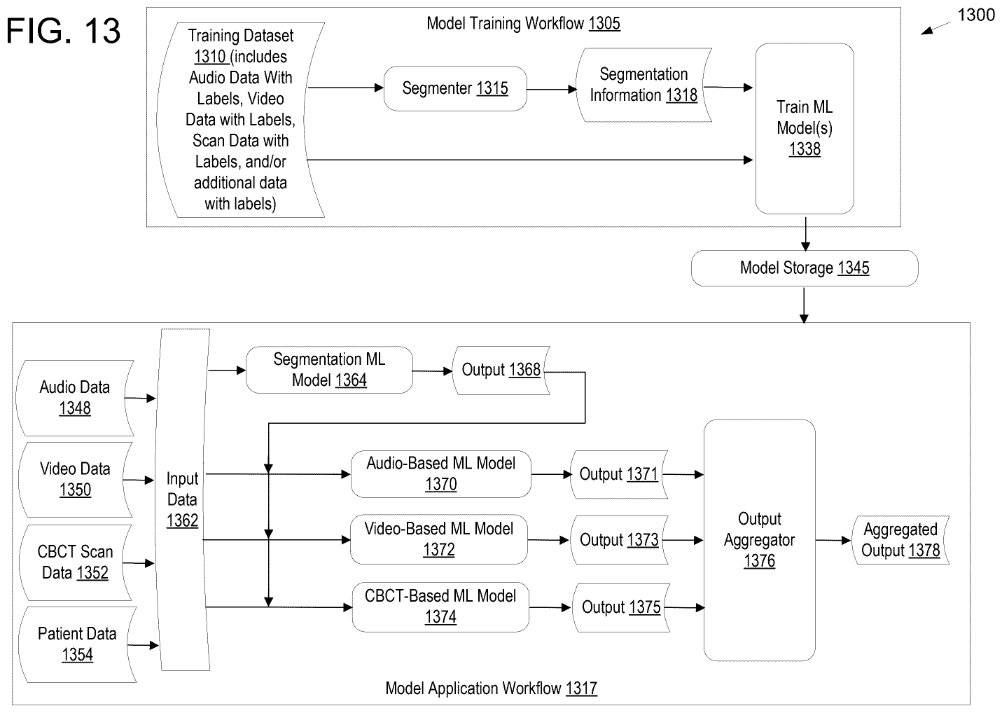

In some embodiments, image segmentation engine 813 can be a software program hosted by a device (e.g., computing device 105) to segment image data (e.g., frames of video data 353, and/or images of CBCT scan data 351 of FIG. 3). Image segmentation engine 813 can segment scan data 351 (e.g., 2D images, intraoral scans, 3D models, etc.) and/or video data 353 into features, such as mandible, teeth, the TMJ's cartilage disc, etc. In some embodiments, the image segmentation engine 813 can receive scan data 351 of a patient's jaw (e.g., CBCT scan data). The image segmentation engine 313 can include a trained machine learning model that takes scan data as input, and outputs segmentation data indicating the jaw features (e.g., to determine sizes, shapes, locations, etc. of the mandible, teeth, the TMJ's cartilage disc, etc.). In some embodiments, image segmentation engine 813 can correspond to segmenter 1315 and/or segmentation ML model 1364 of FIG. 13. Generated segmentation information may be stored as in segmentation data 354 of FIG. 3, in embodiments.

In some embodiments, image segmentation engine 813 can segment video data 353 (e.g., one or more frames of a video stored in video data 353). In some embodiments, the image segmentation engine 813 can receive video data 353, which can include a video recording of a patient opening and/or closing their mouth. In some embodiments, the image segmentation engine 813 can receive one or more frames of a video of a recording of a patient opening and/or closing their mouth. In some embodiments, input preprocessing engine 312 can identify frames from a video data 353. The image segmentation engine 813 can include a trained machine learning model that takes frame data as input, and outputs segmentation data including the jaw features (e.g., to determine sizes, shapes, locations, etc. of the mandible, teeth, the TMJ's cartilage disc, etc.) in one or more frames. In some embodiments, image segmentation engine 813 can correspond to segmenter 1315 and/or segmentation ML model 1364 of FIG. 13. Generated segmentation information may be stored as in segmentation data 354 of FIG. 3, in embodiments.

Applicant hereby incorporates by reference the following application as if set forth fully here, as an example of a machine learning dental segmentation system and method, and training of such a machine learning segmentation system: U.S. Pat. Pub. No. 20210196434A1.

In some embodiments, video stabilization engine 815 can be a software program hosted by a device (e.g., computing device 105) to stabilize video data (e.g., video data 353 of FIG. 3). Stabilizing the video may make movements of the jaw easier to identify. In some embodiments, video stabilization engine 815 can perform one or more video processing and/or artificial intelligence techniques or operations to stabilize the frames of video data 353 to a fixed point (e.g., to a fixed point of the patient's head or skull). Thus, in some embodiments, video stabilization engine 815 can stabilize the position and/or orientation of the patient's head, such that the video data 353 is adjusted so that the patient's head is at a fixed location in the video frames, e.g., regardless of the movement of the camera or the movement of the head. In some embodiments, video stabilization engine 815 can stabilize the position and/or orientation of the camera, such that the video data 353 is adjusted to stabilize so that the camera is at a fixed location (e.g., as if on a tripod) even if the camera was moving when video data 353 was captured.

In some embodiments, video stabilization engine 815 can identify one or more relevant objects in a frame of the video that is also present in another frame. Video stabilization engine 815 can identify one or more patches (e.g., 8×8 or 16×16 blocks of pixels) of a relevant object in a first frame N. To stabilize the position and/or orientation of the patient's head, the relevant object can be, for example, a portion of the patient's head (exclusive the jaw). To stabilize the position and/or orientation of the camera, the relevant object can be, for example, an object in the background (e.g., a non-person object). In some embodiments, the first frame N can be the first frame of the video data 353. In some embodiments, video stabilization engine 815 can identify the first frame N as the first frame in which movement is detected. Video stabilization engine 815 can then identify the relevant object(s) is a subsequent frame N+1, and can determine the movement of the relevant object(s) by subtraction the location of the relevant object at frame N from the location at frame N+1. Video stabilization engine 815 identify the location of multiple relevant objects, and can determine a field of motion vectors using the location of the relevant objects at various points in time (e.g., time of frame N, time of frame N+1, etc.). Video stabilization engine 815 can use the field of motion vectors to compute a transform that maps Frame N+1 into the same stabilized coordinated system as frame N. Transformations can be bilinear, bicubic, biquadratic, affine, or a homography. Thus, video stabilization engine 815 can reposition each subsequent frame of the video to stabilize the identified relevant object(s). In some embodiments, video stabilization engine 815 can be or include a trained machine learning model that receives video data 353 as input, and outputs a stabilized video data as output.

In some embodiments, audio processing engine 817 can be a software program hosted by a device (e.g., computing device 105) to process audio data (e.g., audio data 353 of FIG. 3). Audio processing engine 817 can perform a digital preprocessing of audio data 353. Audio processing engine 817 can implement convention filtering techniques filter out background noise (e.g., the noise of scanning device). In some embodiments, audio processing engine 817 can extract frequency data from the audio data 353, and can identify a frequency range that corresponds to sound(s) of TMD, and one or more frequency range that do not correspond to the sound(s) of TMD. The frequency range that corresponds to sound(s) of TMD may differ based on whether the audio data was captured intraorally or outside of the oral cavity (e.g., whether the audio data is from an intraoral scanner, or whether the audio data is from a microphone held outside of the patient's oral cavity). The audio processing engine 817 can amplify the frequency range corresponding to TMD, and/or can reduce or remove the frequency range(s) that do not correspond to the sound(s) of TMD. In some embodiments, audio processing engine 817 can use Fourier transform, fast Fourier transform, wavelet decomposition, and/or other techniques.

In some embodiments, the audio processing engine 817 can be configured to identify frequency ranges that are associated with sounds of TMD. For example, audio signals that correspond to TMJ clicking events, such as those resulting from anterior disc displacement with reduction, are found in the lower frequency range, typically below approximately 300 Hz. These lower-frequency clicks are often characterized by a temporal duration of 2 to 20 milliseconds and may be more readily detected in both intraoral and extraoral microphones. In contrast, crepitus sounds, which can be indicative of degenerative joint changes or arthrosis, are characterized by a series of short-duration, high-frequency events with substantial energy content above 300 Hz, and in many cases, significant components are observer above 3,000 Hz. Intraoral microphones or accelerometers, due to their proximity to the TMJ and reduced tissue damping, can be effective at capturing these high-frequency components above 3,000 Hz, whereas extraoral microphones may experience attenuation of such frequencies but remain effective for detecting clinically relevant ranges below 300 Hz or above 300 Hz. Frequency ranges that do not correspond to TMD sounds can include those below approximately 20 Hz, which are typically associated with movement artifacts or environmental noise, as well as certain mid-frequency ranges (e.g., 300 Hz to 3,000 Hz) that may contain normal joint movement or background noise, depending on the recording context. Accordingly, the audio processing engine 817 may be configured to extract and/or amplify frequency ranges below 300 Hz for clicking events and/or above 300 Hz (especially above 3,000 Hz) for crepitus sounds to enhance the detection of TMD-related sounds, while attenuating or filtering out frequencies below 20 Hz and those not exhibiting the characteristic spectral patterns of pathological TMJ activity. It should be understood that additional frequency ranges may be associated with sounds indicative of TMD, and that the audio processing engine 817 can be configured to amplify and/or filter out such additional frequency ranges to enhance the detection and analysis of TMD-related audio signals.

In some embodiments, TMD detection/diagnostics engine 320 can be a software program hosted by a device (e.g., computing device 105) to detect, assess, and/or diagnose TMD of a patient. TMD detection/diagnostics engine 320 can include an audio-based TMD detection engine 822, a video-based TMD detection engine 824, and/or a CBCT-based TMD detection engine 826. In some embodiments, one or more of these engines 822-826 may be combined in a single engine.

In some embodiments, audio-based TMD detection engine 822 can be or include a machine learning model that is trained to receive, as input, audio data. The audio-based TMD detection engine 822 can output a value indicating a likelihood of TMD in the audio data. In some embodiments, the value can be between 0 and 1 (inclusive), with a higher value indicating a higher likelihood of TMD. The audio data can be audio data 352 of FIG. 3. In some embodiments, audio data can be preprocessed by audio processing engine 817, e.g., to remove background noise, amplify the frequency range that corresponds to TMD sounds, reduce or remove the frequency range(s) that do not correspond to TMD sounds, etc. In some embodiments, audio-based TMD detection engine 822 can include a set of rules (e.g., corresponding to classification data 355) to classify the audio data 353. Sounds indicating a likelihood of TMD include, for example, clicking, popping, and/or crepitus during opening, lateral, and/or protrusive movements of the patient's jaw.

In some embodiments, video-based TMD detection engine 824 can be or include a machine learning model that is trained to receive, as input, video data. The video-based TMD detection engine 824 can output a value indicating a likelihood of TMD in the video data. In some embodiments, the value can be between 0 and 1 (inclusive), with a higher value indicating a higher likelihood of TMD. The video data can be video data 353 of FIG. 3. In some embodiments, video data can be preprocessed by input preprocessing engine 312, e.g., to generate individual frames of the video data, to stabilize the video, to segment each frame, etc.

In some embodiments, video-based TMD detection engine 824 can perform one or more image processing and/or computer vision techniques or operations to track the location of the jaw during movement. In some embodiments, video-based TMD detection engine 824 can be or include a trained machine learning model that processes a stream of images and detects motion indicative of TMD. The motion indicative of TMD can include, for example, catching, snapping and/or popping during opening, lateral, and/or protrusive movements of the patient's jaw. In some embodiments, video-based TMD detection engine 824 can identify the patient's skull and mandible in two or more consecutive frames (e.g., as identified by segmentation engine 813). The video-based TMD detection engine 824 can measure the distance between the skull and the mandible in each of the two or more consecutive frames. If the difference between distance between the skull and the mandible of two consecutive frames exceeds a threshold, the video-based TMD detection engine 824 can determine a likelihood of the presence of TMD. In some embodiments, if the difference between the distance between the skull and the mandible in two consecutive frames is greater than the distance measured in the other consecutive frames of the video data, the video-based TMD detection engine 824 can determine a likelihood of the presence of TMD.

As an illustrative example, in the first frames of the video, the difference between the distance between the skull and the mandible in each set of consecutive frames may be 2 millimeters (mms) (e.g., the mandible moves at a rate of 2 mms per frame). At a certain point, the difference between the skull and the mandible in two consecutive frames is 5 mms (e.g., the mandible moves at a rate of 5 mms per frame). The video-based TMD detection engine 824 can compare the difference to a threshold (e.g., the 5 mms per frame movement), and/or can compare the difference to the previous measurements (e.g., compare the 5 mms per frame movement to the previously measured 8 mms per frame movement), to determine a likelihood of the presence of TMD. Note that the distance can be measured in pixels, or in any other appropriate measurement unit.

In some embodiments, video-based TMD detection engine 824 can measure the maximum opening of the patient's mouth. For example, the video-based TMD detection engine 824 can measure the distance between the jaw and the skull in each frame of video data 353, and can determine the greatest measured distance. The video-based TMD detection engine 824 can identify a likelihood of TMD if the greatest measured distance is less than a threshold value.

In some embodiments, the measured opening or distance is measured in pixels. In order to determine the measurement in units of physical measurement, the image may be registered with 3D model(s) of the patient's upper and/or lower dental arches. The 3D model(s) may include accurate size information of the patient's dental arches in units of physical measurement (e.g., in mm). Based on the registration of the images (e.g., video frames) to the 3D model(s), a conversion factor for converting between units of digital measurement and units of physical measurement may be determined. The conversion factor may be applied to the measured opening or distance in units of digital measurement (e.g., pixels) to determine the opening or distance in units of physical measurement (e.g., mm or inches).

In some embodiments, CBCT-based TMD detection engine 826 can be or include a machine learning model that is trained to receive, as input, CBCT scan data. The CBCT-based TMD detection engine 826 can output a value indicating a likelihood of TMD in the CBCT scan data. In some embodiments, the value can be between 0 and 1 (inclusive), with a higher value indicating a higher likelihood of TMD. The CBCT scan data can be CBCT scan data 351 of FIG. 3. In some embodiments, CBCT scan data can be preprocessed by image segmentation engine 813, e.g., to identify features of the scan data.

In some embodiments, CBCT-based TMD detection engine 826 can identify the TMJ bones and other bones in the scan data 351 (e.g., from segmentation data 354). The CBCT-based TMD detection engine 826 can determine the density of the bones of the TMJ, and can compare the density of the bones of the TMJ to other identified bones in the scan data 351. If the bone density in the TMJ is less than the density of the other bones in the scan data 351, the CBCT-based TMD detection engine 826 can determine a likelihood of the presence of TMD. In some embodiments, the CBCT-based TMD detection engine 826 can identify the fossa, the disc, the teeth, and/or other features of the scan data 351 (e.g., from segmentation data 354). The CBCT-based TMD detection engine 826 can determine a likelihood of TMD based on an abnormal size, shape, and/or appearance of the TMJ bones (e.g., condylar head, fossa/articular eminence), and/or incorrect relation/position of the condyle to the articular fossa. For example, a CBCT-scan taken as the patient has their mouth fully open can be used to identify an abnormal position of the disc. In some embodiments, the CBCT-based TMD detection engine 826 can evaluate osseous components (e.g., bones) to determine a likelihood of the presence of TMD. For example, CBCT-scan data can be used to identify degenerative joint/bone disease, such as bone resorption (e.g., bone does not look smooth in the scan data).

In some embodiments, CBCT-based TMD detection engine 826 can measure the maximum opening of the patient's mouth, e.g., when the CBCT scan is performed as the patient's mouth is fully opened. The opening of the patient's mouth can be measured as the distance between the mandible and the skull. The CBCT-based TMD detection engine 824 can identify a likelihood of TMD if the greatest measured distance is less than a threshold value.

Image segmentation engine 813, video stabilization engine 815, audio-based TMD detection engine 822, video-based TMD detection engine 824, and CBCT-based TMD detection engine 826 are further described with respect to FIG. 13.

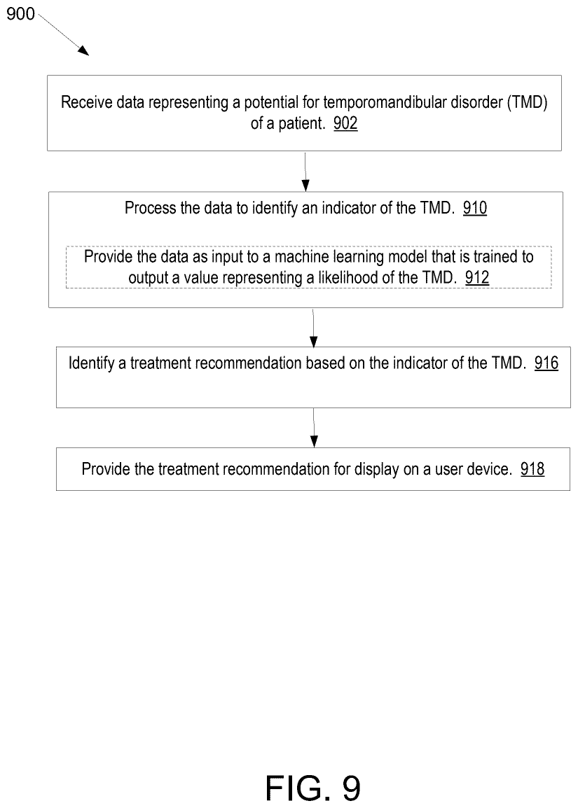

FIGS. 9-12 illustrates flow diagram of example methods 900-1200 for detecting and/or assessing TMD in a patient, in accordance with some embodiments of the present disclosure. One or more of methods 900-1200 may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, one or more of methods 900-1200 may be performed by the processing devices and the associated algorithms, e.g., as described in conjunction with FIGS. 1, 3. In embodiments, one or more of methods 900-1200 is performed by processing logic comprising hardware, software, firmware, or a combination thereof. In certain embodiments, one or more of methods 900-1200 may be performed by a single processing thread. Alternatively, one or more of methods 900-1200 may be performed by two or more processing threads, each thread executing one or more individual functions, routines, subroutines, or operations of the method. In an illustrative example, the processing threads implementing one or more of methods 900-1200 may be synchronized (e.g., using semaphores, critical sections, and/or other thread synchronization mechanisms). Alternatively, the processing threads implementing one or more of methods 900-1200 may be executed asynchronously with respect to each other. Therefore, while FIGS. 9-12 and the associated descriptions list the operations of methods 900-1200 in a certain order, in some embodiments, at least some of the described operations may be performed in parallel and/or in a different order. In some embodiments one or more operations of one or more of methods 900-1200 is not performed.

FIG. 9 illustrates flow diagram of an example method 900 for detecting and/or assessing TMD in a patient, in accordance with some embodiments of the present disclosure. At block 902, processing logic can receive data representing sounds of the potential for TMD of a patient (e.g., sounds of the patient opening and/or closing their mouth, or moving their jaw in a lateral or protrusive motion). In some embodiments, the received data can include audio data representing a sound of a potential for TMD of the patient, e.g., recorded by a microphone (e.g., microphone 161 of FIGS. 1, 3) when the patient performs at least one of opening, closing, lateral, or protrusive jaw movements. In some embodiments, the received data can include video data representing a video recording of the patient, e.g., recorded by a camera (e.g., by a camera 162 of FIGS. 1, 3) as the patient performs at least one of opening, closing, lateral, or protrusive jaw movements. in some embodiments, the received data can include a CBCT scan of the patient (e.g., captured by CBCT scanner 163 of FIGS. 1, 3) representing the jaw of the patient in an open-jaw or closed-jaw position. In some embodiments, the received data can be pressure data representing a potential for TMD of the patient. In some embodiments, the received data can be a combination of any of audio, video, CBCT, and/or pressure data.

At block 910, processing logic can process the data to identify an indicator of the TMD. At block 912, processing logic can optionally provide the data as input to a machine learning model that is trained to output a value representing a likelihood of the TMD.

At block 916, processing logic identifies a treatment recommendation based on the indicator of the TMD. At block 918, processing logic provides the treatment recommendation for display on a user device (e.g., device 160 of FIGS. 1, 3). In some embodiments, the treatment recommendation can include an appliance to correct the TMD. For example, the treatment recommendation can include a recommendation to use a mouth-guard (e.g., a custom-made mouth guard), such as an occlusal splint.

In some embodiments, the treatment recommendation can include an aligner treatment, e.g., including an aligner that is designed to reduce one or more symptoms of TMD. In some embodiments, the treatment recommendation can include a recommendation to not implement an aligner treatment, to stop an ongoing aligner treatment, or to slow an aligner treatment (e.g., to extend the amount of time the patient is to wear each aligner). In some embodiments, the treatment recommendation can include an appliance to correct the TMD. In some embodiments, the treatment can include fabricating an appliance based on the indicator of the TMD. The appliance can be a 3D-printed appliance to correct the TMD, and/or a 3D-printed appliance to concurrently treat the TMD and orthodontically move the teeth.

Some embodiments are discussed herein with reference to dental treatment, such as orthodontic treatment. However, it should be understood that embodiments discussed with reference to dental treatment plans also apply to other medical treatment plans, such as other types of multi-stage medical treatment plans where there are multiple stages that require some active step and/or monitoring (e.g., by the patient, by an automated system) to advance to another (e.g., subsequent) stage.

Furthermore, some embodiments are discussed herein with reference to orthodontic treatment plans that may include the use of orthodontic aligners (also referred to simply as aligners). As used herein, an aligner is an orthodontic appliance that is used to reposition teeth. In some embodiments, orthodontic appliances, such as aligners, impart forces to the crown of a tooth and/or an attachment positioned on the tooth at one or more points of contact between a tooth receiving cavity of the appliance and received tooth and/or attachment. The magnitude of each of these forces and/or their distribution on the surface of the tooth can determine the type of orthodontic tooth movement which results.

Tooth movements may be in any direction in any plane of space, and may comprise one or more of rotation or translation along one or more axes. Types of tooth movements include extrusion, intrusion, rotation, tipping, translation, and root movement, and combinations thereof, as discussed further herein. Tooth movement of the crown greater than the movement of the root can be referred to as tipping. Equivalent movement of the crown and root can be referred to as translation. Movement of the root greater than the crown can be referred to as root movement.

It should be noted that embodiments also apply to other types of dental treatment that may incorporate use of one or more other dental and/or orthodontic appliances including but not limited to brackets and wires, retainers, palatal expanders, and/or other functional appliances. Accordingly, it should be understood that any discussion of aligners herein also applies to other types of orthodontic and/or dental appliances.

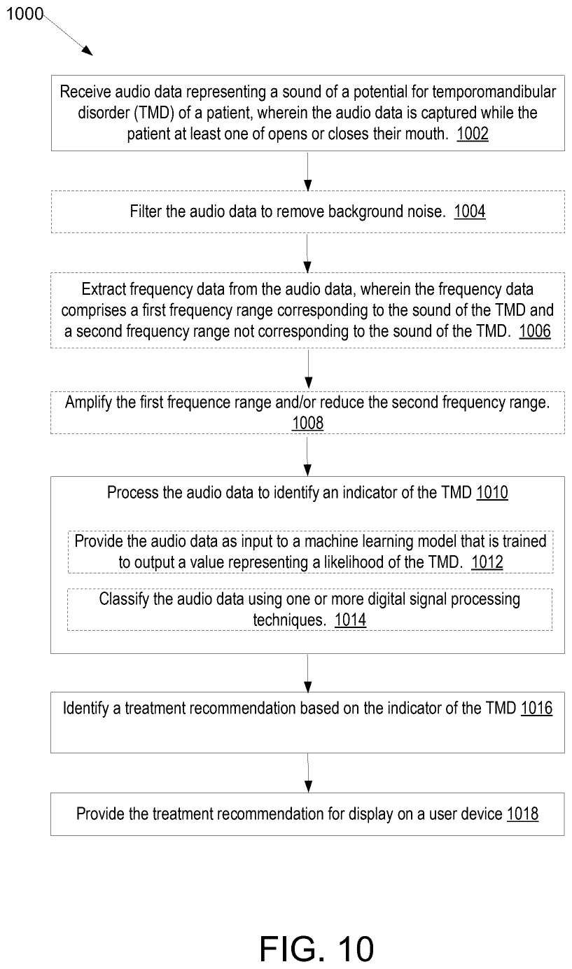

FIG. 10 illustrates a flow diagram of an example method 1000 for detecting and/or assessing TMD in a patient using audio data, in accordance with some embodiments of the present disclosure.

At block 1002, processing logic receives audio data representing a sound of a potential for TMD of a patient. In some embodiments, the audio data is captured (e.g., by a microphone 161 of FIGS. 1, 3) while the patient performs at least one of opening, closing, lateral, or protrusive jaw movements. In some embodiments, processing logic can perform a preprocessing of the audio data (e.g., as described with respect to input preprocessing engine 312 of FIG. 3). In some embodiments, the preprocessing can include blocks 1004-1008. In some embodiments, the preprocessing can include converting the audio data to a spectrogram representing the frequency data over time. In some embodiments, processing logic can process the spectrogram using a trained machine learning model. The trained machine learning model can output the indicator of the TMD. In some embodiments, processing logic can received a recording of the sound of the potential for TMD. The recording can include analog audio signals. Processing logic can convert the recording of the sounds to a digital signal.

At block 1004, processing logic can filter the audio data to remove background noise. At block 1006, processing logic can extract frequency data from the audio data. The frequency data can include a first frequency range corresponding to the sound of the TMD, and a second frequency range not corresponding to the sound of the TMD (e.g., the second frequency range can correspond to background noise). At block 1008, processing logic can amplify the first frequency range and/or reduce the second frequency range. In some embodiments, processing logic can implement a matched filter to maximum the signal-to-noise ratio for a known signal. The known signal can correspond to a set of examples of TMD-related sounds. Processing logic can determine the signal to detect within audio samples, e.g., taken of patients with diagnosed TMD as they open and/or close their mouth, and/or move their jaw laterally or protrusively. Processing logic create a matched filter that is the time-reversed and conjugated version of the target signal. Processing logic can convolve the matched filter with the audio signal of the audio data, and produce a new signal that indicates the presence of the target signal.

At block 1010, processing logic can process the audio data to identify an indicator of the TMD. At block 1012, processing logic can provide the audio data as input to a machine learning model that is trained to output a value representing a likelihood of the TMD (e.g., audio-based ML model 1370 of FIG. 13). In some embodiments, the ML model can identify the sound present in the audio data (e.g., snapping, popping, clicking, crepitus, etc.). In some embodiments, the ML model can output a value between 0 and 1 (inclusive), where a higher value indicates a higher likelihood of the presence of TMD.

At block 1014, processing logic can classify the audio data using one or more digital signal processing techniques. In some embodiments, processing logic can implement matched filters, Wiener filters, spectral methods, Bayesian methods, and/or other digital signal processing techniques to classify the audio data. In some embodiments, processing logic can compare the audio data to known signals that represent sound(s) indicative of TMD to classify the audio data as indicating a likelihood of a presence of TMD. The sound(s) indicative of TMD can include, for example, clicking, snapping, popping, crepitus, etc. In some embodiments, processing logic can classify the audio data as including sound(s) indicative of TMD or not including sound(s) indicative of TMD. Note that the sound(s) indicative of TMD may differ based on whether they were recorded intraorally or from outside the patient's oral cavity.

At block 1016, processing logic can identify a treatment recommendation based on the indicator of the TMD. Treatments can include orthodontic treatment, such as a recommendation to start, modify (e.g., recommend a specific staging for aligners, lighten the elastic forces of orthodontia, increase the wear time per aligner, alternate retention strategies to alleviate the symptoms), or stop orthodontic treatment. As another example, the treatment recommendation may include fabricating an application based on the indication of TMD, e.g., to correct the TMD. In some embodiments, the appliance can be a 3D-printed appliance to correct TMD, or a 3D-printed appliance to concurrently treat the TMD and orthodontically move the teeth. In some embodiments, the treatment recommendation can be based on the severity of the detected TMD, the cause of the TMD, and/or the patient's medical history. For example, if a patient is undergoing orthodontic treatment when TMD symptoms first occur, the treatment recommendation may be to slow the progress of the orthodontic treatment, or to stop the orthodontic treatment if the severity of the symptoms of the TMD exceed a threshold. As another example, if a patient is a candidate for orthodontic treatment but presents with TMD symptoms, and the treatment recommendation may include not starting orthodontic treatment until the TMD has been addressed. The treatment recommendation can be based on a set of rules that take into account the patient's history (e.g., how long the patient has had symptoms, the severity of the symptoms, treatment history, etc.), the severity of the detected TMD, and/or the cause of the TMD. The treatment recommendation can be provided for display on a user device, e.g., along with the assessment, diagnosis, and/or identified cause of the TMD.

In some embodiments, processing logic can receive one or more responses to a patient questionnaire. Processing logic can analyze the one or more responses to identify an additional indicator of the TMD. Processing logic can determine that the patient has the TMD based on a combination of the indicator and the additional indicator.

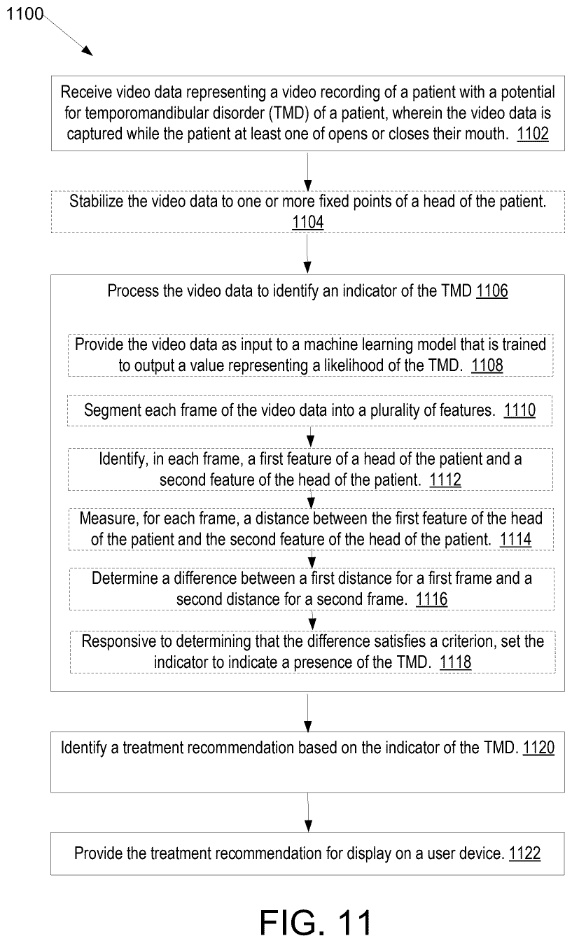

In some embodiments, processing logic receives video data representing a video recording of the patient, captured as the patient performs at least one of opening, closing, lateral, or protrusive jaw movements. Processing logic can process the video data to identify a second indicator of the TMD (e.g., as described with respect to FIG. 11). Processing logic can identify the treatment recommendation further based on the second indicator.

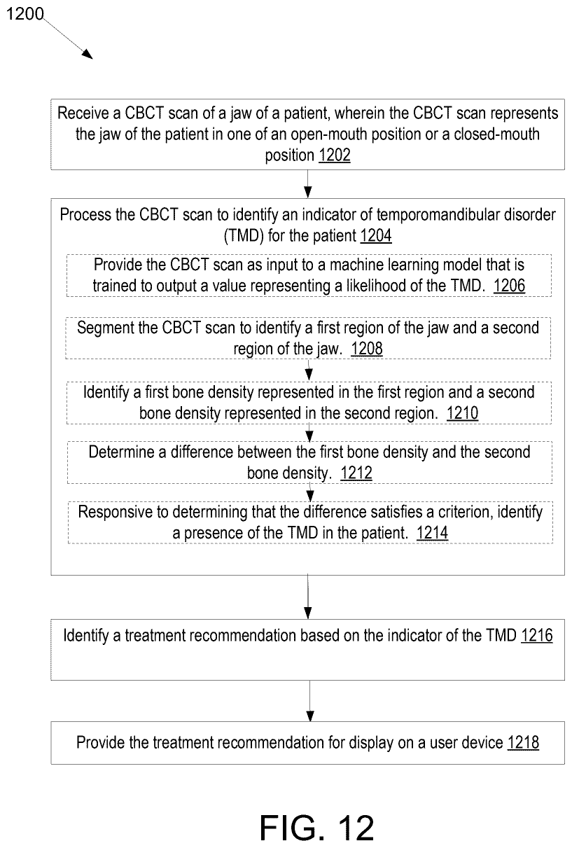

In some embodiments, processing logic can receive a CBCT scan of the patient. Processing logic can analyze the CBCT scan to identify a third indicator of the TMD (e.g., as described with respect to FIG. 12). Processing logic can identify the treatment recommendation further based on the third indicator.

In some embodiments, processing logic can receive video data representing a video recording of the patient captured as the patient performs at least one of opening, closing, lateral, or protrusive jaw movements, and can process the video data to identify a second indicator of the TMD. Processing logic can also receive a CBCT scan of the patient, and analyze the CBCT scan to identify a third indicator of the TMD. Processing logic can identify the treatment recommendation further based on the second and the third indicators.

At block 1018, processing logic can provide the treatment recommendation for display on a user device (e.g., device 160 of FIGS. 1, 3).

FIG. 11 illustrates a flow diagram of an example method 1100 for detecting and/or assessing TMD in a patient using video data, in accordance with some embodiments of the present disclosure.

At block 1102, processing logic receives video data representing a video recording of a patient while a potential for TMD. The video data can be captured (e.g., by camera 162 of FIGS. 1, 3) while the patient performs at least one of opening, closing, lateral, or protrusive jaw movements. At block 1104, processing logic stabilizes the video data to one or more fixed points of a head of the patient. For example, processing logic can identify a reference frame from a set of frames of the video data. The reference frame can be, for example, the first frame of the video data, or can be the first frame in which movement of the patient's jaw is detected (or can be a few frames before movement of the patient's jaw is detected). Processing logic can identify a reference point in the first frame. The reference point can be, for example, a portion of the patient's head (excluding the jaw or mandible), or can be an object in the background (e.g., a non-person object). Processing logic can compute the transformations to alter the subsequent video frames of video data, so that identified reference point is in the same location as seen in the reference frame (e.g., the identified first frame).

At block 1106, processing logic processes the video data to identify an indicator of the TMD. In some embodiments, processing the video data to identify an indicator of the TMD can include performing blocks 1108. In some embodiments, processing the video data to identify an indicator of the TMD can include performing blocks 1110-1118.

At block 1108, processing logic provides the video data as input to a machine learning model that is trained to output a value representing a likelihood of the TMD (e.g., video-based ML model 1372 of FIG. 13). In some embodiments, the machine learning model can process a series of frames (e.g., a stream of images) of the entire video to detect motion indicative of TMD (e.g., by outputting a value representing a likelihood of the TMD). In some embodiments, processing logic can provide one or more pair of consecutive frames from the video as input to the machine learning model that is trained to output a value representing a likelihood of the TMD. In some embodiments, processing logic can identify which pair of consecutive frames showed the change in motion that indicated the likelihood of TMD. In some embodiments, processing logic can segment the frames prior to inputting the frames to the machine learning model. The ML model can then operate on the segmented image data, and output a value representing a likelihood of the TMD.

At block 1110, processing logic segments each frame of the video data into a plurality of features, such as mandible, teeth, the TMJ's cartilage disc, etc. In some embodiments, to segment the video data, processing logic can provide the video data as input to a trained machine learning model. Processing logic can receive, as output form the trained machine learning model, segmented data (e.g., segmentation data 1354 of FIG. 13, segmentation information 1318 of FIG. 13, and/or output 1368 of FIG. 13) indicating the plurality of features. The segmented data may include, for example, a pixel-level mask for each instance of an identified feature. For example, pixel-level masks may be generated for mandible, teeth, TMJ disc, cartilage, etc. In some embodiments, processing logic can implement a segmenter 1315 and/or a segmentation ML model 1364 as described with respect to FIG. 13 to segment the intraoral scan data input the plurality of features.

At block 1112, processing logic identifies, in each frame, a first feature of a head of the patient and a second feature of the head of the patient. In some embodiments, the first feature can be the mandible, and the second feature can be the skull. At block 1114, processing logic measures, for each frame, a distance between the first feature of the head of the patient and a second feature of the head of the patient. At block 1116, processing logic determines a difference between a first distance for a first frame and a second distance for a second frame. In some embodiments, the first frame and the second frame are consecutive frames. At block 1118, responsive to determining that the difference satisfies a criterion, processing logic sets the indicator to indicate a present of the TMD.

At block 1120, processing logic determines a treatment recommendation based on the indicator of the TMD. Treatments can include orthodontic treatment, such as a recommendation to start, modify (e.g., recommend a specific staging for aligners, lighten the elastic forces of orthodontia, increase the wear time per aligner, alternate retention strategies to alleviate the symptoms), or stop orthodontic treatment. As another example, the treatment recommendation may include fabricating an application based on the indication of TMD, e.g., to correct the TMD. In some embodiments, the appliance can be a 3D-printed appliance to correct TMD, or a 3D-printed appliance to concurrently treat the TMD and orthodontically move the teeth. In some embodiments, the treatment recommendation can be based on the severity of the detected TMD, the cause of the TMD, and/or the patient's medical history. For example, if a patient is undergoing orthodontic treatment when TMD symptoms first occur, the treatment recommendation may be to slow the progress of the orthodontic treatment, or to stop the orthodontic treatment if the severity of the symptoms of the TMD exceed a threshold. As another example, if a patient is a candidate for orthodontic treatment but presents with TMD symptoms, and the treatment recommendation may include not starting orthodontic treatment until the TMD has been addressed. The treatment recommendation can be based on a set of rules that take into account the patient's history (e.g., how long the patient has had symptoms, the severity of the symptoms, treatment history, etc.), the severity of the detected TMD, and/or the cause of the TMD. The treatment recommendation can be provided for display on a user device, e.g., along with the assessment, diagnosis, and/or identified cause of the TMD.

In some embodiments, processing logic can receive one or more responses to a patient questionnaire. Processing logic can analyze the one or more responses to identify an additional indicator of the TMD. Processing logic can determine that the patient has the TMD based on a combination of the indicator and the additional indicator.

In some embodiments, processing logic receives audio data representing an audio recording of the patient, captured as the patient performs at least one of opening, closing, lateral, or protrusive jaw movements. Processing logic can process the audio data to identify a second indicator of the TMD (e.g., as described with respect to FIG. 10). Processing logic can identify the treatment recommendation further based on the second indicator.

In some embodiments, processing logic can receive a CBCT scan of the patient. Processing logic can analyze the CBCT scan to identify a third indicator of the TMD (e.g., as described with respect to FIG. 12). Processing logic can identify the treatment recommendation further based on the third indicator.

In some embodiments, processing logic can receive audio data representing an audio recording of the patient captured as the performs at least one of opening, closing, lateral, or protrusive jaw movements, and can process the audio data to identify a second indicator of the TMD. Processing logic can also receive a CBCT scan of the patient, and analyze the CBCT scan to identify a third indicator of the TMD. Processing logic can identify the treatment recommendation further based on the second and the third indicators.

At block 1122, processing logic provides the treatment recommendation for display on a user device (e.g., device 160 of FIGS. 1, 3).

FIG. 12 illustrates a flow diagram of an example method 1200 for detecting and/or assessing TMD in a patient using scan data, in accordance with some embodiments of the present disclosure.

At block 1202, processing logic receives a CBCT scan of a jaw of a patient. In some embodiments, the CBCT scan represents the jaw of the patient in an open-mouth position or a closed-mouth position.

At block 1204, processing logic processes the CBCT scan to identify an indicator of TMD for the patient. In some embodiments, processing the CBCT scan to identify the indicator of the TMD can include block 1206. In some embodiments, processing the CBCT scan to identify the indicator of the TMD can include blocks 1208-1214.

At block 1206, processing logic provides the CBCT scan as input to a machine learning model that is trained to output a value representing a likelihood of the TMD (e.g., CBCT-based ML model 1374 of FIG. 13). In some embodiments, processing logic segments the CBCT scan data using a first machine learning model (e.g., segmentation ML model 1364 of FIG. 13), and then the segmented data is provided as input to a second machine learning model (e.g., CBCT-based ML model 1374 of FIG. 13) to detect a likelihood of the presence of TMD.

At block 1208, processing logic segments the CBCT scan to identify a first region of the jaw and a second region of the jaw. In some embodiments, the first region of the of the jaw can be the teeth, and the second region of the jaw can be the condyle.

In some embodiments, to segment the CBCT scan data, processing logic can provide the CBCT scan data as input to a trained machine learning model. Processing logic can receive, as output form the trained machine learning model, segmented scan data (e.g., segmentation data 1354 of FIG. 13, segmentation information 1318 of FIG. 13, and/or output 1368 of FIG. 13) indicating the plurality of features. The segmented scan data may include, for example, a pixel-level mask for each instance of an identified feature. For example, pixel-level masks may be generated for mandible, teeth, TMJ disc, cartilage, etc. In some embodiments, processing logic can implement a segmenter 1315 and/or a segmentation ML model 1364 as described with respect to FIG. 13 to segment the intraoral scan data input the plurality of features.

At block 1210, processing logic identifies a first bone density represented in the first region and a second bone density represented in the second region. At block 1212, processing logic determines a difference between the first bone density and the second bone density. At block 1214, responsive to determining that the difference satisfies a criterion, processing logic identify a presence of the TMD in the patient.

At block 1216, processing logic identifies a treatment recommendation based on the indicator of the TMD. Treatments can include orthodontic treatment, such as a recommendation to start, modify (e.g., recommend a specific staging for aligners, lighten the elastic forces of orthodontia, increase the wear time per aligner, alternate retention strategies to alleviate the symptoms), or stop orthodontic treatment. As another example, the treatment recommendation may include fabricating an application based on the indication of TMD, e.g., to correct the TMD. In some embodiments, the appliance can be a 3D-printed appliance to correct TMD, or a 3D-printed appliance to concurrently treat the TMD and orthodontically move the teeth. In some embodiments, the treatment recommendation can be based on the severity of the detected TMD, the cause of the TMD, and/or the patient's medical history. For example, if a patient is undergoing orthodontic treatment when TMD symptoms first occur, the treatment recommendation may be to slow the progress of the orthodontic treatment, or to stop the orthodontic treatment if the severity of the symptoms of the TMD exceed a threshold. As another example, if a patient is a candidate for orthodontic treatment but presents with TMD symptoms, and the treatment recommendation may include not starting orthodontic treatment until the TMD has been addressed. The treatment recommendation can be based on a set of rules that take into account the patient's history (e.g., how long the patient has had symptoms, the severity of the symptoms, treatment history, etc.), the severity of the detected TMD, and/or the cause of the TMD. The treatment recommendation can be provided for display on a user device, e.g., along with the assessment, diagnosis, and/or identified cause of the TMD.

In some embodiments, processing logic identifies a third region of the jaw of the patient. Processing logic compares a position of a first portion of the third region to a second portion of the third region. Processing logic determines, based on the comparison, that the position is abnormal. Responsive to determining that the position of the first portion is abnormal, processing logic identifies a presence of the TMD in the patient.

In some embodiments, processing logic can receive one or more responses to a patient questionnaire. Processing logic can analyze the one or more responses to identify an additional indicator of the TMD. Processing logic can determine that the patient has the TMD based on a combination of the indicator and the additional indicator.

In some embodiments, processing logic receives audio data representing an audio recording of the patient, captured as the patient performs at least one of opening, closing, lateral, or protrusive jaw movements. Processing logic can process the audio data to identify a second indicator of the TMD (e.g., as described with respect to FIG. 10). Processing logic can identify the treatment recommendation further based on the second indicator.

In some embodiments, processing logic receives video data representing a video recording of the patient, captured as the patient performs at least one of opening, closing, lateral, or protrusive jaw movements. Processing logic can process the video data to identify a second indicator of the TMD (e.g., as described with respect to FIG. 11). Processing logic can identify the treatment recommendation further based on the second indicator.

In some embodiments, processing logic can receive audio data representing an audio recording of the patient captured as the patient performs at least one of opening, closing, lateral, or protrusive jaw movements, and can process the audio data to identify a second indicator of the TMD. Processing logic can also receive video data representing a video recording of the patient captured as the patient performs at least one of opening, closing, lateral, or protrusive jaw movements, and can process the video data to identify a second indicator of the TMD. Processing logic can identify the treatment recommendation further based on the second and the third indicators.

At block 1218, processing logic provides the treatment recommendation for display on a user device (e.g., device 1360 of FIG. 13).

FIG. 13 illustrates workflows for training and using one or more machine learning models to perform TMD detection, assessment, and/or diagnosis, in accordance with some embodiments of the present disclosure. The illustrated workflows include a model training workflow 1305 and a model application workflow 1317. The model training workflow 1305 is to train one or more machine learning models (e.g., deep learning models, generative models, etc.) to perform one or more image segmentation tasks and/or provide a likelihood of a presence of TMD in a patient. The model application workflow 1317 is to apply the one or more trained machine learning models to segment input images and/or provide a likelihood of a presence of TMD in a patient.

One type of machine learning model that may be used is an artificial neural network, such as a deep neural network. Artificial neural networks generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, gingiva, enamel, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

The model training workflow 1305 and the model application workflow 1317 may be performed by processing logic executed by a processor of a computing device (e.g., computing device 105 of FIGS. 1, 3 or a separate computing device). These workflows 1305, 1317 may be implemented, for example, by one or more modules executed on a processing device 1702 of computing device 1700 shown in FIG. 17.

For the model training workflow 1305, training dataset 1310 containing hundreds, thousands, tens of thousands, hundreds of thousands, or more images (e.g., scan data, video data, audio data, and/or additional patient data) may be provided. Training dataset 1310 can include audio data with labels, video data with labels, scan data with labels, and/or additional data with labels. The additional data with labels can include, for example, occlusion data, color data, patient data, and/or other relevant data. In some embodiments, training dataset 1310 can include labeled 3D color models generated from intraoral scan data of the dentition of a patient and/or color 2D images.

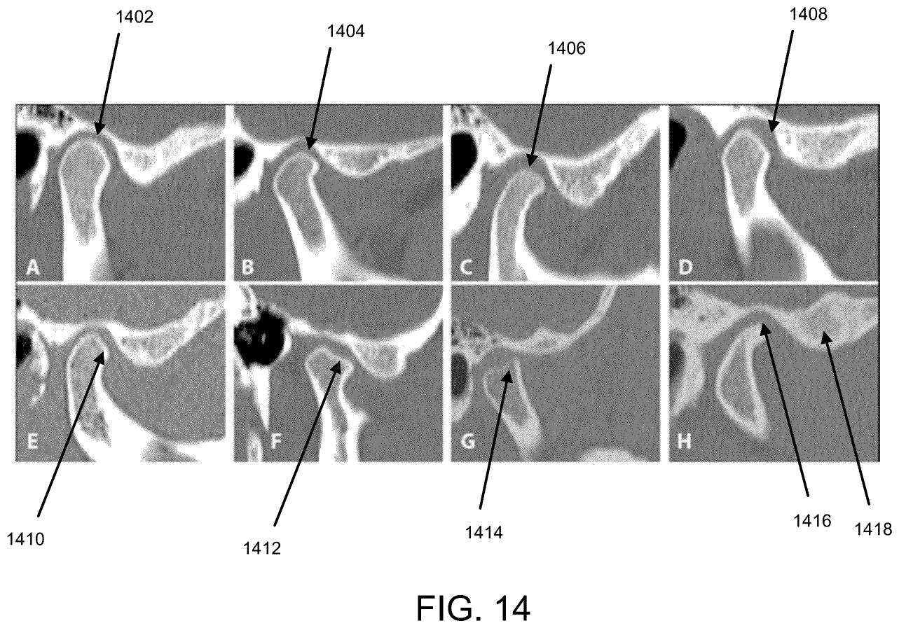

In some embodiments, some or all of the data may be labeled with segmentation information, TMD indicator information (e.g., indicating of osseous changes (e.g., as illustrated in FIGS. 14-16), audio-based indicators of TMD, image-based indicators of TMD, video-based indicators of TMD, etc.), and/or other information. The segmentation information may identify features such as mandible, teeth, the TMJ's cartilage disc, etc.

In some embodiments, some of the image-based data in training dataset 1310 can be processed by a segmenter 1315 that segments the image-based data into multiple different features (e.g., mandible, teeth, the TMJ's cartilage disc, etc.), and that outputs segmentation information 1318 for the image-based data. The segmenter 1315 may be or include, for example, a trained machine learning model such as a convolutional neural network (CNN) trained to classify pixels or regions of input images into different classes. This can include performing point-level classification (e.g., pixel-level classification or voxel-level classification) of different types of features and/or objects of subjects of images. The different features and/or objects may include, for example, mandible, teeth, the TMJ's cartilage disc, etc. The segmenter 1315 may output one or more masks, each of which may have a same resolution as an input image. The mask or masks may include a different identifier for each identified feature or object, and may assign the identifiers on a pixel-level or patch-level basis. In one embodiment, different masks are generated for one or more different classes of features and/or objects. In one embodiment, a single mask or map includes segmentation information for all identified classes of features and/or objects. Some types of features are location-specific features and are represented in one or more masks.

In some embodiments, the segmenter 1315 performs one or more image processing and/or computer vision techniques or operations to extract segmentation information from images. Such image processing and/or computer vision techniques may or may not include the use trained machine learning models. Accordingly, in some embodiments, segmenter 1315 does not include a machine learning model.

At block 1338, data from the training dataset 1310, and optionally segmentation information 1318, may be used to train one or more machine learning models to indicate a likelihood of TMD. The training dataset containing hundreds, thousands, tens of thousands, hundreds of thousands, or more data points can be used to form the training dataset 1310 and optionally including segmentation information 1318. In embodiments, up to millions of scan data and segmentation information are included in a training dataset.

Training may be performed by inputting one or more data points and optionally corresponding segmentation information into the machine learning model one at a time. The data that is input into the machine learning model may include a single layer or multiple layers. In some embodiments, a recurrent neural network (RNN) is used. In such an embodiment, a second layer may include a previous output of the machine learning model (which resulted from processing a previous input).

The machine learning model processes the input to generate an output. An artificial neural network includes an input layer that consists of values in a data point. The next layer is called a hidden layer, and nodes at the hidden layer each receive one or more of the input values. Each node contains parameters (e.g., weights) to apply to the input values. Each node therefore essentially inputs the input values into a multivariate function (e.g., a non-linear mathematical transformation) to produce an output value. A next layer may be another hidden layer or an output layer. In either case, the nodes at the next layer receive the output values from the nodes at the previous layer, and each node applies weights to those values and then generates its own output value. This may be performed at each layer. A final layer is the output layer, where there is one node for each class, prediction and/or output that the machine learning model can produce. For example, for an artificial neural network being trained to output gingival recession measurement and/or categorization for each tooth.

Processing logic may then compare the generated measurements and/or categorizations to the known condition and/or label that was included in the training data item. Processing logic determines an error based on the differences between the output probability map and/or label(s) and the provided probability map and/or label(s). Processing logic adjusts weights of one or more nodes in the machine learning model based on the error. An error term or delta may be determined for each node in the artificial neural network. Based on this error, the artificial neural network adjusts one or more of its parameters for one or more of its nodes (the weights for one or more inputs of a node). Parameters may be updated in a back propagation manner, such that nodes at a highest layer are updated first, followed by nodes at a next layer, and so on. An artificial neural network contains multiple layers of “neurons,” where each layer receives input values from neurons at a previous layer. The parameters for each neuron include weights associated with the values that are received from each of the neurons at a previous layer. Accordingly, adjusting the parameters may include adjusting the weights assigned to each of the inputs for one or more neurons at one or more layers in the artificial neural network.

Once the model parameters have been optimized, model validation may be performed to determine whether the model has improved and to determine a current accuracy of the model. After one or more rounds of training, processing logic may determine whether a stopping criterion has been met. A stopping criterion may be a target level of accuracy, a target number of processed data items from the training dataset, a target amount of change to parameters over one or more previous data points, a combination thereof and/or other criteria. In one embodiment, the stopping criteria is met when at least a minimum number of data points have been processed and at least a threshold accuracy is achieved. The threshold accuracy may be, for example, 70%, 80% or 90% accuracy. In one embodiment, the stopping criteria is met if accuracy of the machine learning model has stopped improving. If the stopping criterion has not been met, further training is performed. If the stopping criterion has been met, training may be complete. Once the machine learning model is trained, a reserved portion of the training dataset 1310 (and optionally segmentation information 1318) may be used to test the model. Testing the model can include performing unit tests, regression tests, and/or integration tests.

Once one or more trained ML models are generated, they may be stored in model storage 1345. Multiple ML models can be trained and used in combination. For example, model training workflow 1305 can train an audio-based ML model, a video-based ML model, and/or a CBCT-based ML model. Audio-based ML model can output a value indicating a likelihood of TMD in audio data. Video-based ML model can output a value indicating a likelihood of TMD in video data. CBCT-based ML model can output a value indicating a likelihood of TMD in CBCT scan data. In some embodiments, at block 1338, processing logic can train a single ML model that receives, as input, video, audio, and/or scan data, and can output a single value indicating a likelihood of TMD in the combination of data.

In some embodiments, model application workflow 1317 includes one or more trained machine learning models that function as audio-based ML model 1370, video-based ML model 1372, and/or CBCT-based ML model 1374. These logics may be implemented as separate machine learning models or as a single combined machine learning model, in embodiments. For example, segmentation ML model 1364, audio-based ML model 1370, video-based ML model 1372, and/or CBCT-based ML model 1374 may share one or more layers of a deep neural network. However, each of these logics may include distinct higher level layers of the deep neural network that are trained to generate different types of outputs.

In some embodiments, a patient, a dental professional (e.g., a doctor, dentist, hygienist, or technician), and/or another individual may capture an audio recording of the patient performing at least one of opening, closing, lateral, or protrusive jaw movements. In some embodiments, the audio recording may be from a standalone microphone, or a microphone built into a separate device (e.g., a mobile phone, a video camera, etc.). In some embodiments, a dental professional (e.g., doctor, dentist, hygienist, or technician) may capture an intraoral scan of a patient. The intraoral scanner may include a built-in microphone, which can record audio as the patient performing at least one of opening, closing, lateral, or protrusive jaw movements. The audio recording may correspond to audio data 348, and/or audio data 352 of FIG. 3. In some embodiments, the audio recording data can be preprocessed (e.g., by input preprocessing engine 312 of FIGS. 3,8) to filter out background noise, amplify the frequencies corresponding to the sound of TMD, dampen the frequencies corresponding to sounds not associated with TMD, and/or to generate a spectrogram of the audio signals.

In some embodiments, a patient, dental profession (e.g., a doctor, a dentist, a hygienist, or a technician), and/or another individual may capture a video recording of the patient performing at least one of opening, closing, lateral, or protrusive jaw movements. In some embodiments, the video recording can be preprocessed (e.g., by input preprocessing engine 312 of FIGS. 3,8) to stabilize the video, and/or to identify and segment individual frames of the video. In some embodiments, the video recordings may correspond to video data 1350, and/or video data 353 of FIG. 3. In some embodiments, the video frame data can be segmented by segmentation ML model 1364.

In some embodiments, a dental profession (e.g., a doctor, a dentist, a hygienist, or a technician) may capture CBCT scan(s) of a patient, e.g., with the patient's jaw in an open position and/or in a closed position. In some embodiments, the CBCT-scan data can be preprocessed (e.g., by input preprocessing engine 312 of FIGS. 3, 8) to segment the CBCT image data. In some embodiments, the CBCT scans may correspond to CBCT scan data 1352, and/or scan data 351 of FIG. 3. In some embodiments, the CBCT scan data can be segmented by segmentation ML model 1364.

The dental professional may have previously captured a CBCT scan, an audio recording, a video recording, and/or an intraoral scan of the patient, and/or may have other patient data, such as the patient's chart, the patient's previous TMD assessments and/or diagnoses, the patient's previous treatment of TMD (or of other medical ailments), the patient's answers to a questionnaire (optionally including a history of patient's answers), and/or the patient's occlusion data, which may correspond to patient data 1354. Audio data 1348, video data 1350, CBCT scan data 1352, and/or patient data 1354 may be combined to form input data 1362. Some or all of input data 1362 may be processed by segmentation ML model 1364. In some embodiments, segmentation ML model 1364 may perform the same functions as segmenter 1315. Segmentation ML model 1364 may produce output 1368, which can include segmentation information identifying mandible, skull, teeth, TMJ's cartilage disc, fossa, condyle, etc.

Input data 1362 and/or output 1368 can be provided as input to audio-based ML model 1370, video-based ML model 1372, and/or CBCT-based ML model 1375. Audio-based ML model 1370 may produce output 1371, which may include a value indicating a likelihood of the presence of TMD in an audio recording (and optionally including additional patient data 1354) of the patient. In some embodiments, audio-based ML model 1370 can include two (or more) ML models, one trained to indicate a likelihood of the presence of TMD in an intraoral audio recording, and one trained to indicate a likelihood of the presence of TMD in an audio recording taken from outside the patient's oral cavity. Video-based ML model 1372 may produce output 1373, which may include a value indicating a likelihood of the presence of TMD in a video recording (and optionally including additional patient data 1354) of the patient. CBCT-based ML model 1374 may produce output 1375, which may include a value indicating a likelihood of the presence of TMD in a CBCT-scan (and optionally including additional patient data 1354) of the patient. The value indicating the likelihood of the presence of TMD may be a value between 0 and 1 (inclusive), in which a higher value indicates a higher likelihood of TMD. Output aggregator 1376 may aggregate outputs 1371, 1373, 1375, and may optionally include additional patient data 1354, to produce aggregated output 1378. Thus, the model application workflow 1317 may produce, as aggregated output, a single value indicating a likelihood of the presence of TMD for a patient, based on audio data, video data, CBCT scan data, and/or patient data.

In some embodiments, treatment recommendation engine 325 of FIG. 3 can use the output 1371, 1373, 1375, and/or aggregated output 1378, to identify a potential cause of the TMD and/or to identify a treatment recommendation for the TMD. In some embodiments, report generation engine 330 of FIG. 3 can use the output 1371, 1373, 1375, and/or aggregated output 1378, combined with the treatment identified by treatment recommendation engine 325, to generate a TMD detection, assessment, and/or diagnosis report. The generated report can include, for example, the detection of TMD, the severity of the TMD, the frame and/or scan images (optionally overlaid with segmentation information of output 1368), and/or the treatment recommendation identified by treatment recommendation engine 325. The severity of the TMD can correspond to the output 1371, 1373, 1375, and/or aggregated output 1378. For example, the output 1371, 1373, 1375, and/or 1378 can include a value indicating a likelihood of the presence of TMD (e.g., a value between 0 and 1, where a higher value indicates a higher likelihood of the presence of TMD). The severity can correspond to the value. Thus, a value that exceeds a first threshold can indicate the presence of TMD, and a value that exceeds a second (higher) threshold can indicate a higher severity of the TMD.

FIG. 14 illustrates example sagittal views of CT images A-H of condyles representing examples of non-osteoarthritic or indeterminate osseous changes, in accordance with some embodiments of the present disclosure. Images A-B illustrate rounded condylar head 1402-1404, and well-defined cortical margin. Image C represents a rounded condylar head 1406, and well-defined noncortical margin. Images D-E are indeterminate for osteoarthritis, representing slight flatting of anterior slope and well-defined cortical margin 1408, 1410. Image F is indeterminate for osteoarthritis, representing flattening of anterior slope and a pointed anterior that is not sclerosed, well-defined cortical margin, and fossa that is shallow 1412. Image G represents a well-defined cortical margin that has a notch on the superior part 1414, illustrating a deviation in form, and fossa that is shallow. Image H represents narrowed appearance of the condylar head near medial part 1416, close position of the cortical plates giving the impression of sclerosis, and a non-osteoarthritic condyle.

In some embodiments, TMD detection/diagnostics engine 320 of FIG. 3 can provide one or more of images A-H as input to a machine learning model (e.g., model 1374 of FIG. 13) that is trained to output a likelihood of the presence of TMD. For example, the machine learning model can output a value indicating a likelihood of TMD for images G and H. In some embodiments, CBCT-based TMD detection engine 826 of FIG. 8 can identify images G and H as indicating a likelihood of the TMD.

In some embodiments, TMD detection/diagnostics engine 320 of FIG. 3 can segment one or more of image A-H to identify a first portion (e.g., the condylar head, e.g., labels 1402, 1404) and a second portion (e.g., fossa, e.g., label 1406). TMD detection/diagnostics engine 320 of FIG. 3 can compare the position of the first portion to the second portion to determine that the position is abnormal. The abnormal positioning can indicate a likelihood or presence of TMD.

In some embodiments, TMD detection/diagnostics engine 320 of FIG. 3 can segment one or more of image A-H to identify a bone density of a first portion (e.g., condyle, e.g., label 1412, 1414) and a bone density of a second portion (e.g., skull, e.g., label 1418). TMD detection/diagnostics engine 320 of FIG. 3 can compare the bone density of the first portion to the bone density of the second portion. If the difference between the two bone densities satisfies a criterion (e.g., is above a threshold value), TMD detection/diagnostics engine 320 of FIG. 2 can determine that TMD is likely present in the patient.

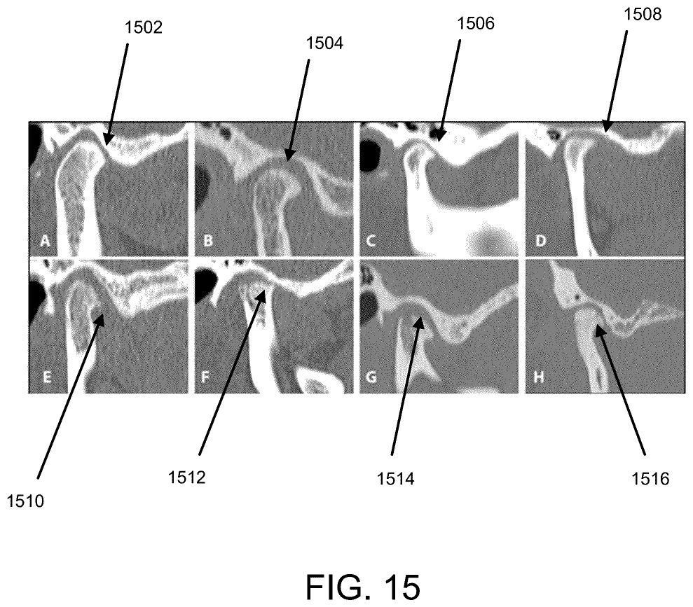

FIG. 15 illustrates examples of sagittal views of CT images A-H of condyles representing osseous changes, and corresponding osteoarthritis (OA) diagnoses, in accordance with some embodiments of the present disclosure. Image A is indeterminate for osteoarthritis (OA), illustrating subcortical sclerosis without any flattening, without erosion. Image B illustrates the presence of OA, displaying subcortical sclerosis, osteophytic growth on the anterior part of the condyle. Image C illustrates the presence of OA, displaying subcortical sclerosis, flattened posterior slope of the eminence, osteophytic growth on the anterior part of the condyle, limited joint space superiorly. Image D illustrates the presence of OA, displaying flattened superior margin, osteophytic growth at the anterior, fossa is shallow. Image E illustrates the presence of OA, displaying flattened posterior slope of the eminence, condylar margin is eroded and lacks corticated border, osteophytic growth. Image F illustrates the presence of OA, displaying flattened superior margin, decreased condylar height, margin is eroded and lacks corticated border, osteophytic growth, outline of the fossa is irregular. Image G illustrates the present of OA, displaying a bony cavity below the articular surface margin (i.e., subcortical cyst), osteophytic growth, posterior slope of the eminence is sclerosed. Image H illustrates the presence of OA, displaying generalized sclerosis, surface erosion, osteophytic growth, sclerosed fossa.

In some embodiments, TMD detection/diagnostics engine 320 of FIG. 3 can provide one or more of images A-H as input to a machine learning model (e.g., model 1374 of FIG. 13) that is trained to output a likelihood of the presence of TMD. For example, the machine learning model can output a value indicating a likelihood of TMD for images B through H. In some embodiments, CBCT-based TMD detection engine 826 of FIG. 8 can identify images B and H as indicating a likelihood of the TMD.

In some embodiments, TMD detection/diagnostics engine 320 of FIG. 3 can segment one or more of image A-H to identify a first portion (e.g., condylar head, e.g., label 1512, 1514) and a second portion (e.g., fossa, e.g., label 1504). TMD detection/diagnostics engine 320 of FIG. 3 can compare the position of the first portion to the second portion to determine that the position is abnormal. The abnormal positioning can indicate a likelihood or presence of TMD.

In some embodiments, TMD detection/diagnostics engine 320 of FIG. 3 can segment one or more of image A-H to identify a bone density of a first portion (e.g., condyle, e.g., label 1512) and a bone density of a second portion (e.g., skull, e.g., label 1506). TMD detection/diagnostics engine 320 of FIG. 3 can compare the bone density of the first portion to the bone density of the second portion. If the difference between the two bone densities satisfies a criterion (e.g., is above a threshold value), TMD detection/diagnostics engine 320 of FIG. 3 can determine that TMD is likely present in the patient.

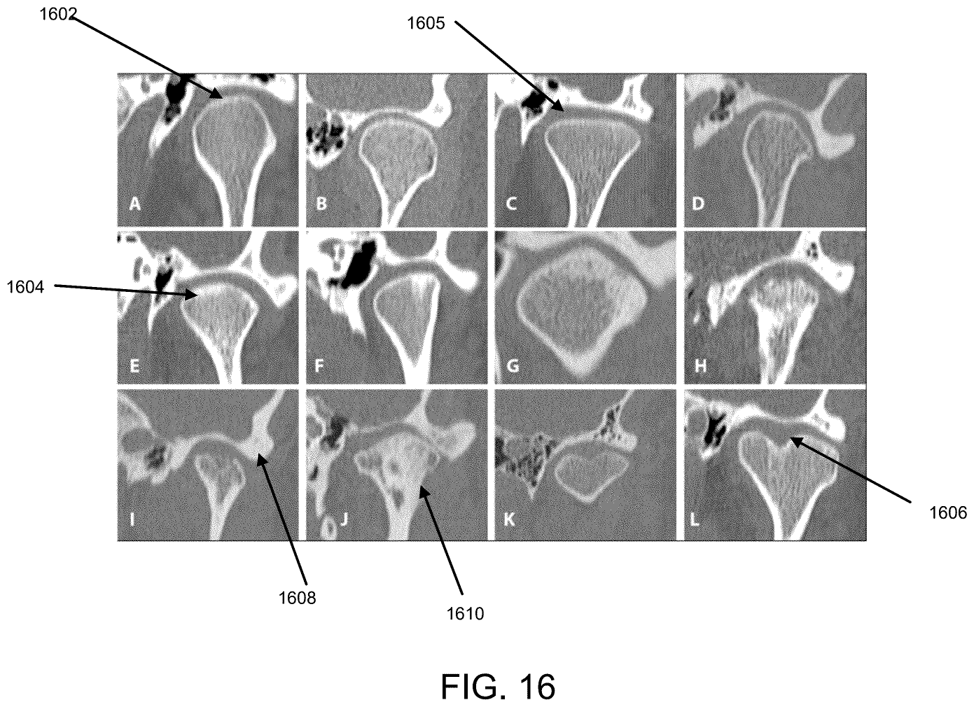

FIG. 16 illustrates example axially corrected coronal view of CT images A-L of condyles representing examples of osseous changes, and corresponding osteoarthritis (OA) diagnoses, in accordance with some embodiments of the present disclosure. Images A-B illustrate non-osteoarthritic condyles, displaying rounded condylar head, and well-defined cortical margin. Image C illustrates non-osteoarthritic condyle, displaying flattened superior margin, and well-defined cortical margin. Image D illustrates non-osteoarthritic condyle, displaying flattened lateral slope, and well-defined cortical margin. Image E is indeterminate for OA, displaying rounded condylar head and subcortical sclerosis. Image F is indeterminate for OA, displaying subcortical sclerosis. G. OA: subcortical sclerosis, surface erosion. Images H-I illustrate a presence of OA, displaying surface erosion. Image J illustrates a presence of OA, displaying generalized sclerosis, and subcortical cysts. Image K illustrates a non-osteoarthritic condyle, displaying well-defined corticated margin, bifid appearance, deviation in form. Image L illustrates a non-osteoarthritic condyle, displaying subcortical sclerosis in non-articulating surface, bifid appearance, deviation in form.

In some embodiments, TMD detection/diagnostics engine 320 of FIG. 3 can provide one or more of images A-L as input to a machine learning model (e.g., model 1374 of FIG. 13) that is trained to output a likelihood of the presence of TMD. For example, the machine learning model can output a value indicating a likelihood of TMD for images G-J. In some embodiments, the machine learning model can output a value indicating lesser likelihood of TMD for image F. In some embodiments, the likelihood of the presence of TMD in image F can be further refined by combining the output of the machine learning model with questionnaire answers from the patient and/or audio and/or video recordings of the patient performing at least one of opening, closing, lateral, or protrusive jaw movements. In some embodiments, CBCT-based TMD detection engine 826 of FIG. 8 can identify images G-J as indicating a likelihood of the TMD.

In some embodiments, TMD detection/diagnostics engine 320 of FIG. 3 can segment one or more of image A-H to identify a first portion (e.g., condylar head, e.g., label 1604) and a second portion (e.g., fossa, e.g., label 1605). TMD detection/diagnostics engine 320 of FIG. 3 can compare the position of the first portion to the second portion to determine that the position is abnormal. The abnormal positioning can indicate a likelihood or presence of TMD.

In some embodiments, TMD detection/diagnostics engine 320 of FIG. 3 can segment one or more of image A-H to identify a bone density of a first portion (e.g., condyle, e.g., label 1604) and a bone density of a second portion (e.g., skull, e.g., label 1608). TMD detection/diagnostics engine 320 of FIG. 3 can compare the bone density of the first portion to the bone density of the second portion. If the difference between the two bone densities satisfies a criterion (e.g., is above a threshold value), TMD detection/diagnostics engine 320 of FIG. 3 can determine that TMD is likely present in the patient. In some embodiments, TMD detection/diagnostics engine 320 of FIG. 3 can identify the degeneration of the condyle (e.g., condyle 1610) to identify a likelihood of TMD.

FIG. 17 illustrates a diagrammatic representation of a machine in the example form of a computing device 1700 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computing device 1700 corresponds to any computing device of FIGS. 1-3.

The example computing device 1700 includes a processing device 1702 (e.g., a CPU), a main memory 1704 (e.g., read-only memory (ROM), flash memory, dynamic random access memory (DRAM) such as synchronous DRAM (SDRAM), etc.), a static memory 1706 (e.g., flash memory, static random access memory (SRAM), etc.), and a secondary memory (e.g., a data storage device 1728), which communicate with each other via a bus 1708.

Processing device 1702 represents one or more general-purpose processors such as a microprocessor, central processing unit, or the like. More particularly, the processing device 1702 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1702 may also be one or more special-purpose processing devices such as an application specific integrated circuit (ASIC), a field programmable gate array (FPGA), a digital signal processor (DSP), network processor, or the like. In accordance with one or more aspects of the present disclosure, processing device 1702 is configured to execute the processing logic (instructions 1726, which may implement the dental diagnostics system 109 of FIG. 1) for performing operations and steps discussed herein. While only a single example processing device is illustrated, the term “processing device” shall also be taken to include any collection of processing devices (e.g., computers) that individually or jointly execute a set (or multiple sets) of instructions to perform any one or more of the methods discussed herein.

The computing device 1700 may further include a network interface device 1722 for communicating with a network 1764. The computing device 1700 also may include a video display unit 1710 (e.g., a liquid crystal display (LCD) or a cathode ray tube (CRT)), an alphanumeric input device 1712 (e.g., a keyboard), a cursor control device 1714 (e.g., a mouse, a touch-screen control device), and a signal generation device 1720 (e.g., a speaker).

The data storage device 1728 may include a machine-readable storage medium (or more specifically a non-transitory computer-readable storage medium) 1724 on which is stored one or more sets of instructions 1726 embodying any one or more of the methodologies or functions described herein. A non-transitory storage medium refers to a storage medium other than a carrier wave. The instructions 1726 may also reside, completely or at least partially, within the main memory 1704 and/or within the processing device 1702 during execution thereof by the computer device 1700, the main memory 1704 and the processing device 1702 also constituting computer-readable storage media.

The computer-readable storage medium 1724 may also be used to store a dental diagnostics system 109, which may correspond to the similarly named component of FIG. 7. The computer readable storage medium 1724 may also store a software library containing methods for a dental diagnostics system 109. While the computer-readable storage medium 1724 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

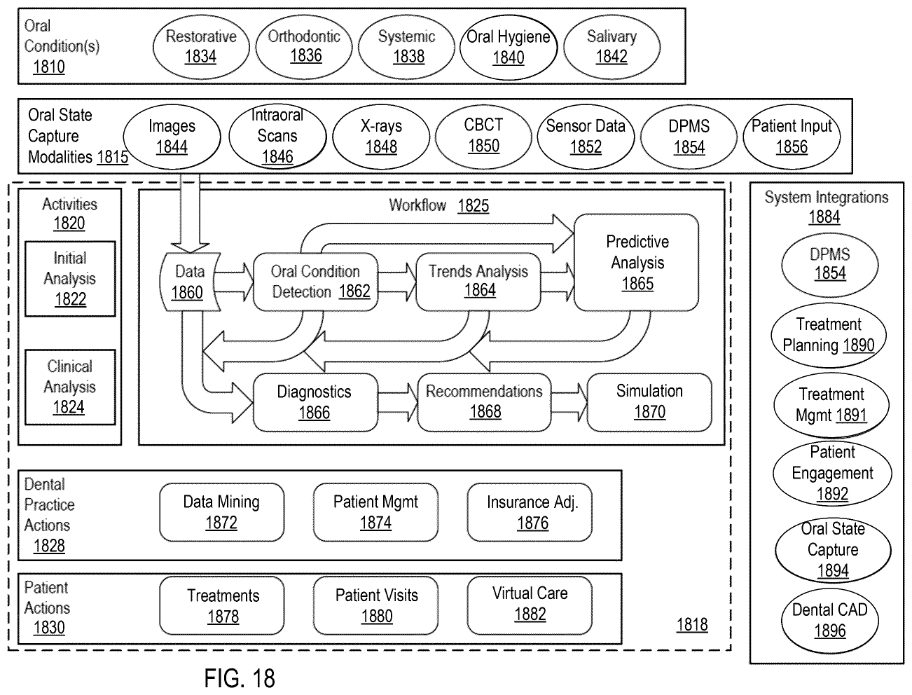

FIG. 18 illustrates a workflow 1825 for detecting, predicting, diagnosing and reporting on oral conditions (e.g., oral health conditions such as gingival recession, TMD, etc.) by an oral health diagnostics system 1818, in accordance with embodiments of the present disclosure. The workflow 1825 may be a general digital workflow covering use of radiographs and/or other oral state capture modalities within a digital platform of integrated products/services to provide identifications of oral conditions and/or actionable symptom recommendations and/or diagnoses of oral health problems associated with such oral conditions. The workflow 1825 may be used to assist doctors and/or users of an oral health diagnostics system 1818 to assess a patient's oral health, identify oral conditions, diagnose dental health problems, provide actionable symptom recommendations, provide treatment recommendations, and so on. For example, the workflow 1825 may be used to assist doctor's and/or users of an oral health diagnostics system 1818 to measure and/or categorize gingival recession, and/or to assess TMD, as described herein. The workflow 1825 may be executed by a digital platform of integrated products that provide dental condition identifications, actionable symptom recommendations, and/or diagnoses of oral health problems using analysis of data from one or more oral state capture modalities, including radiographs, CBCT scans, CT scans, and other 3D medical imaging modalities.

A patient may have one or more oral conditions 1810. Oral conditions 1810 may include or be related to caries, gingival recession, gingival swelling, tooth wear, bleeding, malocclusion, tooth crowding, tooth spacing, plaque, tooth stains, periodontitis, bone density loss, tooth cracks, and/or TMD, for example. In some embodiments, the oral conditions 1810 may include restorative conditions 1834, orthodontic conditions 1836, systematic conditions 1838, oral hygiene conditions 1840, salivary conditions 1842, and so on. Restorative conditions 1834 may include conditions such as caries that are addressable by performing restorative dental treatment. Such restorative dental treatment may include drilling and filling caries, performing root canals, forming preparations of teeth and applying caps or crowns to the preparations, pulling teeth, adding bridges to teeth, and so on. Restorative conditions may also include results of past restorative treatments of the patient's oral cavity. Examples of past restorations include fillings, caps, crowns, bridges, and so on. Orthodontic conditions 1836 may include conditions treatable via orthodontic treatment. Such orthodontic conditions may include a malocclusion (e.g., tooth crowding, overbite, underbite, posterior crossbite, posterior open bite, tooth gaps, etc.). Orthodontic conditions may be associated with restorative conditions in some instances. For example, tooth crowding may cause caries, which results in restorative treatment. Systematic conditions 1838 may include conditions such as periodontitis, periodontal bone loss, gingival recession, tooth wear, occlusal trauma within the mouth (e.g. a chip, a crack, a fracture, and/or wear of the tooth or restoration (e.g., a flattened surface, exposed dentin, etc.)), TMD, and so on. Systematic conditions 1838 may be associated with restorative conditions 1834 and/or orthodontic conditions 1836. Oral hygiene conditions 1840 may include brushing and flossing related conditions, such as development of calculus on teeth, caries, and so on. Oral hygiene conditions 1840 may be related to restorative conditions 1834, orthodontic conditions 1836 and/or systematic conditions 1838 in embodiments. Salivary conditions 1842 may include a pH level of a patient's mouth that is outside of normal, a low level of saliva, and so on. Salivary conditions 1842 may be related to restorative conditions 1834, orthodontic conditions 1836, systematic conditions 1838 and/or oral hygiene conditions 1840 in embodiments. For example, the detection and identification of salivary conditions may be used as an input to an ML model that can use such information to assess periodontal disease, acid reflux, vomiting, poor diet, oral cancer, and/or oropharyngeal cancer. For example, biomarkers of saliva may be used to assist in the assessment and/or management of periodontal disease. Tooth erosion, caries and/or saliva biomarkers may be used to identify acid reflux, vomiting and/or poor diet. In some instances, an oral condition of a patient may include a cross-classification. Such oral conditions may belong to multiple different categories of oral conditions 1810. For example, caries may be a restorative condition 1834, an orthodontic condition 1836, and an oral hygiene condition 1840.

A patient may have one or more oral health problems that may be root problems for the oral conditions and/or that may be caused by the oral conditions. In some embodiments, an oral condition also constitutes an oral health problem. Examples of oral health problems include gingival recession, TMD, caries, periodontal disease, a tooth root issue, a cracked tooth, a broken tooth, oral cancer, a cause of bad breath, and/or a cause of a malocclusion.

A dental practice (e.g., a group practice or solo practice) may capture data about a patient's oral state using one or more oral state capture modalities 1815. A common oral state capture modality used by dental practices are radiographs (i.e., x-rays) 1848. There are multiple different types of x-rays that a genal practice may capture of a patient's oral cavity, including bite-wing x-rays, panoramic x-rays and periapical x-rays.

A bite-wing x-ray is a type of dental radiograph used to detect dental caries (cavities) and monitor the health of teeth and supporting bone. During a bite-wing x-ray, the patient bites down on a small tab or wing-shaped device attached to the x-ray film or sensor. This helps keep the film or sensor in place while the x-ray is taken. An x-ray machine is positioned outside the mouth to capture images of the upper and lower teeth on one side of the mouth at a time. Accordingly, a bite-wing x-ray includes upper and lower teeth of one side of a patient's mouth. In embodiments, bite-wing x-rays are useful for detecting cavities between teeth and for assessing the fit of dental fillings and crowns. Bite-wing x-rays may also be used to help in diagnosing gum disease and/or to monitor bone levels around the teeth in embodiments.

A periapical x-ray, also known as a periapical radiograph, is a type of dental x-ray that focuses on specific areas of the mouth, particularly individual teeth and the surrounding bone. During a periapical x-ray, the dentist or dental radiographer positions an x-ray machine so that it captures detailed images of one or more teeth from crown to root, as well as the surrounding bone structure and supporting tissues. Periapical x-rays may provide a comprehensive view of the entire tooth, including the root tip (apex) and the bone around the tooth's root. In embodiments, periapical x-rays may be used to help diagnose oral health problems such as tooth decay (caries), infections or abscesses at the root of a tooth, bone loss around a tooth due to periodontal (gum) disease, abnormalities in the root structure or surrounding bone, evaluation of dental trauma or injuries, and so on. Periapical x-rays may also be used to assist in assessment of the status of teeth prior to dental procedures such as root canal treatment or extraction.

A panoramic x-ray, also known as a panoramic radiograph or orthopantomogram (OPG), is a type of dental radiograph that provides a comprehensive view of the entire mouth, including the teeth, jaws, temporomandibular joints (TMJ), and surrounding structures in a single image. During a panoramic x-ray, the patient stands or sits in an upright position while an x-ray machine rotates around their head in a semi-circle. The x-ray machine captures a continuous image as it moves, creating a detailed panoramic view of the entire oral and maxillofacial region. In embodiments, a panoramic x-ray may be used to assist in evaluation of the development and position of teeth, including impacted teeth, assessing the health of the jawbone and surrounding structures, detecting cysts, tumors, or other abnormalities in the jaw or adjacent tissues, planning orthodontic treatment by assessing tooth alignment and development, evaluating the placement and condition of dental implants, and/or diagnosing temporomandibular joint (TMJ) disorders or other jaw-related issues.

Another oral state capture modality that is increasingly common in dental practices are intraoral scans 1846, and three-dimensional (3D) models of dental arches (or portions thereof) based on such intraoral scans. Intraoral scans are produced by an intraoral scanning system that generally includes an intraoral scanner and a computing device connected to the intraoral scanner by a wired or wireless connection. The intraoral scanner is a handheld device equipped with one or more small cameras and/or optical sensors. The dentist or dental professional moves the intraoral scanner around the patient's mouth, capturing multiple 3D images or scans of the teeth and surrounding structures from various angles. As the intraoral scanner captures the images or scans, they may be processed and displayed on a computer screen in real-time or near real-time. The collected images or scans are stitched together to create a complete 3D digital model of the patient's teeth and oral cavity. This digital impression can be manipulated, analyzed, and shared electronically with dental laboratories or specialists as needed.

An intraoral scan application executing on the computing device of an intraoral scanning system may generate a 3D model (e.g., a virtual 3D model) of the upper and/or lower dental arches of the patient from received intraoral scan data (e.g., images/scans). To generate the 3D model(s) of the dental arches, the intraoral scan application may register and stitch together the intraoral scans generated from an intraoral scan session. In one embodiment, performing image registration includes capturing 3D data of various points of a surface in multiple intraoral scans, and registering the intraoral scans by computing transformations between the intraoral scans. The intraoral scans may then be integrated into a common reference frame by applying appropriate transformations to points of each registered intraoral scan.

In one embodiment, registration is performed for each pair of adjacent or overlapping intraoral scans. Registration algorithms may be carried out to register two adjacent intraoral scans for example, which essentially involves determination of the transformations which align one intraoral scan with the other. Registration may involve identifying multiple points in each intraoral scan (e.g., point clouds) of a pair of intraoral scans, surface fitting to the points of each intraoral scans, and using local searches around points to match points of the two adjacent intraoral scans. For example, the intraoral scan application may match points, edges, curvature features, spin-point features, etc. of one intraoral scan with the closest points, edges, curvature features, spin-point features, etc. interpolated on the surface of the other intraoral scan, and iteratively minimize the distance between matched points. Registration may be repeated for each adjacent and/or overlapping scans to obtain transformations (e.g., rotations around one to three axes and translations within one to three planes) to a common reference frame. Using the determined transformations, the intraoral scan application may integrate the multiple intraoral scans into a first 3D model of the lower dental arch and a second 3D model of the upper dental arch.

The intraoral scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

Oral state capture modalities 1815 may additionally or alternatively include one or more types of images 1844 (e.g., 2D and/or 3D images) of a patient's oral cavity. In addition to generating intraoral scans, intraoral scanning systems may additionally be used to generate color 2D images of a patient's oral cavity. These color 2D images may be registered to the intraoral scans generated by the intraoral scanning system, and may be used to add color information to 3D models of a patient's dental arches. Intraoral scanning systems may additionally or alternatively generate 2D near infrared (NIR) images, images generated using fluorescent imaging, images generated under particular wavelengths of light, and so on. Such image generation may be interleaved with 3D image or intraoral scan generation by an intraoral scanner.

Dental practices may additionally include cameras for generating 3D images of a patient's oral cavity and/or cameras for generating 2D images of a patient's oral cavity. Additionally, a patient may generate images of their own oral cavity using personal cameras, mobile devices (e.g., tablet computers or mobile phones), and so on. In some instances, patients may generate images of their oral cavity based on the instruction of an application or service such as a virtual dental care application or service. In some cases, images of a patient's oral cavity (e.g., those taken by a dental practitioner or by a patient themselves) may be taken while the patient wears a cheek retractor to retract the lips and cheeks of the patient and provide better access for dental imaging (i.e., for intraoral photography).

Some dental practices also use cone beam computed tomography (CBCT) 1850 as an oral state capture modality 1815. CBCT is a medical imaging technique that uses a cone-shaped X-ray beam to create detailed 3D images of the dental and maxillofacial structures. CBCT scanners may be specifically designed for imaging the head and neck region, including the teeth, jawbones, facial bones, and surrounding tissues. A CBCT machine emits a cone-shaped X-ray beam that rotates around the patient's head. A detector on the opposite side of the machine captures a sequence of X-ray images from different angles. The x-ray images are processed to reconstruct them into a detailed 3D volumetric dataset. This dataset provides a comprehensive view of the patient's oral anatomy in three dimensions. CBCT scans may facilitate accurate diagnosis of various dental and maxillofacial conditions, including impacted teeth, dental infections, bone abnormalities, and temporomandibular joint disorders. In embodiments, CBCT imaging may be used for various dental and maxillofacial applications, including implant planning, orthodontic treatment planning, endodontic evaluations, oral surgery, and periodontal assessments. In embodiments, the output of a CBCT scan consists of a series of grayscale cross-sectional images that can be reconstructed into 3D models for detailed analysis of bone structures, teeth, airways, and soft tissues. CBCT scans can be displayed in different planes, including an axial (horizontal) plane (including slices from top to bottom), a sagittal (side view) plane (including slices from left to right), and a coronal (front view) plane (including slices from front to back). Additionally, CBCT scans may be output as a 3D volume rendering, providing a complete 3D representation of a scanned area (e.g., a patient's mouth, dentition, jaw, etc. The final CBCT scan data may be stored in the DICOM (Digital Imaging and Communications in Medicine) format in some embodiments, enabling radiologists, dentists, and specialists to analyze them using advanced imaging software.

Other types of oral state capture modalities 1815 that may be used to collect medical data about a patient's dentition is a CT scan and a magnetic resonance imaging (MRI) scan. MRI is a non-invasive medical imaging technique that uses strong magnetic fields and radio waves to generate detailed images of the internal structures of the body. It is particularly useful for visualizing soft tissues, such as the brain, muscles, and organs, without using ionizing radiation (like X-rays or CT scans). MRI works by aligning hydrogen atoms in the body with a magnetic field and then using radiofrequency pulses to detect their signals, which are processed into high-resolution images. The output of an MRI is a set of high-resolution cross-sectional images or 3D reconstructions of the body's internal structures. These images are typically in grayscale, where different shades represent various tissue densities and compositions. MRI scans can be displayed in different planes, including an axial (horizontal) plane (including slices from top to bottom), a sagittal (side view) plane (including slices from left to right), and a coronal (front view) plane (including slices from front to back). In embodiments, the final MRI output may be in the DICOM format, which allows medical professionals to analyze and interpret the images using specialized software.

Computed Tomography (CT) is a medical imaging technique that uses X-rays and computer processing to create detailed cross-sectional images of the body's internal structures. It is particularly useful for visualizing bones, blood vessels, and soft tissues, making it valuable in diagnosing injuries, tumors, and internal bleeding. The output of a CT scan consists of a series of grayscale cross-sectional images that represent different tissue densities. These images can be reconstructed into 3D models for better visualization. CT scans can be displayed in different planes, including an axial (horizontal) plane (including slices from top to bottom), a sagittal (side view) plane (including slices from left to right), and a coronal (front view) plane (including slices from front to back). In embodiments, the final CT output may be in the DICOM format.

For image-based oral state capture modalities, multiple depictions and views of the oral cavity and internal structures can be captured (e.g., in radiographs, intraoral scans, etc.). Examples of views include occlusal views, buccal views, lingual views, proximal-distal views, panoramic views, periapical views, bitewings views, and so on. Additionally, for 3D image-based oral state capture modalities, the 3D image data may be output as a 3D surface, a 3D volume, a series of 2D slices in one or more planes (e.g., sagittal, coronal, axial, etc.), and so on.

Oral state capture modalities 1815 may additionally or alternatively include sensor data 1852 from one or more worn sensors. In some instances, a patient may be prescribed a compliance device (e.g., an electronic compliance indicator), an orthodontic aligner, a palatal expander, a sleep apnea device, a night guard, a retainer, or other dental appliance to be worn by the patient. Any such dental appliance may include one or more integrated sensors, which may include force sensors, pressure sensors, pH sensors, sensors for measuring saliva bacterial content, temperature sensors, contact sensors, bio sensors, and so on. Sensor data from the sensor(s) of a dental appliance worn by a patient may be reported to oral health diagnostics system 1818 in embodiments. Additionally, or alternatively, a patient may wear one or more consumer health monitoring tools or fitness tracking devices, such as a watch, ring, etc. that includes sensors for tracking patient activity, heartbeat, blood pressure, electrical heart activity (e.g., generates an electrocardiogram), breathing, sleep patterns, body temperature, and so on. Data collected by such fitness tracking devices may also be reported to the oral health diagnostics system 1818 in embodiments.

Oral state capture modalities 1815 may additionally or alternatively include patient input 1856. Patient input may include patient complaints of pain, numbness, bleeding, swelling, clicking, etc. in one more regions of their mouth. Patient input may further include input on overall health, such as information on underlying health conditions (e.g., diabetes, high blood pressure, etc.), on patient age, and so on. Such patient input may be captured and input into an oral health diagnostics system 1818 in embodiments. For example, a doctor or patient may type up notes or annotations indicating the patient input, which may be ingested by the oral health diagnostics system 1818 with other oral state capture modalities 1815.

In some embodiments, an oral health diagnostics system 1818 may include one or more system integrations 1884 with external systems, which may or may not be dental related. Such system integrations 1884 may be for data to be provided to the oral health diagnostics system 1818 and/or for the oral health diagnostics system 1818 to provide data to the other system(s).

Dental practices generally use a dental practice management system (DPMS) 1854 for managing the dental practices. A DPMS 1854 is a software solution designed to streamline and automate various administrative and clinical tasks within a dental practice. DPMS 1854 are tailored for the needs of dental offices and help dentists and their staff manage patient information, appointments, billing, and other aspects of dental practice management efficiently. A DPMS 1854 allows a dental practice to maintain comprehensive patient records, including demographic information, medical history, treatment plans, and clinical notes. The DPMS 1854 provides a centralized database that enables dental staff to access patient information quickly and efficiently. DPMS 1854 generally includes features for scheduling patient appointments, managing appointment calendars, and sending appointment reminders to patients. DPMS 1854 provides tools for creating and managing treatment plans for patients, including digital charting of dental procedures, diagnoses, and treatment progress. This helps dentists and hygienists track patient care effectively and ensure continuity of treatment. DPMS 1854 may help to automate billing processes, including generating invoices, processing payments, and managing insurance claims. It can also verify patient insurance coverage, estimate treatment costs, and submit claims electronically to insurance providers for faster reimbursement. DPMS 1854 may generate financial reports and analytics to help dental practices track revenue, expenses, and profitability.

In embodiments, data from a DPMS 1854 is used as one type of oral state capture modality 1815. Oral health diagnostics system 1818 may interface with a DPMS 1854 to retrieve patient records for a patient, including past oral conditions of the patient, doctor notes, patient information (e.g., name, gender, age, address, etc.), and so on.

In addition to an ability to ingest data from a DPMS 1854, oral health diagnostics system 1818 in embodiments may be able to generate reports and/or other outputs that can be ingested by the DPMS 1854. Accordingly, once the oral health diagnostics system 1818 performs an assessment of a patient's oral conditions, oral health problems, treatment recommendations, etc., the oral health diagnostics system 1818 may format such data into a format that can be understood by the DPMS 1854. The oral health diagnostics system may then automatically add new data entries to the DPMS 1854 for a patient based on an analysis of patient data from one or more oral state capture modalities 1815.

As previously mentioned, the oral health diagnostics system 1818 may have a system integration with one or more oral state capture systems (e.g., such as an intraoral scanner or intraoral scanning system, CBCT system, CT system, MRI system, etc.) 1894, from which intraoral scans 1846, images 1844, 3D models, 3D volumes, and/or data from one or more oral state capture modalities may be received.

In embodiments, an output of oral health diagnostics system 1818 may be provided to a dental computer aided drafting (CAD) system 1896, such as Exocad® by Align Technology. The dental CAD system 1896 may be used for designing dental restorations such as crowns, bridges, inlays, onlays, veneers, and dental implant restorations. The dental CAD system 1896 may provide a comprehensive suite of tools and features that enable dental professionals to create precise and customized dental restorations digitally. The dental CAD system 1896 may import digital impressions (e.g., 3D digital models of a patient's dental arches) captured using intraoral scanners, and may further import data on a patient's oral health from oral health diagnostics system 1818. For example, the oral health diagnostics system 1818 may export a report on a patient's oral health to the dental CAD system 1896, which may be used together with a digital impression of the patient's dental arches to develop an appropriate restoration for the patient, for implant planning, for planning of surgery for implant placement, and so on.

In embodiments, oral health diagnostics system 1818 may have a system integration 1884 with a patient engagement system 1892 (e.g., which may include a patient portal and/or patient application). The patient portal may be a portal to an online patient-oriented service. Similarly, the patient application may be an application (e.g., on a patient's mobile device, tablet computer, laptop computer, desktop computer, etc.) that interfaces with a patient-oriented service.

In an example, oral health diagnostics system 1818 may integrate with a virtual care system. The virtual care system may provide a suite of digital tools and services designed to enhance patient care and communication between orthodontists/dentists and their patients. The virtual care system may leverage technology to facilitate remote monitoring, consultation, and treatment planning, allowing patients to receive dental care more conveniently and effectively.

In one embodiment, the patient engagement system 1892 is or includes a virtual care system that may provide remote monitoring, teleconsultation, treatment planning, patient education and engagement, data management, and data analytics. With respect to remote monitoring, the virtual care system enables orthodontists and dentists to remotely monitor their patients' treatment progress (e.g., for orthodontic treatment) using advanced digital tools. This may include the use of smartphone apps, patient portals, or other software platforms that allow patients to capture and upload photos or videos of their teeth and orthodontic appliances. Such patient uploaded data may be provided to oral health diagnostics system 1818 for automated assessment in embodiments. With regards to patient education and engagement, the virtual care system may provide reports, presentations, etc. generated by oral health diagnostics system 1818 to patients (e.g., via a patient portal and/or application). For example, the oral health diagnostics system 1818 may automatically generate informational videos, treatment progress trackers, compliance reminders, reports, presentations, and so on that are tailored to a patient's oral health, which may be provided to the patient via the patient portal and/or application.

In embodiments, oral health diagnostics system 1818 may have a system integration 1884 with one or more treatment planning system 1890 and/or treatment management system 1891 such as ClinCheck® provided by Align Technology®. For example, oral health diagnostics system 1818 may have a system integration with an orthodontic treatment planning system and/or with a restorative dental treatment planning system. A treatment planning system 1890 may use digital impressions and/or a report output by oral health diagnostics system 1818 to plan an orthodontic treatment and/or a restorative treatment (e.g., to plan an ortho-restorative treatment). The treatment planning system 1890 may plan and simulate orthodontic and/or restorative treatments. Treatment management system 1891 may then receive data during treatment and determine updates to the treatment based on the treatment plan and the updated data.

In an example, an orthodontic treatment planning system may use advanced 3D imaging technology to create virtual models of patients' teeth and jaws based on digital impressions or intraoral scans. These digital models may be used to plan and simulate the entire course of orthodontic treatment, including the movement of individual teeth and the progression of treatment over time. Orthodontists can specify the desired tooth movements, treatment duration, and other parameters, taking into account a report provided by oral health diagnostics system 1818, to create personalized treatment plans tailored to each patient's unique anatomy, oral health, and preferences. The orthodontic treatment planning system enables orthodontists to simulate the step-by-step progression of orthodontic treatment virtually, showing patients how their teeth will gradually move and align over the course of treatment. Orthodontists can visualize the planned tooth movements in 3D and make adjustments as needed to optimize treatment outcomes. The orthodontic treatment planning system may provide orthodontists and patients with visualizations of the predicted treatment outcomes, including before-and-after simulations that demonstrate the expected changes in tooth position and alignment, and how those changes might affect the patient's overall oral health as optionally predicted by the oral health diagnostics system 1818. These visualizations help patients understand the proposed treatment plan and make informed decisions about their orthodontic care.

During treatment, updated data may be gathered about a patient's dentition, and such data (e.g., in the form of one or more oral state capture modalities 1815) may be processed by the oral health diagnostics system 1818, optionally in view of an already generated orthodontic treatment plan, to generate an updated report of the patient's overall oral health. The updated report may be provided by the oral health diagnostics system 1818 to the orthodontic treatment planning system and/or orthodontic treatment management system to enable the orthodontic treatment planning/management system to perform informed modifications to the treatment plan. Thus, integration of the oral health diagnostics system with the orthodontic treatment planning system and/or treatment management system supports an iterative design process, allowing orthodontists to review and refine treatment plans based on patient feedback, clinical considerations, treatment progress, and automated reports output by oral health diagnostics system 1818. This enables orthodontists to make adjustments to the treatment plan within the orthodontic treatment planning system and/or treatment management system and generate updated simulations to assess the impact of these changes on the final treatment outcome.

Accordingly, oral health diagnostics system 1818 may perform treatment planning and/or management on its own and/or based on integration with one or more treatment planning systems for planning and/or managing orthodontic treatment, restorative treatment, and/or ortho-restorative treatment. An output of such planning may be an orthodontic treatment plan, a restorative treatment plan, and/or an ortho-restorative treatment plan. A doctor may provide one or more modifications to the generated treatment plan, and the treatment plan may be updated based on the doctor modifications.

In addition to those systems mentioned herein that oral health diagnostics system 1818 may integrate with, oral health diagnostics system 1818 may integrate with any system, application, etc. related to dentistry and/or orthodontics.

Oral health diagnostics system 1818 may execute a workflow 1825 that includes processing and analysis of data 1860 from one or more oral state capture modalities 1815. The workflow 1825 may be roughly divided into activities 1820 associated with an initial analysis 1822 of a patient's oral health and operations associated with a clinical analysis 1824 of the patient's oral health in some embodiments. One of more of the operations of the workflow may be performed by and/or assisted by application of artificial intelligence and/or machine learning models in embodiments. Multiple embodiments are discussed with reference to machine learning models herein. It should be understood that such embodiments may also implement other artificial intelligence systems or models, such as large language models in addition to or instead of traditional machine learning models such as artificial neural networks.

The workflow may include performing oral condition detection at block 1862. To perform oral condition detection, one or more AI models may process the data 1860 to segment the data into one or more teeth, bones, tissue, ligaments, muscles, etc. and into one or more oral conditions that may be associated with the one or more of the teeth, bones, tissues, ligaments, muscles, etc. The one or more AI models and/or additional logic may operate on the data and/or on outputs of other trained machine learning models and/or logic to identify specific teeth and apply tooth numbering to the teeth, identify bones, identify tooth roots, identify soft tissues, identify oral conditions, associate the oral conditions to specific teeth, determine locations on the teeth at which the oral conditions are identified, and so on. Examples of anatomical (e.g., oral) structures and conditions that may be segmented include tooth and Root Issues (e.g., impacted teeth, root fractures, and resorption), caries (e.g., early-stage cavities and decay), periodontal Disease (e.g., bone loss and gum disease evaluation), temporomandibular joint (TMJ) disorders, endodontic assessment (e.g., root canal anatomy, infections, and cysts), jaw alignment issues, bone structure, tooth positioning, and so on. Other examples of detectable issues that may be segmented include facial and jaw fractures, cysts and tumors, anatomical variations, sinusitis, nasal obstructions, polyps, an airway (e.g., for airway analysis such as sleep apnea diagnosis), cranial abnormalities, and so on.

The output of block 1862 may include masks indicating pixels of input image data (e.g., radiographs, CBCT scans, 3D volumes, 3D models, 2D images, 2D slices of 3D volumes/surfaces/models, etc.) associated with particular dental conditions, indications of which teeth have detected oral conditions, masks indicating, for each tooth in the input data, which pixels represent that tooth, and so on. In some embodiments, oral condition detection 1862 includes dividing 3D image data into a temporal sequence of 2D images (e.g., 2D slices), and processing of the temporal sequence of 2D images to perform segmentation thereof, and outputting segmentation information of the temporal sequence of 2D images. Oral condition detection 1862 may additionally include adding the segmentation information of the temporal sequence of 2D images into the 3D image data that was divided into the temporal sequence of 2D images to generate 3D segmentation information. In embodiments, oral condition detection 1862 includes performing the operations described in one or more of the figures herein. The output of block 1862 may be input into one or more of block 1864, block 1865 and/or block 1866 in embodiments.

At block 1864, trends analysis may be performed based on the output of block 1862 and on prior oral conditions of the patient detected at one or more previous times. Trends analysis may include comparing oral conditions at one or more previous times to current oral conditions of the patient. Based on the comparison, an amount of change of one or more of the oral conditions may be determined, a rate of change of the one or more oral conditions may be determined, and so on. Trends analysis may be performed using traditional image processing and image comparison. Additionally, or alternatively, trends analysis may be performed by inputting current and past oral conditions and/or data from one or more oral state capture modalities into one or more trained machine learning models. An output of block 1864 may be provided to block 1865 and/or block 1866 in embodiments.

At block 1865, predictive analysis may be performed on the output of block 1862, on the output of block 1864 and/or on prior oral conditions of the patient detected at one or more previous times. Predictive analysis may include predicting future oral conditions of the patient based on input data. Predictive analysis may be performed with or without an input of prior oral conditions. If prior oral conditions are used in addition to current oral conditions to predict future conditions, then the accuracy of the prediction may be increased in embodiments. In some embodiments, predictive analysis is performed by projecting identified trends determined from the trends analysis into the future. In some embodiments, predictive analysis is performed by inputting the current and/or past oral conditions into one or more trained machine learning models that output predictions of future dental conditions. Predictive analysis may be performed using traditional image processing and image comparison. Additionally, or alternatively, predictive analysis may be performed by inputting current and/or past oral conditions, trends and/or data from one or more oral state capture modalities into one or more trained machine learning models. In embodiments, the predictive analysis generates synthetic image data, which may include panoramic views, periapical views, bitewing views, buccal views, lingual views, occlusal views, and so on of the predicted future oral conditions. Generated synthetic image data may be in the form of synthetic radiographs, synthetic color images, synthetic 3D models, and so on. An output of block 1865 may be provided to block 1866 in embodiments.

At block 1866, automated diagnostics of a patient's oral health may be performed based on data 1860 and/or based on outputs of block 1862, block 1864 and/or block 1865 in embodiments. In embodiments, one or more trained machine learning (ML) models and/or artificial intelligence (AI) models may process input data to perform the diagnostics. An output of the ML models and/or AI models may include actionable symptom recommendations usable to diagnose oral health problems and/or actual diagnoses of oral health problems associate with the detected oral conditions.

At block 1868, based on the data 1860, oral conditions identified at block 1862, output of trends analysis performed at block 1864, output of predictive analysis performed at block 1865 and/or output of diagnostics performed at block 1866, processing logic may generate one or more treatment recommendations for a patient. The treatment recommendations may include multiple different treatment options, with different probabilities of success associated with the different treatment options.

At block 1870, processing logic may generate one or more treatment simulations based on one or more of the treatment recommendations. The treatment simulations may include an alternative predictive analysis that shows predicted states of oral conditions and/or oral health problems of the patient after treatment is performed, or after one or more stages of treatment are performed. Treatment simulations may include generated synthetic image data, which may be in the form of synthetic radiographs, synthetic color images, synthetic 3D models, synthetic 3D volumes, synthetic 2D images, synthetic CBCT scans, synthetic CT scans, synthetic MRI scans, and so on. The synthetic image data may show what a patient's oral cavity would look like after treatment and/or after one or more intermediate and/or final stages of a multi-stage treatment (e.g., such as orthodontic treatment or ortho-restorative treatment).

Post treatment simulations may be compared to predicted simulations of the predicted states of the oral conditions absent treatment (e.g., as determined at block 1865) in embodiments.

In embodiments, a report may be generated including the data 1860 and/or outputs of one or more of blocks 1862, 1864, 1865, 1866, 1868 and/or 1870. The report may include labeled 2D and/or 3D images, labeled 3D volumes, labeled scans, labeled 3D surfaces, a dental chart, notes, annotations, and/or other information. The report may include a dynamic presentation (e.g., a video) that shows progression of dental conditions over time in some embodiments. The report may be stored and/or exported to one or more other systems (e.g., DPMS 1854, treatment planning system 1890, patient engagement system 1892, dental CAD system 1896).

The oral health diagnostics system 1818 may perform multiple dental practice actions 1828 and/or patient actions 1830 in addition to, or instead of, storing a generated report and/or exporting the report to other systems. Examples of dental practice actions 1828 that may be performed include data mining 1872, patient management 1874 and/or insurance adjudication 1876. Examples of patient actions 1830 that may be performed include treatments 1878, patient visits 1880 and/or virtual care 1882. One or more of the actions may be performed based on leveraging external systems in embodiments. For example, virtual care 1882 may be performed based on leveraging a patient portal and/or application of a virtual dental care system. Patient visits 1880 may be performed based on leveraging a DPMS 1854. Treatments 1878 may be performed based on leveraging a treatment planning system 1890 for planning, tracking and/or management of a treatment. Patient management 1874 and/or insurance adjudication 1876 may be performed based on leverage of a DPMS 1854.

Data mining 1872 may include analysis of patient data of a dental practice in embodiments. Data mining may be performed for a single dental practice or for multiple different dental practices. Data mining may be performed to determine strengths and weaknesses of a dental practice relative to other dental practices and/or to determine strengths and weaknesses of individual doctors relative to other doctors within a dental practice and/or outside of a dental practice (e.g., in a geographic region). As a result of data mining 1872, a report may be generated indicating things for a doctor to focus on, types of procedures that a doctor should perform more, oral state capture modalities that a doctor should use more frequently, and so on.

Patient management 1874 for a dental practice may include a range of tasks and processes aimed at providing quality care and ensuring positive experiences for patients throughout their interactions with the dental practice. Patient management may include appointment scheduling, patient registration and check-in, medical and dental history and records management (e.g., including information about past treatments, allergies, medications, and relevant medical conditions for each patient), treatment planning and coordination, financial management and billing (e.g., including collecting payments, processing insurance claims, providing cost estimates, and discussing payment options or financing arrangements with patients), patient communication and education (e.g., providing information about treatments, procedures, and oral hygiene instructions, as well as addressing patient concerns, answering questions, and maintaining open lines of communication throughout the treatment process), follow-up and recall, and patient satisfaction and feedback management.

Insurance adjudication 1876 for a dental practice refers to the process of evaluating and determining the coverage and reimbursement for dental services provided to patients by their dental insurance carriers. Insurance adjudication 1876 involves submitting claims to insurance companies, reviewing the claims for accuracy and completeness, and processing them according to the terms of the patient's insurance policy. After providing dental services (e.g., treatment) to a patient, the dental practice submits a claim to the patient's insurance company electronically or via paper. The claim includes information such as the patient's demographic details, treatment provided, diagnosis codes, procedure codes (CPT or ADA codes), and any other relevant documentation. In embodiments, such documentation is automatically prepared by oral health diagnostics system 1818. Upon receiving an insurance claim, the insurance company reviews the claim to determine coverage eligibility and benefits according to the terms of the patient's insurance policy. The insurance company evaluates the claim and calculates the amount of coverage and reimbursement based on the patient's benefits plan, contractual agreements with the dental office, and applicable fee schedules. The adjudication process may involve verifying the accuracy of the submitted information, applying deductibles, copayments, and coinsurance, and determining the allowed amount for each covered service. In embodiments, oral health diagnostics system 1818 may automatically generate responses to inquiries from insurance companies about already submitted claims. After adjudicating a claim, the insurance company sends an Explanation of Benefits (EOB) to the dental office and the patient. The EOB outlines the details of the claim, including the services rendered, the amount covered by insurance, any patient responsibility (such as copayments or deductibles), and the reason for any denials or adjustments. If the claim is approved, the insurance company issues payment to the dental office for the covered services. The dental office then reconciles the payment received with the treatment provided and updates the patient's financial records accordingly. If there are any discrepancies or denials, the dental office may need to follow up with the insurance company to resolve issues or appeal denied claims. In embodiments, oral health diagnostics system 1818 automatically handles such follow-ups. After insurance adjudication, the dental office bills the patient for any remaining balance or patient responsibility not covered by insurance, such as deductibles, copayments, or non-covered services. The patient is responsible for paying these amounts according to the terms of their insurance policy and the dental office's financial policies.

In some embodiments, the workflow 1825 can be implemented with just a few clicks of a web portal or dental practice application to enable doctors to purchase and activate one or more oral health diagnostics services. When patient records (e.g., data from one or more oral state capture modalities 1815, such as intraoral scans 1846, virtual care images 1844, digital x-rays 1848, CBCT scans 1850, etc.) are collected as a routine part of a dental appointment, these records may be uploaded to a digital platform of the oral health diagnostics system 1818. The oral health diagnostics system 1818 may start an analysis for the different oral (e.g., clinical) conditions that have been activated for the patient by the doctor, and may generate a report on the different identified oral conditions. In seconds the doctor may receive a report that has visual indications with colored clues of assessments for a number of possible dental conditions, dental health problems, and so on. As an example, the oral health diagnostics system 1818 can send this data to the treatment planning system 1890 or treatment management system 1891 to process.

In some embodiments, the treatment planning system 1890 can integrate this information with an orthodontic treatment plan. The doctor can share the analysis visually chairside with the patient and provide treatment recommendations based on the diagnosis. This can occur on the treatment planning and/or management system 1890, 1891 or on an application on an intraoral scanning system, CBCT system, MRI system or x-ray system, for example. The doctor can also share the analysis with the patient and send visual assessments via patient engagement system 1892. Integrated education modules may provide interactive context sensitive education tools designed to help the doctor diagnose and help convert the patient to the treatment in embodiments.

Some of the analyses that are performed to assess the patient's dental health are oral health condition progression analyses that compare oral conditions of the patient at multiple different points in time. For example, one carries assessment analysis may include comparing caries at a first point in time and a second point in time to determine a change in severity of the caries between the two points in time, if any. Other time-based comparative analyses that may be performed include a time-based comparison of gum recession, a time-based comparison of tooth wear, a time-based comparison of tooth movement, a time-based comparison of tooth staining, and so on. In some embodiments, processing logic automatically selects data collected at different points in time to perform such time-based analyses. Alternatively, a user may manually select data from one or more points in time to use for performing such time-based analyses.

In one embodiment, the different types of oral conditions for which analyses are performed and that are included in the detected oral conditions include tooth cracks, gum recession, tooth wear, occlusal contacts, crowding and/or spacing of teeth and/or other malocclusions, plaque, tooth stains, calculus, bone loss, bridges, fillings, implants, crowns, impacted teeth, root-canal fillings, gingival recession, TMD, and caries. Additional, fewer and/or alternative oral conditions may also be analyzed and reported. In embodiments, multiple different types of analyses are performed to determine presence, location and/or severity of one or more of the oral conditions. One type of analysis that may be performed is a point-in-time analysis that identifies the presence and/or severity levels of one or more oral conditions at a particular point-in-time based on data generated at that point-in-time (e.g., at block 1862). For example, a single x-ray image, CBCT scan, CT scan, MRI scan, intraoral scan, 3D model, intraoral image, etc. of a patient may be analyzed to determine whether, at a particular point-in-time, a patient's dental arch included any caries, gum recession, tooth wear, problem occlusion contacts, crowding, spacing or tooth gaps, plaque, tooth stains, TMD, gingival recession, and/or tooth cracks. Another type of analysis that may be performed is a time-based analysis that compares oral conditions at two or more points in time to determine changes in the oral conditions, progression of the oral conditions and/or rates of change of the oral conditions (e.g., at block 1864). For example, in embodiments a comparative analysis is performed to determine differences between x-rays, CBCT scans, CT scans, MRI scans, intraoral scans, 3D models, intraoral images, etc. taken at different points in time. The differences may be measured to determine an amount of change, and the amount of change together with the times at which the intraoral scans were taken may be used to determine a rate of change. This technique may be used, for example, to identify an amount of change and/or a rate of change for tooth wear, staining, plaque, crowding, spacing, gum recession, caries development, tooth cracks, and so on.

In embodiments, one or more trained models are used to perform at least some of the one or more oral condition analyses. The trained models may include physics models and/or AI models (e.g., machine learning models), for example. In one embodiment, a single model may be used to perform multiple different analyses (e.g., to identify any combination of tooth cracks, gum recession, tooth wear, occlusal contacts, crowding, TMD, gingival recession, and/or spacing of teeth and/or other malocclusions, plaque, tooth stains, and/or caries). Additionally, or alternatively, different models may be used to identify different oral conditions. For example, a first model may be used to identify tooth cracks, a second model may be used to identify tooth wear, a third model may be used to identify gum recession, a fourth model may be used to identify problem occlusal contacts, a fifth model may be used to identify crowding and/or spacing of teeth and/or other malocclusions, a sixth model may be used to identify plaque, a seventh model may be used to identify tooth stains, an eighth model may be used to identify TMD, a nineth model may be used to identify gingival recession, and/or a seventh model may be used to identify caries. Additionally, one or more rules based engines or applications (e.g., that are not based on machine learning) may be used in addition to, or instead of, one or more ML models for the identification and/or assessment of one or more oral conditions.

In one embodiment, at block 1862 intraoral data from one or more points in time are input into one or more trained machine learning models that have been trained to receive the intraoral data as an input and to output classifications of one or more types of oral conditions. In one embodiment, the trained machine learning model(s) is trained to identify areas of interest (AOIs) from the input intraoral data and to classify the AOIs based on oral conditions. The AOIs may be or include regions associated with particular oral conditions. The regions may include nearby or adjacent pixels or points that satisfy some criteria, for example. The intraoral data that is input into the one or more trained machine learning model may include three-dimensional (3D) data and/or two-dimensional (2D) data. The intraoral data may include, for example, one or more 3D models of a dental arch, one or more projections of one or more 3D models of a dental arch onto one or more planes (optionally comprising height maps), one or more x-rays of teeth, one or more CBCT scans, a panoramic x-ray, near-infrared and/or infrared imaging data, color image(s), ultraviolet imaging data, intraoral scans, one or more bitewing x-rays, one or more periapical x-rays, and so on. In some embodiments, a temporal sequence of 2D images generated from 3D data is input into the one or more AI models. If data from multiple imaging modalities are used (e.g., panoramic x-rays, bitewing x-rays, periapical x-rays, CBCT scans, 3D scan data, color images, and NIRI imaging data), then the data may be registered and/or stitched together so that the data is in a common reference frame and objects in the data are correctly positioned and oriented relative to objects in other data.

The trained AI model(s) may output segmentation information in embodiments. Segmentation information may be output for individual 2D images in a temporal sequence of 2D images generated from 3D data, and/or may be output for the 3D data from which the temporal sequence of 2D images was generated. In some embodiments, one or more AI models output a probability map, where each point in the probability map corresponds to a point in the intraoral data (e.g., a pixel in an intraoral image or point on a 3D surface) and indicates probabilities that the point represents one or more dental classes. In one embodiment, a single model outputs probabilities associated with multiple different types of dental classes, which includes one or more oral health condition classes. In an example, a trained machine learning model may output a probability map with probability values for a teeth dental class and a gums dental class. The probability map may further include probability values for tooth cracks, gum recession, tooth wear, occlusal contacts, crowding and/or spacing of teeth and/or other malocclusions, plaque, tooth stains, healthy area (e.g., healthy tooth and/or healthy gum), TMD, and/or caries. In the case of a single machine learning model that can identify each of tooth cracks, gum recession, tooth wear, occlusal contacts, crowding, TMD, and/or spacing of teeth and/or other malocclusions, plaque, tooth stains, and caries, eleven valued labels may be generated for each pixel, one for each of teeth, gums, healthy area, tooth cracks, gum recession, tooth wear, occlusal contacts, crowding and/or spacing of teeth and/or other malocclusions, plaque, tooth stains, and caries. In some embodiments, the corresponding predictions have a probability nature: for each pixel there are multiple numbers that may sum up to 1.0 and can be interpreted as probabilities of the pixel to correspond to these classes. In one embodiment, the first two values for teeth and gums sum up to 1.0 and the remaining values for healthy area, tooth cracks, gum recession, tooth wear, occlusal contacts, crowding and/or spacing of teeth and/or other malocclusions, plaque, tooth stains, and/or caries sum up to 1.0.

In some instances, multiple machine learning models are used, where each machine learning model identifies a subset of the possible oral conditions. For example, a first trained machine learning model may be trained to output a probability map with three values, one each for healthy teeth, gums, and caries. Alternatively, the first trained machine learning model may be trained to output a probability map with two values, one each for healthy teeth and caries. A second trained machine learning model may be trained to output a probability map with three values (one each for healthy teeth, gums and tooth cracks) or two values (one each for healthy teeth and tooth cracks). One or more additional trained machine learning models may each be trained to output probability maps associated with identifying specific types of oral conditions.

In embodiments, image processing and/or 3D data processing may be performed on radiographs, CBCT scan data, CT scan data, MRI scan data, intraoral scan data, 3D models, and/or other dental data. Such image processing and/or 3D data processing may be performed using one or more algorithms, which may be generic to multiple types of oral conditions or may be specific to particular oral conditions. For example, a trained model may identify regions on a dental radiograph or CBCT scan that include caries, and image processing may be performed to assess the size and/or severity of the identified caries. The image processing may include performing automated measurements such as size measurements, distance measurements, amount of change measurements, rate of change measurements, ratios, percentages, and so on. Accordingly, the image processing and/or 3D data processing may be performed to determine severity levels of oral conditions identified by the trained model(s). Alternatively, the trained models may be trained both to classify regions as caries and to identify a severity and/or size of the caries.

The one or more trained machine learning models that are used to identify, classify and/or determine a severity level for oral conditions may be neural networks such as deep neural networks or convolutional neural networks. Such machine learning models may be trained using supervised training in embodiments.

A dentist, after a quick glance at the dental diagnostics summary, may determine that a patient has carries, clinically significant tooth wear, and crowding/spacing and/or other malocclusions and/or oral conditions.

In embodiments, the oral health diagnostics system, and in particular the dental diagnostics summary, helps a doctor to quickly detect oral conditions (e.g., oral health conditions) and/or oral health problems and their respective severity levels, helps the doctor to make better judgments about treatment of oral conditions and/or oral health problems, and further helps the doctor in communicating with a patient that patient's oral conditions and/or oral health problems and possible treatments. This makes the process of identifying, diagnosing, and treating oral conditions and/or oral health problems easier and more efficient. The doctor may select any of the oral conditions and/or oral health problems to determine prognosis of that condition as it exists in the present and how it will likely progress into the future. Additionally, the oral health diagnostics system may provide treatment simulations of how the oral conditions and/or oral health problems will be affected or eliminated by one or more treatments.

In embodiments, a doctor may customize the oral conditions, oral health problems and/or areas of interest by adding emphasis or notes to specific oral conditions, oral health problems and/or areas of interest. For example, a patient may complain of a particular tooth aching. The doctor may highlight that particular tooth on a radiograph. Oral conditions that are found that are associated with the particular highlighted or selected tooth may then be shown in the dental diagnostics summary. In a further example, a doctor may select a particular tooth (e.g., lower left molar), and the dental diagnostics summary may be updated by modifying the severity results to be specific for that selected tooth. For example, if for the selected tooth an issue was found for caries and a possible issue was found for tooth stains, then the dental diagnostics summary would be updated to show no issues found for tooth wear, occlusion, crowding/spacing, plaque, tooth cracks, and gingival recession, to show a potential issue found for tooth stains and to show an issue found for caries. This may help a doctor to quickly identify possible root causes for the pain that the patient complained of for the specific tooth that was selected. The doctor may then select a different tooth to get a summary of dental issues for that other tooth.

Any of the methods (including user interfaces) described herein may be implemented as software, hardware or firmware, and may be described as a non-transitory machine-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, computer models (e.g., for additive manufacturing) and instructions related to forming a dental device may be stored on a non-transitory machine-readable storage medium.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but may be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware, or code set forth above may be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the foregoing specification, a detailed description has been given with reference to specific exemplary embodiments. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but may refer to different and distinct embodiments, as well as potentially the same embodiment.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media, and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that may include one or more digital processing devices and memory to store executable instructions to perform the operations described in this specification.

While this specification contains many specific embodiment details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A system comprising:
a memory device; and
a processing device to execute instructions from the memory device to:
receive data representing a potential for temporomandibular disorder (TMD) of a patient;
process the data to identify an indicator of the TMD;
identify a treatment recommendation based on the indicator of the TMD; and
provide the treatment recommendation for display on a user device.

2. The system of claim 1, wherein the data comprises at least one of audio data representing a sound of the potential for TMD of the patient, video data representing a video recording of the patient, or a cone-beam computed tomography (CBCT) scan of the patient.

3. The system of claim 2, wherein the video recording is captured as the patient performs at least one of opening, closing, lateral, or protrusive jaw movements.

4. The system of claim 2, wherein the audio data is captured while the patient performs at least one of opening, closing, lateral, or protrusive jaw movements.

5. The system of claim 2, wherein the CBCT scan is of a jaw of the patient, and wherein the CBCT scan represents the jaw of the patient in one of an open-jaw position or a closed-jaw position.

6. The system of claim 1, wherein the treatment recommendation comprises an aligner treatment that accommodates the TMD, wherein the aligner treatment comprises an aligner that is designed to reduce one or more symptoms of the TMD.

7. The system of claim 1, wherein the treatment recommendation comprises at one of: not implementing an aligner treatment, stopping an aligner treatment, or slowing down an aligner treatment.

8. The system of claim 1, wherein the processing device is further to:
providing instructions to fabricate an appliance based on the indicator of the TMD.

9. The system of claim 8, wherein the appliance comprises a 3D-printed appliance to correct the TMD.

10. The system of claim 9, wherein the appliance comprises a 3D-printed appliance to concurrently treat the TMD and orthodontically move teeth.

11. The system of claim 1, wherein the treatment recommendation comprises an appliance to correct the TMD.

12. The system of claim 1, wherein to process the data to identify the indicator of the TMD, the processing device is further to:
provide the data as input to a machine learning model that is trained to output a value representing a likelihood of the TMD.

13. The system of claim 1, wherein the user device corresponds to a patient device, a doctor device, or a scanning device.

14. The system of claim 1, wherein the system is comprised in at least one of: a scanning device, a patient device, or a doctor device.

15. The system of claim 1, wherein to identify the treatment recommendation based on the indicator of the TMD, the processing device is further to:
receive one or more responses to a patient questionnaire;
analyze the one or more responses to identify an additional indicator of the TMD; and
determine that the patient has the TMD based on a combination of the indicator of the TMD and the additional indicator of the TMD.

16. The system of claim 1, wherein the data comprises audio data representing a sound of the potential for TMD of the patient, and wherein the processing device is further to:
perform a preprocessing of the audio data, wherein the preprocessing comprises at least one of:
filtering the audio data to remove background noise;
extracting frequency data from the audio data, wherein the frequency data comprises a first frequency range corresponding to the sound of the TMD and a second frequency range not corresponding to the sound of the TMD;
amplifying the first frequency range;
reducing the second frequency range; or
converting the audio data to a spectrogram representing the frequency data over time.

17. The system of claim 1, wherein the data comprises video data, and wherein the processing device is further to:
segment each frame of the video data into a plurality of features;
identify, in each frame, a first feature of a head of the patient and a second feature of the head of the patient;
measure, for each frame, a distance between the first feature of the head and the second feature of the head;
determine a difference between a first distance for a first frame to a second distance for a second frame, wherein the first frame and the second frame are consecutive frames; and
responsive to determining that the difference satisfies a criterion, set the indicator to indicate presence of the TMD.

18. The system of claim 1, wherein the data comprises CBCT scan of a jaw of the patient, and wherein the processing device is further to:
segment the CBCT scan to identify a first region of the jaw of the patient and a second region of the jaw of the patient;
identify a first bone density represented in the first region and a second bone density represented in the second region;
determine a difference between the first bone density and the second bone density; and
responsive to determining that the difference satisfies a criterion, identify a presence of the TMD in the patient.

19. A method comprising:
receiving data representing a potential for temporomandibular disorder (TMD) of a patient;
processing the data to identify an indicator of the TMD;
identifying a treatment recommendation based on the indicator of the TMD; and
providing the treatment recommendation for display on a user device.

20. A non-transitory computer-readable storage medium comprising instructions that, when executed by a processing device, cause the processing device to perform operations comprising:
receiving data representing a potential for temporomandibular disorder (TMD) of a patient;
processing the data to identify an indicator of the TMD;
identifying a treatment recommendation based on the indicator of the TMD; and
providing the treatment recommendation for display on a user device.
